(19) United States
(12) Patent Application Publication
(10) Pub. No.: US 2026/0092073 A1
(43) Pub. Date: Apr. 2, 2026

(54) NOVEL COMPOUNDS

(71) Applicant: Hoffmann-La Roche Inc., Little Falls, NJ (US)

(72) Inventors: David BON, Nottingham (GB); Lea Aurelie BOUCHE, Basel (CH); Wolfgang GUBA, Müllheim (DE); Georg JAESCHKE, Basel (CH); Stefanie Katharina MESCH, Basel (CH); Angélique PATINY-ADAM, Kembs (FR); Christian SCHNIDER, Biel-Benken (CH); Jonathan Martin SHANNON, Nottingham (GB); Sandra STEINER, Sursee (CH); Andreas Michael TOSSTORFF, Muenchenstein (CH)

(73) Assignee: Hoffmann-La Roche Inc., Little Falls, NJ (US)

(21) Appl. No.: 19/358,658

(22) Filed: Oct. 15, 2025

Related U.S. Application Data
(63) Continuation of application No. PCT/EP2024/060319, filed on Apr. 16, 2024.

(30) Foreign Application Priority Data
Apr. 19, 2023 (EP) 23168774.0
Nov. 1, 2023 (EP) 23207285.0

(51) Int. Cl. C07D 498/04

(52) U.S. Cl. CPC C07D 498/04

(57) ABSTRACT

The invention relates to novel compounds having the general formula I

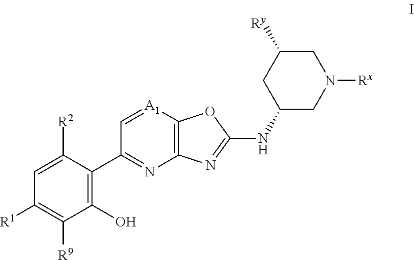

wherein R¹, R², R⁹, A₁, R^{x} and R^{y} are as described herein, composition including the compounds and methods of using the compounds.

### FIELD OF THE INVENTION

The present invention relates to organic compounds useful for therapy and/or prophylaxis in a mammal, and in particular to compounds that modulate NLRP3 inhibition.

The present invention provides novel compounds of formula I

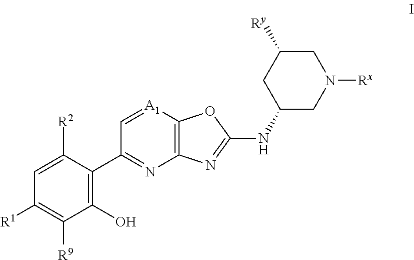

wherein,
R¹ is cyano, halo, or haloalkyl, and R⁹ is H, or
R¹ and R⁹ and the atoms to which they are attached form a 5-membered cycloalkyl or a 5-membered heterocycle comprising 1 O heteroatom;
R² is alkyl, hydroxyalkyl, alkoxyalkyl or cyano, wherein if R¹ is cyano then R² is not cyano;
A₁ is —CR¹⁰— or —N—;
R¹⁰ is H, fluoro or alkoxy;
R^{x} is H, alkyl, heteroarylalkyl, hydroxyalkyl or COOH-alkyl, wherein the heteroaryl of the heteroarylalkyl is oxazolyl, tetrazolyl or oxadiazolyl;
R^{y} is H or —OH;
   wherein, if R¹ is haloalkyl, then R^{x} is hydroxyalkyl or COOH-alkyl and R^{y} is —OH;
and pharmaceutically acceptable salts thereof, provided that the compound is not a compound selected from:
5-Chloro-2-[2-[[(3R)-1-ethyl-3-piperidyl]amino]oxazolo[4,5-b]pyridin-5-yl]-3-methyl-phenol;
5-Chloro-2-[2-[[(3R)-1-ethyl-3-piperidyl]amino]oxazolo[4,5-b]pyridin-5-yl]-3-hydroxy-benzonitrile;
5-Chloro-2-[2-[[(3R)-1-ethyl-3-piperidyl]amino]oxazolo[4,5-b]pyridin-5-yl]-3-(methoxymethyl)phenol;2,2,2-trifluoroacetic acid;
5-Chloro-2-[2-[[(3R)-1-ethyl-3-piperidyl]amino]oxazolo[4,5-b]pyridin-5-yl]-3-(methoxymethyl)phenol;
5-Chloro-3-methyl-2-[2-[[(3R)-1-methyl-3-piperidyl]amino]oxazolo[4,5-b]pyridin-5-yl]phenol;
5-Chloro-3-methyl-2-[2-[[(3R)-1-propyl-3-piperidyl]amino]oxazolo[4,5-b]pyridin-5-yl]phenol;
(3S,5R)-5-[[5-(4-Chloro-2-hydroxy-6-methyl-phenyl)oxazolo[4,5-b]pyridin-2-yl]amino]-1-methyl-piperidin-3-ol;
5-Chloro-2-[2-[[(3R,5S)-1-ethyl-5-fluoro-3-piperidyl]amino]oxazolo[4,5-b]pyridin-5-yl]-3-methyl-phenol;
(3R,5R)-5-[[5-(4-Chloro-2-hydroxy-6-methyl-phenyl)oxazolo[4,5-b]pyridin-2-yl]amino]-1-ethyl-piperidin-3-ol;formic acid;
(3R,5R)-5-[[5-(4-Chloro-2-hydroxy-6-methyl-phenyl)oxazolo[4,5-b]pyridin-2-yl]amino]-1-ethyl-piperidin-3-ol;
5-Chloro-2-[2-[[(3R)-1-(3,3-difluoropropyl)-3-piperidyl]amino]oxazolo[4,5-b]pyridin-5-yl]-3-methyl-phenol;
5-Chloro-2-[2-[[(3R)-1-(2-hydroxyethyl)-3-piperidyl]amino]oxazolo[4,5-b]pyridin-5-yl]-3-methyl-phenol;
5-Chloro-3-(hydroxymethyl)-2-[2-[[(3R)-1-methyl-3-piperidyl]amino]oxazolo[4,5-b]pyridin-5-yl]phenol;
5-Chloro-2-[2-[[(3R)-1-(3-hydroxypropyl)-3-piperidyl]amino]oxazolo[4,5-b]pyridin-5-yl]-3-methyl-phenol;
5-Chloro-3-methyl-2-[2-[[(3R)-1-(2-hydroxypropyl)-3-piperidyl]amino]oxazolo[4,5-b]pyridin-5-yl]phenol;
(3S,5R)-5-[[5-[4-Chloro-2-hydroxy-6-(methoxymethyl)phenyl]oxazolo[4,5-b]pyridin-2-yl]amino]-1-methyl-piperidin-3-ol;
5-Chloro-2-[2-[[(3R)-1-ethyl-3-piperidyl]amino]oxazolo[4,5-b]pyrazin-5-yl]-3-methyl-phenol;2,2,2-trifluoroacetic acid;
5-Chloro-2-[2-[[(3R)-1-[[trans-2-hydroxycyclobutyl]methyl]-3-piperidyl]amino]oxazolo[4,5-b]pyridin-5-yl]-3-methyl-phenol or 5-chloro-2-[2-[[(3R)-1-[[cis-2-hydroxycyclobutyl]methyl]-3-piperidyl]amino]oxazolo[4,5-b]pyridin-5-yl]-3-methyl-phenol;
(3S,5R)-5-[[5-(4-Chloro-2-hydroxy-6-methyl-phenyl)oxazolo[4,5-b]pyridin-2-yl]amino]-1-ethyl-piperidin-3-ol;
2-[2-[[(3R)-1-Ethyl-3-piperidyl]amino]oxazolo[4,5-b]pyridin-5-yl]-5-fluoro-3-methyl-phenol;
5-Fluoro-3-methyl-2-[2-[[(3R)-1-methyl-3-piperidyl]amino]oxazolo[4,5-b]pyridin-5-yl]phenol;
4-[2-[[(3R)-1-Ethyl-3-piperidyl]amino]oxazolo[4,5-b]pyridin-5-yl]-3-hydroxy-5-methyl-benzonitrile;
3-Hydroxy-5-methyl-4-[2-[[(3R)-1-methyl-3-piperidyl]amino]oxazolo[4,5-b]pyridin-5-yl]benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;formic acid;
4-[2-[[(3R,5S)-1-Ethyl-5-hydroxy-3-piperidyl]amino]oxazolo[4,5-b]pyridin-5-yl]-3-hydroxy-5-methyl-benzonitrile;
3-Hydroxy-4-[2-[[(3R,5S)-5-hydroxy-1-methyl-3-piperidyl]amino]oxazolo[4,5-b]pyridin-5-yl]-5-methyl-benzonitrile;
4-[2-[[(3R,5R)-1-Ethyl-5-hydroxy-3-piperidyl]amino]oxazolo[4,5-b]pyridin-5-yl]-3-hydroxy-5-methyl-benzonitrile;
3-Hydroxy-4-[2-[[(3R)-1-(2-hydroxyethyl)-3-piperidyl]amino]oxazolo[4,5-b]pyridin-5-yl]-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid
4-[2-[[(3R)-1-Ethyl-3-piperidyl]amino]oxazolo[4,5-b]pyridin-5-yl]-3-hydroxy-5-(methoxymethyl)benzonitrile;
4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid;
4-[2-[[(3R)-1-Ethyl-3-piperidyl]amino]-7-methoxy-oxazolo[4,5-b]pyridin-5-yl]-3-hydroxy-5-methyl-benzonitrile;
5-[2-[[(3R)-1-Ethyl-3-piperidyl]amino]oxazolo[4,5-b]pyridin-5-yl]-6-methyl-2,3-dihydrobenzofuran-4-ol;
5-Chloro-3-methyl-2-[2-[[(3R)-3-piperidyl]amino]oxazolo[4,5-b]pyridin-5-yl]phenol;dihydrochloride;
5-Chloro-3-methyl-2-[2-[[(3R)-3-piperidyl]amino]oxazolo[4,5-b]pyridin-5-yl]phenol;
and pharmaceutically acceptable salts thereof.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1a. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

WO2023/066825 discloses compounds useful as NLRP3 inhibitors. However, there is a need to provide compounds with improved pharmacological and/or physiological and/or physicochemical properties and/or those that provide a useful alternative to known compounds. Further, in developing a NLRP3 inhibitor for treating peripheral indications, it is advantageous to minimize the exposure of a NLRP3 inhibiting compound in the brain relative to systemic exposure to minimize the risk of potential side effects in the central nervous system (CNS). P-gp (P-glycoprotein) is an important transporter that is expressed in the capillary endothelial cells composing the blood-brain barrier and blood-testis barrier, where it pumps xenobiotics back into the capillaries and limiting brain exposure. The compounds of formula I achieve peripheral preference by showing an increased efflux in a transcellular assay expressing active P-gp transporters and/or reduced passive permeability without compromising systemic distribution. By slightly increasing the polar surface area the compounds of formula I surprising yield the desired profile and demonstrate lower permeability. In addition there was a need for compounds showing a reduced phospholipidosis risk for chronic indications with compounds that display a combination of increased polarity and fine-tuned pKa.

### SUMMARY OF THE INVENTION

The present invention provides novel compounds of formula I

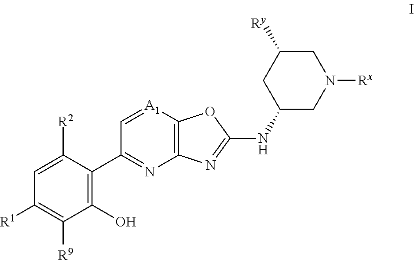

wherein,
R¹ is cyano, halo, or haloalkyl, and R⁹ is H, or
R¹ and R⁹ and the atoms to which they are attached form a 5-membered cycloalkyl or a 5-membered heterocycle comprising 1 O heteroatom;
R² is alkyl, hydroxyalkyl, alkoxyalkyl or cyano, wherein if R¹ is cyano then R² is not cyano;
A₁ is —CR¹⁰— or —N—;
R¹⁰ is H, fluoro or alkoxy;
R^{x} is H, alkyl, heteroarylalkyl, hydroxyalkyl or COOH-alkyl, wherein the heteroaryl of the heteroarylalkyl is oxazolyl, tetrazolyl or oxadiazolyl;
R^{y} is H or —OH;
   wherein, if R¹ is haloalkyl, then R^{x} is hydroxyalkyl or COOH-alkyl and R^{y} is —OH;
and pharmaceutically acceptable salts thereof, provided that the compound is not a compound selected from:
5-Chloro-2-[2-[[(3R)-1-ethyl-3-piperidyl]amino]oxazolo[4,5-b]pyridin-5-yl]-3-methyl-phenol;
5-Chloro-2-[2-[[(3R)-1-ethyl-3-piperidyl]amino]oxazolo[4,5-b]pyridin-5-yl]-3-hydroxy-benzonitrile;
5-Chloro-2-[2-[[(3R)-1-ethyl-3-piperidyl]amino]oxazolo[4,5-b]pyridin-5-yl]-3-(methoxymethyl)phenol;2,2,2-trifluoroacetic acid;
5-Chloro-2-[2-[[(3R)-1-ethyl-3-piperidyl]amino]oxazolo[4,5-b]pyridin-5-yl]-3-(methoxymethyl)phenol;
5-Chloro-3-methyl-2-[2-[[(3R)-1-methyl-3-piperidyl]amino]oxazolo[4,5-b]pyridin-5-yl]phenol;
5-Chloro-3-methyl-2-[2-[[(3R)-1-propyl-3-piperidyl]amino]oxazolo[4,5-b]pyridin-5-yl]phenol;
(3S,5R)-5-[[5-(4-Chloro-2-hydroxy-6-methyl-phenyl)oxazolo[4,5-b]pyridin-2-yl]amino]-1-methyl-piperidin-3-ol;
5-Chloro-2-[2-[[(3R,5S)-1-ethyl-5-fluoro-3-piperidyl]amino]oxazolo[4,5-b]pyridin-5-yl]-3-methyl-phenol;
(3R,5R)-5-[[5-(4-Chloro-2-hydroxy-6-methyl-phenyl)oxazolo[4,5-b]pyridin-2-yl]amino]-1-ethyl-piperidin-3-ol;formic acid;
(3R,5R)-5-[[5-(4-Chloro-2-hydroxy-6-methyl-phenyl)oxazolo[4,5-b]pyridin-2-yl]amino]-1-ethyl-piperidin-3-ol;
5-Chloro-2-[2-[[(3R)-1-(3,3-difluoropropyl)-3-piperidyl]amino]oxazolo[4,5-b]pyridin-5-yl]-3-methyl-phenol;
5-Chloro-2-[2-[[(3R)-1-(2-hydroxyethyl)-3-piperidyl]amino]oxazolo[4,5-b]pyridin-5-yl]-3-methyl-phenol;
5-Chloro-3-(hydroxymethyl)-2-[2-[[(3R)-1-methyl-3-piperidyl]amino]oxazolo[4,5-b]pyridin-5-yl]phenol;
5-Chloro-2-[2-[[(3R)-1-(3-hydroxypropyl)-3-piperidyl]amino]oxazolo[4,5-b]pyridin-5-yl]-3-methyl-phenol;
5-Chloro-3-methyl-2-[2-[[(3R)-1-(2-hydroxypropyl)-3-piperidyl]amino]oxazolo[4,5-b]pyridin-5-yl]phenol;
(3S,5R)-5-[[5-[4-Chloro-2-hydroxy-6-(methoxymethyl)phenyl]oxazolo[4,5-b]pyridin-2-yl]amino]-1-methyl-piperidin-3-ol;
5-Chloro-2-[2-[[(3R)-1-ethyl-3-piperidyl]amino]oxazolo[4,5-b]pyrazin-5-yl]-3-methyl-phenol;2,2,2-trifluoroacetic acid;
5-Chloro-2-[2-[[(3R)-1-[[trans-2-hydroxycyclobutyl]methyl]-3-piperidyl]amino]oxazolo[4,5-b]pyridin-5-yl]-3-methyl-phenol or 5-chloro-2-[2-[[(3R)-1-[[cis-2-hydroxycyclobutyl]methyl]-3-piperidyl]amino]oxazolo[4,5-b]pyridin-5-yl]-3-methyl-phenol;
(3S,5R)-5-[[5-(4-Chloro-2-hydroxy-6-methyl-phenyl)oxazolo[4,5-b]pyridin-2-yl]amino]-1-ethyl-piperidin-3-ol;
2-[2-[[(3R)-1-Ethyl-3-piperidyl]amino]oxazolo[4,5-b]pyridin-5-yl]-5-fluoro-3-methyl-phenol;
5-Fluoro-3-methyl-2-[2-[[(3R)-1-methyl-3-piperidyl]amino]oxazolo[4,5-b]pyridin-5-yl]phenol;
4-[2-[[(3R)-1-Ethyl-3-piperidyl]amino]oxazolo[4,5-b]pyridin-5-yl]-3-hydroxy-5-methyl-benzonitrile;
3-Hydroxy-5-methyl-4-[2-[[(3R)-1-methyl-3-piperidyl]amino]oxazolo[4,5-b]pyridin-5-yl]benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;formic acid;
4-[2-[[(3R,5S)-1-Ethyl-5-hydroxy-3-piperidyl]amino]oxazolo[4,5-b]pyridin-5-yl]-3-hydroxy-5-methyl-benzonitrile;
3-Hydroxy-4-[2-[[(3R,5S)-5-hydroxy-1-methyl-3-piperidyl]amino]oxazolo[4,5-b]pyridin-5-yl]-5-methyl-benzonitrile;
4-[2-[[(3R,5R)-1-Ethyl-5-hydroxy-3-piperidyl]amino]oxazolo[4,5-b]pyridin-5-yl]-3-hydroxy-5-methyl-benzonitrile;
3-Hydroxy-4-[2-[[(3R)-1-(2-hydroxyethyl)-3-piperidyl]amino]oxazolo[4,5-b]pyridin-5-yl]-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid
4-[2-[[(3R)-1-Ethyl-3-piperidyl]amino]oxazolo[4,5-b]pyridin-5-yl]-3-hydroxy-5-(methoxymethyl)benzonitrile;
4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid;
4-[2-[[(3R)-1-Ethyl-3-piperidyl]amino]-7-methoxy-oxazolo[4,5-b]pyridin-5-yl]-3-hydroxy-5-methyl-benzonitrile;
5-[2-[[(3R)-1-Ethyl-3-piperidyl]amino]oxazolo[4,5-b]pyridin-5-yl]-6-methyl-2,3-dihydrobenzofuran-4-ol;
5-Chloro-3-methyl-2-[2-[[(3R)-3-piperidyl]amino]oxazolo[4,5-b]pyridin-5-yl]phenol;dihydrochloride;
5-Chloro-3-methyl-2-[2-[[(3R)-3-piperidyl]amino]oxazolo[4,5-b]pyridin-5-yl]phenol;
and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups are methyl and ethyl.

The term “alkoxy” denotes a group of the formula —O—R′, wherein R′ is a C₁-6-alkyl group. Examples of C₁₋₆-alkoxy groups include methoxy, ethoxy, n-propoxy, isopropoxy, n-butoxy, isobutoxy and tert-butoxy.

The term “alkoxyalkyl” denotes an alkyl group wherein one of the hydrogen atoms of the alkyl group have been replaced by an alkoxy group. Examples of alkoxyalkyl are methoxymethyl and methoxyethyl. Particular alkoxyalkyl is methoxymethyl.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. Examples of cycloalkyl groups include cyclopropyl, cyclobutyl, cyclopentyl and cyclohexyl, and the like.

The term “halogen”, “halide” and “halo” are used interchangeably herein and denote fluoro, chloro, bromo or iodo.

The term “haloalkyl” denotes an alkyl group wherein one or more of the hydrogen atoms of the alkyl group have been replaced by halo. Examples of haloalkyl are trifluoromethyl, difluromethyl and fluoromethyl. Particular example is trifluromethyl.

The term “heteroaryl” denotes a monovalent aromatic mono- or bicyclic ring system of 4 to 9 ring atoms, comprising 1, 2, 3, or 4 ring heteroatoms selected from N and O, the remaining ring atoms being carbon. Examples of heteroaryl are thiazolyl, pyridyl, oxazolyl, tetrazolyl, oxadiazolyl and the like.

The term “heteroarylalkyl”, denotes an alkyl group wherein one of the hydrogen atoms of the alkyl group has been replaced by a heteroaryl group. Examples are oxyzolylethyl, tetrazolylethyl, and oxadiazolylethyl. Particular example is oxazolylethyl.

The term “heterocycle ring” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Examples for monocyclic saturated heterocycle rings are azetidinyl, diazepanyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, morpholinyl, and piperazinyl. Examples of polycyclic saturated heterocycle rings are azaspiroheptanyl, diazaspiroheptanyl, azaspirooctanyl, diazospirooctanyl, diazaspirononanyl, oxaazaspirooctanyl, and oxadiazaspirononanyl.

The term “hydroxy” denotes a —OH group.

The term “hydroxyalkyl” denotes an alkyl group wherein at least one of the hydrogen atoms of the alkyl group has been replaced by a hydroxy group. Examples of hydroxyalkyl include hydroxymethyl, hydroxyethyl, hydroxypropyl, hydroxymethylethyl, hydroxymethylpropyl and dihydroxypropyl. Particular example of hydroxyalkyl is hydroxyethyl.

The term “cyano” denotes a —C≡N group.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol pg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I

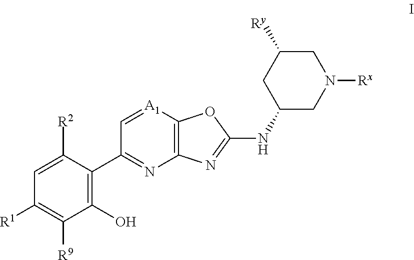

wherein,
R¹ is cyano, halo, or haloalkyl, and R⁹ is H, or
R¹ and R⁹ and the atoms to which they are attached form a 5-membered cycloalkyl or a 5-membered heterocycle comprising 1 O heteroatom;
R² is alkyl, hydroxyalkyl, alkoxyalkyl or cyano, wherein if R¹ is cyano then R² is not cyano;
A₁ is —CR¹⁰— or —N—;
R¹⁰ is H, fluoro or alkoxy;
R^{x} is H, alkyl, heteroarylalkyl, hydroxyalkyl or COOH-alkyl, wherein the heteroaryl of the heteroarylalkyl is oxazolyl, tetrazolyl or oxadiazolyl;
R^{y} is H or —OH;
wherein, if R¹ is haloalkyl, then R^{x} is hydroxyalkyl or COOH-alkyl and R^{y} is —OH;
and pharmaceutically acceptable salts thereof, provided that the compound is not a compound selected from:
5-Chloro-2-[2-[[(3R)-1-ethyl-3-piperidyl]amino]oxazolo[4,5-b]pyridin-5-yl]-3-methyl-phenol;
5-Chloro-2-[2-[[(3R)-1-ethyl-3-piperidyl]amino]oxazolo[4,5-b]pyridin-5-yl]-3-hydroxy-benzonitrile;
5-Chloro-2-[2-[[(3R)-1-ethyl-3-piperidyl]amino]oxazolo[4,5-b]pyridin-5-yl]-3-(methoxymethyl)phenol;2,2,2-trifluoroacetic acid;
5-Chloro-2-[2-[[(3R)-1-ethyl-3-piperidyl]amino]oxazolo[4,5-b]pyridin-5-yl]-3-(methoxymethyl)phenol;
5-Chloro-3-methyl-2-[2-[[(3R)-1-methyl-3-piperidyl]amino]oxazolo[4,5-b]pyridin-5-yl]phenol;
5-Chloro-3-methyl-2-[2-[[(3R)-1-propyl-3-piperidyl]amino]oxazolo[4,5-b]pyridin-5-yl]phenol;
(3S,5R)-5-[[5-(4-Chloro-2-hydroxy-6-methyl-phenyl)oxazolo[4,5-b]pyridin-2-yl]amino]-1-methyl-piperidin-3-ol;
(3R,5R)-5-[[5-(4-Chloro-2-hydroxy-6-methyl-phenyl)oxazolo[4,5-b]pyridin-2-yl]amino]-1-ethyl-piperidin-3-ol;formic acid;
(3R,5R)-5-[[5-(4-Chloro-2-hydroxy-6-methyl-phenyl)oxazolo[4,5-b]pyridin-2-yl]amino]-1-ethyl-piperidin-3-ol;
5-Chloro-2-[2-[[(3R)-1-(3,3-difluoropropyl)-3-piperidyl]amino]oxazolo[4,5-b]pyridin-5-yl]-3-methyl-phenol;
5-Chloro-2-[2-[[(3R)-1-(2-hydroxyethyl)-3-piperidyl]amino]oxazolo[4,5-b]pyridin-5-yl]-3-methyl-phenol;
5-Chloro-3-(hydroxymethyl)-2-[2-[[(3R)-1-methyl-3-piperidyl]amino]oxazolo[4,5-b]pyridin-5-yl]phenol;
5-Chloro-2-[2-[[(3R)-1-(3-hydroxypropyl)-3-piperidyl]amino]oxazolo[4,5-b]pyridin-5-yl]-3-methyl-phenol;
5-Chloro-3-methyl-2-[2-[[(3R)-1-(2-hydroxypropyl)-3-piperidyl]amino]oxazolo[4,5-b]pyridin-5-yl]phenol;
(3S,5R)-5-[[5-[4-Chloro-2-hydroxy-6-(methoxymethyl)phenyl]oxazolo[4,5-b]pyridin-2-yl]amino]-1-methyl-piperidin-3-ol;
5-Chloro-2-[2-[[(3R)-1-ethyl-3-piperidyl]amino]oxazolo[4,5-b]pyrazin-5-yl]-3-methyl-phenol;2,2,2-trifluoroacetic acid;
5-Chloro-2-[2-[[(3R)-1-ethyl-3-piperidyl]amino]oxazolo[4,5-b]pyrazin-5-yl]-3-methyl-phenol;
(3S,5R)-5-[[5-(4-Chloro-2-hydroxy-6-methyl-phenyl)oxazolo[4,5-b]pyridin-2-yl]amino]-1-ethyl-piperidin-3-ol;
2-[2-[[(3R)-1-Ethyl-3-piperidyl]amino]oxazolo[4,5-b]pyridin-5-yl]-5-fluoro-3-methyl-phenol;
5-Fluoro-3-methyl-2-[2-[[(3R)-1-methyl-3-piperidyl]amino]oxazolo[4,5-b]pyridin-5-yl]phenol;
4-[2-[[(3R)-1-Ethyl-3-piperidyl]amino]oxazolo[4,5-b]pyridin-5-yl]-3-hydroxy-5-methyl-benzonitrile;
3-Hydroxy-5-methyl-4-[2-[[(3R)-1-methyl-3-piperidyl]amino]oxazolo[4,5-b]pyridin-5-yl]benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;formic acid;
4-[2-[[(3R,5S)-1-Ethyl-5-hydroxy-3-piperidyl]amino]oxazolo[4,5-b]pyridin-5-yl]-3-hydroxy-5-methyl-benzonitrile;
3-Hydroxy-4-[2-[[(3R,5S)-5-hydroxy-1-methyl-3-piperidyl]amino]oxazolo[4,5-b]pyridin-5-yl]-5-methyl-benzonitrile;
4-[2-[[(3R,5R)-1-Ethyl-5-hydroxy-3-piperidyl]amino]oxazolo[4,5-b]pyridin-5-yl]-3-hydroxy-5-methyl-benzonitrile;
3-Hydroxy-4-[2-[[(3R)-1-(2-hydroxyethyl)-3-piperidyl]amino]oxazolo[4,5-b]pyridin-5-yl]-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid
4-[2-[[(3R)-1-Ethyl-3-piperidyl]amino]oxazolo[4,5-b]pyridin-5-yl]-3-hydroxy-5-(methoxymethyl)benzonitrile;
4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid;
4-[2-[[(3R)-1-Ethyl-3-piperidyl]amino]-7-methoxy-oxazolo[4,5-b]pyridin-5-yl]-3-hydroxy-5-methyl-benzonitrile;
5-[2-[[(3R)-1-Ethyl-3-piperidyl]amino]oxazolo[4,5-b]pyridin-5-yl]-6-methyl-2,3-dihydrobenzofuran-4-ol;
5-Chloro-3-methyl-2-[2-[[(3R)-3-piperidyl]amino]oxazolo[4,5-b]pyridin-5-yl]phenol;dihydrochloride;
5-Chloro-3-methyl-2-[2-[[(3R)-3-piperidyl]amino]oxazolo[4,5-b]pyridin-5-yl]phenol;
and pharmaceutically acceptable salts thereof.

An embodiment of the present invention provides compounds according to formula I as described herein, wherein R¹ is cyano or haloalkyl, and R⁹ is H, or R¹ and R⁹ and the atoms to which they are attached form a 5-membered heterocycle comprising 1 O heteroatom.

An embodiment of the present invention provides compounds according to formula I as described herein, wherein R¹ is cyano and R⁹ is H, or R¹ and R⁹ and the atoms to which they are attached form a 5-membered heterocycle comprising 1 O heteroatom.

An embodiment of the present invention provides compounds according to formula I as described herein, wherein R¹ is cyano and R⁹ is H.

An embodiment of the present invention provides compounds according to formula I as described herein, wherein R¹ and R⁹ and the atoms to which they are attached form a 5-membered heterocycle comprising 1 O heteroatom.

An embodiment of the present invention provides compounds according to formula I as described herein, wherein R² is alkyl or alkoxyalkyl.

An embodiment of the present invention provides compounds according to formula I as describe herein, wherein A₁ is —N—.

An embodiment of the present invention provides compounds according to formula I as described herein, wherein R¹⁰ is H or alkoxy.

An embodiment of the present invention provides compounds according to formula I as described herein, wherein R¹⁰ is H.

An embodiment of the present invention provides compounds according to formula I as described herein, wherein R^{x} is H, alkyl, heteroarylalkyl, hydroxyalkyl or COOH-alkyl, wherein the heteroaryl of heteroarylalkyl is oxazolyl.

An embodiment of the present invention provides compounds according to formula I as described herein, wherein R^{x} is H, alkyl, heteroarylalkyl or hydroxyalkyl, wherein the heteroaryl of heteroarylalkyl is oxazolyl.

An embodiment of the present invention provides compounds according to formula I as described herein, wherein R^{x} is alkyl, heteroarylalkyl or hydroxyalkyl, wherein the heteroaryl of heteroarylalkyl is oxazolyl.

An embodiment of the present invention provides compounds according to formula I as described herein, wherein R^{x} is alkyl or hydroxyalkyl.

An embodiment of the present invention provides compounds according to formula I as described herein, wherein R^{x} is hydroxyalkyl.

An embodiment of the present invention provides compounds according to formula I as described herein, wherein R^{y} is H.

An embodiment of the present invention provides compounds according to formula I as described herein, wherein,
R¹ is cyano or haloalkyl, and R⁹ is H, or
R¹ and R⁹ and the atoms to which they are attached form a 5-membered heterocycle comprising 1 O heteroatom;
R² is alkyl or alkoxyalkyl;
A₁ is —CR¹⁰— or —N—;
R¹⁰ is H or alkoxy;
R^{x} is H, alkyl, heteroarylalkyl, hydroxyalkyl or COOH-alkyl, wherein the heteroaryl of the heteroarylalkyl is oxazolyl;
R^{y} is H or —OH;
wherein, if R¹ is haloalkyl, then R^{x} is hydroxyalkyl or COOH-alkyl and R^{y} is —OH;
and pharmaceutically acceptable salts thereof.

An embodiment of the present invention provides compounds according to formula I as described herein, wherein,
R¹ is cyano and R⁹ is H, or
R¹ and R⁹ and the atoms to which they are attached form a 5-membered heterocycle comprising 1 O heteroatom;
R² is alkyl or alkoxyalkyl;
A₁ is —CR¹⁰— or —N—;
R¹⁰ is H or alkoxy;
R^{x} is alkyl or hydroxyalkyl;
R^{y} is H or —OH;
and pharmaceutically acceptable salts thereof.

An embodiment of the present invention provides compounds according to formula I as described herein, wherein,
R¹ is cyano and R⁹ is H, or
R¹ and R⁹ and the atoms to which they are attached form a 5-membered heterocycle comprising 1 O heteroatom;
R² is alkyl or alkoxyalkyl;
A₁ is —CR¹⁰— or —N—;
R¹⁰ is H;
R^{x} is hydroxyalkyl;
R^{y} is H;
and pharmaceutically acceptable salts thereof.

An embodiment of the present invention provides compounds according to formula I as described herein, wherein,
R¹ and R⁹ and the atoms to which they are attached form a 5-membered heterocycle comprising 1 O heteroatom;
R² is alkyl or alkoxyalkyl;
A₁ is —CR¹⁰— or —N—;
R¹⁰ is H;
R^{x} is hydroxyalkyl;
R^{y} is H;
and pharmaceutically acceptable salts thereof.

An embodiment of the present invention provides compounds according to formula I as described herein, wherein,
R¹ and R⁹ and the atoms to which they are attached form a 5-membered heterocycle comprising 1 O heteroatom;
R² is alkyl or alkoxyalkyl;
A₁ is —N—;
R¹⁰ is H;
R^{x} is hydroxyalkyl;
R^{y} is H;
and pharmaceutically acceptable salts thereof.

An embodiment of the present invention provides compounds according to formula I as described herein, wherein,
R¹ is cyano and R⁹ is H;
R² is alkyl or alkoxyalkyl;
A₁ is —CR¹⁰— or —N—;
R¹⁰ is H or alkoxy;
R^{x} is H, alkyl, heteroarylalkyl or hydroxyalkyl, wherein the heteroaryl of the heteroarylalkyl is oxazolyl;
R^{y} is H or —OH;
and pharmaceutically acceptable salts thereof.

An embodiment of the present invention provides compounds according to formula I as described herein, wherein,
R¹ is cyano and R⁹ is H;
R² is alkyl or alkoxyalkyl;
A₁ is —N—;
R¹⁰ is H or alkoxy;
R^{x} is H, alkyl, heteroarylalkyl or hydroxyalkyl, wherein the heteroaryl of the heteroarylalkyl is oxazolyl;
R^{y} is H or —OH;
and pharmaceutically acceptable salts thereof.

An embodiment of the present invention provides compounds according to formula I as described herein, wherein,
R¹ is cyano and R⁹ is H;
R² is alkyl or alkoxyalkyl;
A₁ is —CR¹⁰— or —N—;
R¹⁰ is H;
R^{x} is alkyl, heteroarylalkyl or hydroxyalkyl, wherein the heteroaryl of the heteroarylalkyl is oxazolyl;
R^{y} is H or —OH;
and pharmaceutically acceptable salts thereof.

An embodiment of the present invention provides compounds according to formula I as described herein, wherein,
R¹ is cyano and R⁹ is H;
R² is alkyl or alkoxyalkyl;
A₁ is —N—;
R¹⁰ is H;
R^{x} is alkyl, heteroarylalkyl or hydroxyalkyl, wherein the heteroaryl of the heteroarylalkyl is oxazolyl;
R^{y} is H or —OH;
and pharmaceutically acceptable salts thereof.

Particular examples of compounds of formula I as described herein are selected from
3-Hydroxy-5-methyl-4-[2-[[(3R)-1-(2-oxazol-2-ylethyl)-3-piperidyl]amino]oxazolo[4,5-b]pyridin-5-yl]benzonitrile;
3-Hydroxy-4-[2-[[(3R)-1-(2-hydroxyethyl)-3-piperidyl]amino]oxazolo[4,5-b]pyridin-5-yl]-5-(methoxymethyl)benzonitrile;
4-[2-[[(3R,5S)-1-Ethyl-5-hydroxy-3-piperidyl]amino]oxazolo[4,5-b]pyrazin-5-yl]-3-hydroxy-5-methyl-benzonitrile;2,2,2-trifluoroacetic acid;
3-Hydroxy-4-[2-[[(3R)-1-(2-hydroxyethyl)-3-piperidyl]amino]oxazolo[4,5-b]pyrazin-5-yl]-5-methyl-benzonitrile;2,2,2-trifluoroacetic acid;
and pharmaceutically acceptable salts thereof.

Other particular examples of compounds of formula I as described herein are selected from
4-[2-[[(3R,5S)-1-Ethyl-5-hydroxy-3-piperidyl]amino]oxazolo[4,5-b]pyrazin-5-yl]-3-hydroxy-5-methyl-benzonitrile;
3-Hydroxy-4-[2-[[(3R)-1-(2-hydroxyethyl)-3-piperidyl]amino]oxazolo[4,5-b]pyrazin-5-yl]-5-methyl-benzonitrile;
and pharmaceutically acceptable salts thereof.

Other particular examples of compounds of formula I as described herein are selected from
4-[(3S,5R)-3-Hydroxy-5-[[5-[2-hydroxy-6-methyl-4-(trifluoromethyl)phenyl]oxazolo[4,5-b]pyridin-2-yl]amino]-1-piperidyl]butanoic acid;
(3S,5R)-1-(2-Hydroxyethyl)-5-[[5-[2-hydroxy-6-methyl-4-(trifluoromethyl)phenyl]oxazolo[4,5-b]pyridin-2-yl]amino]piperidin-3-ol; 3-Hydroxy-4-[2-[[(3R,5S)-5-hydroxy-1-methyl-3-piperidyl]amino]oxazolo[4,5-b]pyrazin-5-yl]-5-methyl-benzonitrile;2,2,2-trifluoroacetic acid;
3-Hydroxy-4-[2-[[(3R)-1-(2-hydroxyethyl)-3-piperidyl]amino]-7-methoxy-oxazolo[4,5-b]pyridin-5-yl]-5-methyl-benzonitrile;2,2,2-trifluoroacetic acid;
3-Hydroxy-4-[2-[[(3R,5S)-5-hydroxy-3-piperidyl]amino]oxazolo[4,5-b]pyridin-5-yl]-5-methyl-benzonitrile;
3-Hydroxy-4-[2-[[(3R)-1-(2-hydroxyethyl)-3-piperidyl]amino]oxazolo[4,5-b]pyrazin-5-yl]-5-methyl-benzonitrile;
and pharmaceutically acceptable salts thereof.

Other particular examples of compounds of formula I as described herein are selected from
(3S,5R)-1-(2-Hydroxyethyl)-5-[[5-[2-hydroxy-6-methyl-4-(trifluoromethyl)phenyl]oxazolo[4,5-b]pyridin-2-yl]amino]piperidin-3-ol;
3-Hydroxy-4-[2-[[(3R,5S)-5-hydroxy-1-methyl-3-piperidyl]amino]oxazolo[4,5-b]pyrazin-5-yl]-5-methyl-benzonitrile;
3-Hydroxy-4-[2-[[(3R)-1-(2-hydroxyethyl)-3-piperidyl]amino]-7-methoxy-oxazolo[4,5-b]pyridin-5-yl]-5-methyl-benzonitrile;
and pharmaceutically acceptable salts thereof.

Other particular examples of compounds of formula I as described herein are selected from
5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyrazin-5-yl]-6-methyl-2,3-dihydrobenzofuran-4-ol;
5-[2-[[(3R)-1-(2-Hydroxyethyl)-3-piperidyl]amino]oxazolo[4,5-b]pyridin-5-yl]-6-methyl-2,3-dihydrobenzofuran-4-ol;
and pharmaceutically acceptable salts thereof.

Other particular examples of compounds of formula I as described herein are selected from
3-hydroxy-4-[2-[[(3R)-1-(2-hydroxyethyl)-3-piperidyl]amino]oxazolo[4,5-b]pyrazin-5-yl]-5-(methoxymethyl)benzonitrile;2,2,2-trifluoroacetic acid;
3-hydroxy-4-[2-[[(3R)-1-(2-hydroxyethyl)-3-piperidyl]amino]oxazolo[4,5-b]pyrazin-5-yl]-5-(methoxymethyl)benzonitrile;
3-hydroxy-4-[2-[[(3R)-1-(2-hydroxyethyl)-3-piperidyl]amino]-7-methoxy-oxazolo[4,5-b]pyridin-5-yl]-5-methyl-benzonitrile;
4-[2-[[(3R,5S)-1-ethyl-5-hydroxy-3-piperidyl]amino]oxazolo[4,5-b]pyrazin-5-yl]-3-hydroxy-5-methyl-benzonitrile;
5-[2-[[(3R)-1-(2-hydroxyethyl)-3-piperidyl]amino]oxazolo[4,5-b]pyrazin-5-yl]-6-methyl-indan-4-ol;
5-[2-[[(3R)-1-(2-hydroxyethyl)-3-piperidyl]amino]-7-methoxy-oxazolo[4,5-b]pyridin-5-yl]-6-methyl-indan-4-ol;
3-hydroxy-4-[2-[[(3R)-1-(2-hydroxyethyl)-3-piperidyl]amino]-7-methoxy-oxazolo[4,5-b]pyridin-5-yl]-5-(methoxymethyl)benzonitrile;
5-[2-[[(3R)-1-(2-hydroxyethyl)-3-piperidyl]amino]-7-methoxy-oxazolo[4,5-b]pyridin-5-yl]-6-(methoxymethyl)-2,3-dihydrobenzofuran-4-ol;
5-[2-[[(3R)-1-(2-hydroxyethyl)-3-piperidyl]amino]-7-methoxy-oxazolo[4,5-b]pyridin-5-yl]-6-methyl-2,3-dihydrobenzofuran-4-ol;
4-[2-[[(3R,5S)-1-ethyl-5-hydroxy-3-piperidyl]amino]oxazolo[4,5-b]pyrazin-5-yl]-3-hydroxy-5-(methoxymethyl)benzonitrile;2,2,2-trifluoroacetic acid;
4-[2-[[(3R,5S)-1-ethyl-5-hydroxy-3-piperidyl]amino]oxazolo[4,5-b]pyrazin-5-yl]-3-hydroxy-5-(methoxymethyl)benzonitrile;
(3S,5R)-1-(2-hydroxyethyl)-5-[[5-(4-hydroxy-6-methyl-2,3-dihydrobenzofuran-5-yl)oxazolo[4,5-b]pyrazin-2-yl]amino]piperidin-3-ol;
(3S,5R)-1-ethyl-5-[[5-(4-hydroxy-6-methyl-2,3-dihydrobenzofuran-5-yl)oxazolo[4,5-b]pyrazin-2-yl]amino]piperidin-3-ol;
3-hydroxy-4-[2-[[(3R)-1-(2-hydroxyethyl)-3-piperidyl]amino]oxazolo[4,5-b]pyrazin-5-yl]-5-(methoxymethyl)benzonitrile;
4-[2-[[(3R,5S)-1-ethyl-5-hydroxy-3-piperidyl]amino]-7-fluoro-oxazolo[4,5-b]pyridin-5-yl]-3-hydroxy-5-methyl-benzonitrile;
3-hydroxy-5-methyl-4-[2-[[(3R)-1-(3-oxazol-2-ylpropyl)-3-piperidyl]amino]oxazolo[4,5-b]pyridin-5-yl]benzonitrile;
4-[7-fluoro-2-[[(3R)-1-(2-hydroxyethyl)-3-piperidyl]amino]oxazolo[4,5-b]pyridin-5-yl]-3-hydroxy-5-methyl-benzonitrile;
4-[2-[[(3R,5S)-1-ethyl-5-hydroxy-3-piperidyl]amino]-7-methoxy-oxazolo[4,5-b]pyridin-5-yl]-3-hydroxy-5-methyl-benzonitrile;
(3S,5R)-1-ethyl-5-[[5-(4-hydroxy-6-methyl-2,3-dihydrobenzofuran-5-yl)oxazolo[4,5-b]pyridin-2-yl]amino]piperidin-3-ol;
and pharmaceutically acceptable salts thereof.

Preferred examples of compounds of formula I as described herein are selected from
4-[2-[[(3R,5S)-1-Ethyl-5-hydroxy-3-piperidyl]amino]oxazolo[4,5-b]pyrazin-5-yl]-3-hydroxy-5-methyl-benzonitrile;2,2,2-trifluoroacetic acid;
4-[2-[[(3R,5S)-1-Ethyl-5-hydroxy-3-piperidyl]amino]oxazolo[4,5-b]pyrazin-5-yl]-3-hydroxy-5-methyl-benzonitrile;
3-Hydroxy-4-[2-[[(3R)-1-(2-hydroxyethyl)-3-piperidyl]amino]oxazolo[4,5-b]pyrazin-5-yl]-5-methyl-benzonitrile;2,2,2-trifluoroacetic acid;
3-Hydroxy-4-[2-[[(3R)-1-(2-hydroxyethyl)-3-piperidyl]amino]oxazolo[4,5-b]pyrazin-5-yl]-5-methyl-benzonitrile;
5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyrazin-5-yl]-6-methyl-2,3-dihydrobenzofuran-4-ol;
5-[2-[[(3R)-1-(2-Hydroxyethyl)-3-piperidyl]amino]oxazolo[4,5-b]pyridin-5-yl]-6-methyl-2,3-dihydrobenzofuran-4-ol;
and pharmaceutically acceptable salts thereof.

Other preferred examples of compounds of formula I as described herein are selected from
4-[2-[[(3R,5S)-1-Ethyl-5-hydroxy-3-piperidyl]amino]oxazolo[4,5-b]pyrazin-5-yl]-3-hydroxy-5-methyl-benzonitrile; and
3-Hydroxy-4-[2-[[(3R)-1-(2-hydroxyethyl)-3-piperidyl]amino]oxazolo[4,5-b]pyrazin-5-yl]-5-methyl-benzonitrile.

More preferred examples of compounds of formula I as described herein are selected from
3-Hydroxy-4-[2-[[(3R)-1-(2-hydroxyethyl)-3-piperidyl]amino]oxazolo[4,5-b]pyrazin-5-yl]-5-methyl-benzonitrile;2,2,2-trifluoroacetic acid;
3-Hydroxy-4-[2-[[(3R)-1-(2-hydroxyethyl)-3-piperidyl]amino]oxazolo[4,5-b]pyrazin-5-yl]-5-methyl-benzonitrile;
5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyrazin-5-yl]-6-methyl-2,3-dihydrobenzofuran-4-ol;
5-[2-[[(3R)-1-(2-Hydroxyethyl)-3-piperidyl]amino]oxazolo[4,5-b]pyridin-5-yl]-6-methyl-2,3-dihydrobenzofuran-4-ol;
and pharmaceutically acceptable salts thereof.

Most preferred examples of compounds of formula I as described herein are selected from
5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyrazin-5-yl]-6-methyl-2,3-dihydrobenzofuran-4-ol;
5-[2-[[(3R)-1-(2-Hydroxyethyl)-3-piperidyl]amino]oxazolo[4,5-b]pyridin-5-yl]-6-methyl-2,3-dihydrobenzofuran-4-ol;
and pharmaceutically acceptable salts thereof.

A most preferred example of a compound of formula I as described herein is 5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol, or a pharmaceutically acceptable salt thereof.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a metabolic disease;
(vi) a cardiovascular disease;
(vii) a respiratory disease;
(viii) a liver disease;
(ix) a renal disease;
(x) an ocular disease;
(xi) a skin disease;
(xii) a lymphatic condition;
(xiii) graft versus host disease;
(xiv) allodynia;
(xv) a condition associated with diabetes; and
(xvi) any disease where an individual has been determined to carry a germline or somatic non-silent mutation in NLRP3

In another embodiment, the disease, disorder or condition is selected from:
(i) cancer;
(ii) an infection;
(iii) a cardiovascular disease;
(iv) a liver disease;
(v) an ocular disease; and
(vi) a skin disease.

In a further typical embodiment of the invention, the disease, disorder or condition is inflammation. Examples of inflammation that may be treated or prevented include inflammatory responses occurring in connection with, or as a result of
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia; (vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(x) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xi) a lymphatic condition such as Castleman's disease;
(xii) a condition of, or involving, the immune system, such as hyper IgE syndrome, lepromatous leprosy, familial hemophagocytic lymphohistiocytosis, or graft versus host disease;
(xiii) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xiv) a cancer, including those cancers listed above;
(xv) a burn, wound, trauma, haemorrhage or stroke;
(xvi) radiation exposure;
(xvii) a metabolic disease such as type 2 diabetes (T2D), atherosclerosis, obesity, gout or pseudo-gout; and/or
(xiii) pain such as inflammatory hyperalgesia, pelvic pain, allodynia, neuropathic pain, or cancer-induced bone pain.

An embodiment of the present invention is a compound according to formula I as described herein for the treatment or prophylaxis of a disease, disorder or condition selected from:
inflammation;
an auto-immune disease;
cancer;
an infection;
a metabolic disease;
a cardiovascular disease;
a respiratory disease;
a liver disease;
a renal disease;
an ocular disease;
a skin disease;
a lymphatic condition;
graft versus host disease;
allodynia;
a condition associated with diabetes; and
any disease where an individual has been determined to carry a germline or somatic non-silent mutation in NLRP3.

An embodiment of the present invention is the use of a compound according to formula I as described herein in the treatment or prophylaxis of a disease, disorder or condition, wherein the disease, disorder or condition is responsive to NLRP3 inhibition.

An embodiment of the present invention is the use a compound according to formula I as described herein for use in the treatment or prophylaxis of a disease, disorder or condition selected from Asthma and COPD.

An embodiment of the present invention is the use a compound according to formula I as described herein for use in the treatment or prophylaxis of a cardiovascular disease, disorder or condition.

An embodiment of the present invention is the use a compound according to formula I as described herein for use in the treatment or prophylaxis of a cardiometabolic disease, disorder or condition.

An embodiment of the present invention is the use a compound according to formula I as described herein for use in the treatment or prophylaxis of a disease, disorder or condition selected from Cryopyrin-associated periodic syndromes.

An embodiment of the present invention is a compound according to formula I as described herein for the treatment or prophylaxis of a disease, disorder or condition selected from Asthma and COPD.

An embodiment of the present invention is a compound according to formula I as described herein for the treatment or prophylaxis of a cardiovascular disease, disorder or condition.

An embodiment of the present invention is a compound according to formula I as described herein for the treatment or prophylaxis of a cardiometabolic disease, disorder or condition.

An embodiment of the present invention is a compound according to formula I as described herein for the treatment or prophylaxis of a disease, disorder or condition selected from Cryopyrin-associated periodic syndromes.

An embodiment of the present invention is the use of a compound according to formula I as described herein for the preparation of a medicament for the treatment or prophylaxis of a disease, disorder or condition selected from Asthma and COPD.

An embodiment of the present invention is the use of a compound according to formula I as described herein for the preparation of a medicament for the treatment or prophylaxis of a cardiovascular disease, disorder or condition.

An embodiment of the present invention is the use of a compound according to formula I as described herein for the preparation of a medicament for the treatment or prophylaxis of a cardiometabolic disease, disorder or condition.

An embodiment of the present invention is the use of a compound according to formula I as described herein for the preparation of a medicament for the treatment or prophylaxis of a disease, disorder or condition selected from Cryopyrin-associated periodic syndromes.

An embodiment of the present invention is a method of treatment or prophylaxis of a disease, disorder or condition selected from Asthma and COPD, which method comprises administering an effective amount of a compound according to formula I as described herein.

An embodiment of the present invention is a method of treatment or prophylaxis of a cardiovascular disease, disorder or condition, which method comprises administering an effective amount of a compound according to formula I as described herein.

An embodiment of the present invention is a method of treatment or prophylaxis of a cardiometabolic disease, disorder or condition, which method comprises administering an effective amount of a compound according to formula I as described herein.

An embodiment of the present invention is a method of treatment or prophylaxis of a disease, disorder or condition selected from Cryopyrin-associated periodic syndromes, which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1P) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 pg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.

Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)

Add 5 μl compound (8 points half-log dilution, with 10 μM top dose) or vehicle (DMSO 0.1% FAC) to the appropriate wells

Incubate for 3 hours at 37° C., 5% CO₂

Add 5 μl nigericin (Sigma #N7143) (FAC 5 μM) to all wells

Incubate for 1 hr at 37° C., 5% CO₂

At the end of the incubation period, spin plates at 300 xg for 3 mins and remove supernatant

Then add 50 μl of resazurin (Sigma #R7017) (FAC 100 μM resazurin in RPMI medium without FBS) and incubate plates for a further 1-2 hours at 37° C. and 5% CO₂

Plates were read in an Envision reader at Ex 560 nm and Em 590 nm

IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.

Plate out 80 μl of whole blood containing 1p g/ml of LPS in 96-well, clear bottom cell culture plate (Corning #3585)

Add 10 μl compound (8 points half-log dilution with 10 μM top dose) or vehicle (DMSO 0.1% FAC) to the appropriate wells

Incubate for 3 hours at 37° C., 5% CO₂

Add 10 μl nigericin (Sigma #N7143) (10 μM FAC) to all wells

Incubate for 1 hr at 37° C., 5% CO₂

At the end of the incubation period, spin plates at 300 xg for 5 mins to pellet cells and remove 20 μl of supernatant and add to 96-well v-bottom plates for IL-1β analysis (note: these plates containing the supernatants can be stored at −80° C. to be analysed at a later date)

IL-1β was measured according to the manufacturer protocol (Perkin Elmer-AlphaLisa IL-1 Kit AL220F-5000)

IC₅₀ data is fitted to a non-linear regression equation (log inhibitor vs response-variable slope 4-parameters)

The results of the human whole blood assay are summarised in Table 1 below as HWB IC₅₀.

### Microsomal Stability:

Incubations of test compounds at 1 μM in microsomes (0.5 mg/mL) plus cofactor NADPH are performed in 96 well plates at 37° C. on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

Assay descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures; pooled (n=5 for male and n=5 for female for human)] with stromal mouse fibroblasts (negative control; pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 mg/mL hydrocortisone and 0.004 mg/mL insulin, to a final suspension density of 1×106 cells/mL. The incubation was performed fully automatically with Liquid Handling System (Tecan) equipped with a CO₂ incubator with an orbital shaker. After the addition of a test compound at e.g. 1 μM to the wells (1×105 cells/well), the 96-well hepatocyte suspension culture plates are incubated in a 5% CO₂ at 37° C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO₂ atmosphere and 37° C.). The incubation media in human HepatoPac® is identical with that in suspended hepatocytes. At defined time points (2, 18, 26, 48, 72 and 96 h), whole wells are quenched with ice-cold acetonitrile containing an internal standard.

Samples are then centrifuged appropriately and the supernatant analyzed by LC-MS/MS. The incubation is conducted in n=1 or 2.

hERG Screening Assay

In the drug development process of small molecules, one of the most frequent adverse side effects, leading to the failure of drugs, is the cardiac arrhythmias. Such failure is often related to the capacity of the drug to inhibit the human ether-à-go-go-related gene (hERG) cardiac potassium channel. Having no or low inhibition of the hERG cardiac potassium channel is therefore considered as beneficial.

### Cells

The CHO crelox hERG cell line (ATCC reference Nr. PTA-6812, female Chinese hamster cells) was generated and validated at Roche. Ready-to-use frozen instant CHO-hERG cells were cryopreserved at Evotec (Germany) and used directly in the experiments.

### Experimental Solutions

The extracellular solution contains (in mM): NaCl 150; KCl 4; CaCl₂ 1; MgCl₂ 1; HEPES 10; pH 7.2-7.4 with NaOH, osmolarity 290-330 mOsm. The internal solution contains (in mM): KCl, 10; KF, 100; NaCl, 10; HEPES, 10; EGTA, 20; pH=7.0-7.4 with KOH, osmolarity 260-300 mOsm.

### Electrophysiology

The effects of a compound on hERG K+-currents parameters will be evaluated at 2 concentrations in at least 4 cells.

The hERG test is performed using automated patch clamp system SynchroPatch® 384 (Nanion Technologies GmbH, Germany). K+ currents are measured with the patch-voltage-clamp technique in the whole-cell configuration at 35-37° C.

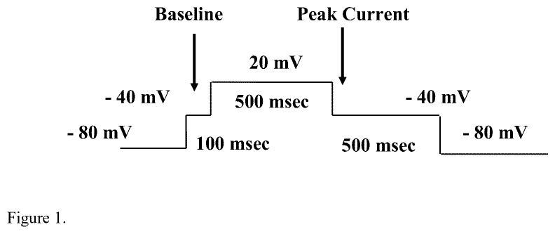

Cells were held at a resting voltage of −80 mV and they were stimulated by a voltage pattern shown in FIG. 1 (pulse pattern used to elicit outward K) current at 35-37° C.) to activate hERG channels and conduct outward IKhERG current, at a stimulation frequency of 0.1 Hz (6 bpm)

### Data Analysis

The amplitudes of IKhERG were recorded in each concentration of drug and they were compared to the vehicle control values (taken as 100%) to define fractional blocks. The concentration-response data were fitted with the following relationship:

| | $I ( C ) = \frac{100}{1 + {( C / IC 50 )}^{h}}$ |
|---|---|
| where | C is the concentration, |
| | IC₅₀ is the concentration producing 50% block |
| | h is the Hill coefficient. |

Concentration-response curves were fitted by non-linear regression analysis using EworkBook suite (ID Business Solutions Ltd, UK). Data fit was done with the 4 Parameter Logistic Model (fit=(A+(B/(1+((x/C)\hat{ }D)))), where A=0 and B=100).

The results of the hERG assay are summarised in Table 2 below as hERG IC₂₀.

### Transcellular P-gp Assay:

The general assay uses transfected LLC-PK1 cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer. The tightness of the cell monolayer and functional activity of P-gp are confirmed by addition of a cell-impermeable marker, Lucifer yellow, and a reference P-gp substrate, edoxaban, respectively. J. Pharmacol. Exp Ther., 2021, 376, 322-329.

### PAMPA:

PAMPA (Parallel Artificial Membrane Permeability Assay) is a first line permeability screen for drug candidates. The PAMPA assay mimics the transcellular absorption conditions using an artificial phospholipid membrane. This assay determines a permeability value that can be used for compound optimization and ranking purposes as well as input parameters for in silico models to predict intestinal absorption.

The donor concentration is measured at t-start (reference) and compared with the donor and acceptor concentration after a certain time (t-end) to calculate the extent of passage of the compound through the membrane.

### Bacterial Reverse Mutation Test (AMES):

The testing of compounds is conducted as outlined in this guideline: Test No. 471: Bacterial Reverse Mutation Test | OECD Guidelines for the Testing of Chemicals, Section 4: Health Effects | OECD iLibrary (oecd-ilibrary.org)

### Bacteria Culture:

The bacterial strains used are TA98, TA100, TA1535, TA97a and TA102. Batches of each strain, are maintained as frozen stocks. Vials are thawed and used to inoculate cultures in nutrient broth.

The cultures are placed in an incubator set to 37° C. with agitation for approximately 10 hours to provide a working culture of at least 108 cells per mL.

To ensure cultures are at the appropriate phase of growth and culture density, a sample is taken from each culture at the end of the incubation period and assessed for culture density by either viability plating or OD650 assessment.

### Treatment:

3 replicates per concentration of compound and positive controls and 6 replicates per vehicle controls are included.

Formulations are prepared using DMSO to allow maximum exposure up to the solubility limit or 1000 μg/well for a freely soluble test article. This concentration is equivalent to 5000 μg/plate as used in the usual plate incorporation Ames assay.

Concentrations are usually separated by half-log intervals in a single experiment. For soluble compounds, concentrations will be 0, 3.2, 10, 32, 100, 320, 1000 μg/well.

### Positive Controls Used are:

| Abbreviation | Name | Used for strain |
|---|---|---|
| 2NF | 2-Nitrofluorene | TA98 − S-9 |
| NaN3 | Sodium Azide | TA100 and TA1535 − S-9 |
| AAC | 9-Aminoacridine | TA97a − S-9 |
| MMC | Mitomycin C | TA102 − S-9 |
| B[a]P | Benzo[a]pyrene | TA98 + S-9 |
| AAN | Aminoanthracene | TA100, TA1535, TA97a |
| | | and TA102 + S-9 |

Platings will be achieved by the following sequence of additions to 400 μL supplemented molten agar at 45±1° C.:
20 μL of bacterial culture
20 μL of test article solution/vehicle control/positive control
100 μL of 10% S-9 mix or buffer solution
   followed by rapid mixing and pouring onto mutation plates (wells).

When set, the plates will be inverted and incubated protected from light for 2 to 3 days in an incubator set to 37° C.

### Toxicity:

Toxicity is detected by the following parameters:
Diminution of background lawn
Marked reduction in revertants compared to the concurrent vehicle controls
Reduction in mutagenic response.

Scoring: Scoring of bacteria colonies is performed manually or electronically using automated colony counter.

### In Vitro Mammalian Cell Micronucleus Test:

The testing of compounds is conducted as outlined in this guideline: Test No. 487: In Vitro Mammalian Cell Micronucleus Test | OECD Guidelines for the Testing of Chemicals, Section 4: Health Effects | OECD iLibrary (oecd-ilibrary.org)

### Cell Culture:

Cultures are maintained in tissue culture flasks containing HEPES-buffered RPMI 1640 medium with GlutaMAX-1 including 10% (v/v) heat inactivated foetal calf serum, 100 Units/mL/100 μg/mL penicillin/streptomycin in a humidified incubator set to 37° C., 5% (v/v) CO₂ in air. Cells will be subcultured at low to medium density at least once prior to treatment.

On the day prior to treatment, cells will be subcultured at a density of approximately 7×104 cells/mL. Cells will be maintained at 37° C., 5% (v/v) CO₂ in air, in a humidified environment prior to treatment.

### Treatment:

Cultured human lymphoblastoid TK6 cells will be exposed to the compound for 3 hours in the presence of S-9, followed by a recovery period of 24 hours. In addition, a continuous 27 hour treatment in the absence of S-9 will be included as a number of chemicals have been reported as only exerting positive effects following prolonged treatment. This is equivalent to approximately 1.5-2.0 times the average generation time of the TK6 cells used in this laboratory (cell cycle approximately 15 hours). All cultures will be sampled 27 hours after the beginning of treatment. Dilutions will be prepared in DMSO that allow maximum exposure up to the solubility limit, 1 mM or 500 μg/mL, whichever is lower.

Normally, at least 12 concentrations separated by 0.7-fold intervals, ranging down from the upper limit (for soluble compounds with a MW≥500, concentrations will be 9.887, 14.12, 20.18, 28.82, 41.18, 58.82, 84.04, 120.1, 171.5, 245, 350 and 500 μg/mL). The final concentration of DMSO will be 1% v/v. Positive controls are Noscapine in the absence of S-9 and Cyclophosphamide in the presence of S-9. 2 replicates per concentration of compound and multiple concurrent vehicle and positive controls will be included per treatment in 96-well plates and incubated for the treatment time at 37° C., 5% (v/v) CO₂. 3 hour treatment cultures will be washed once and reincubated with fresh medium for 24 hours.

### Harvesting:

At the defined sampling time an aliquot of cell suspension from designated cultures will be taken for determination of cell number by using a Coulter Counter. Cultures designated for analysis will be centrifuged at approximately 200 g, 5 minutes. Cells will be resuspended in 0.075 M KCl prior to fixation in fresh, cold methanol/glacial acetic acid (7:1 v/v). Fixed cells will be stored in fixative at 2-8° C. prior to slide preparation. Slides will be air-dried prior to staining by immersion in 12.5 μg/mL Acridine Orange in phosphate buffered saline (PBS), pH 6.8 for approximately 10 minutes, following by a wash with PBS (with agitation) for a few seconds.

### Cytotoxicity Readout and Selection of Concentrations:

Toxicity is expressed as Population Doubling (PD) relative to vehicle controls. PD will be calculated for each concentration as follows:

$$PD = [ log ( N / {X}_{0} ) ] / log 2$$

$$Where N = mean final cell count / culture at each concentration$$

$${X}_{0} = starting ( baseline ) count$$

The highest concentration for micronucleus analysis should either not exceed (approximately) 50% cytotoxicity, be the highest concentration tested, or, be the lowest precipitating concentration observed by eye at the end of the treatment incubation period. Slides from the highest selected concentration and at least two lower concentrations will be analysed, such that a range of cytotoxicity from maximum to little or none is covered, where appropriate. A minimum of 1000 mononucleate cells from each culture (2000 per concentration) will be analysed for micronuclei.

### Evaluation Criteria:

The compound will be considered to induce clastogenic and/or aneugenic events if:
A statistically significant increase in the frequency of MNMON cells at one or more concentrations is observed.
The incidence of cells with micronuclei at such a concentration exceeds the normal range in both replicates.
A concentration-related increase in the proportion of cells with micronuclei is observed (positive trend test).

The compound will be considered positive in this assay if all of the above criteria are met.

The compound will be considered negative in this assay if none of the above criteria are met.

Results which only partially satisfy the above criteria will be dealt with on a case-by-case basis, but in the context of the screening study, will be concluded as either positive, negative or equivocal. Evidence of a concentration-related effect is considered useful but not essential in the evaluation of a positive result. Biological relevance will be taken into account, for example consistency of response within and between concentrations and (if applicable) between experiments, or effects occurring only at very toxic concentrations.$

### Pharmacokinetics Profile of Test Substances in Minipigs:

The pharmacokinetics of the test substance was determined in minipigs following intravenous and oral administration. The experimental design consisted of three male minipigs, of which each animal received a single intravenous bolus dose, and a single oral doses with the test item. Intravenous doses were administered at a nominal dose volume of 1 mL/kg. Oral doses were administered by gavage at a nominal dose volume of 5 mL/kg. There was a washout period of at least 7 days between last sampling occasion and the next dosing occasion to the same animal. The content of all formulations was within the desired range of 85 to 115% of the nominal content. Following dosing, samples of blood (1 mL) were withdrawn from the saphenous (via cannula) or jugular vein of each animal at pre-dose, 5, 15, 30 min, 1, 2, 4, 8, 24 hours post-dose after IV dosing and at pre-dose, 15, 30 min, 1, 2, 4, 6, 8, 24, 48 hours post-dose after oral dosing. At all time points, the haematocrit was determined. The blood:plasma partitioning factor was determined at the 2 and 4 hour time point, and urine was collected as a single sample for 24 hours after dose administration.

Blood samples (nominally 1 mL) were withdrawn from the saphenous (via cannula) or jugular vein of each animal into polypropylene tubes containing K2EDTAanticoagulant and was centrifuged (1500 g, 10 min, 4° C.) to prepare plasma for analysis. Residual blood cells were discarded. Plasma vials were capped and stored on wet-ice for no longer than 60 minutes before being transferred to <−50° C. storage (nominally −80° C.) prior to analysis with a specific LC-MS method.

### Toxicity Assessment of Test Substance in Minipig

The maximum tolerated dose (MTD) of the test item is determined following once daily oral (gavage) administration to the minipig. The toxicity of repeated daily administration for 14 days is then assessed. In addition, the toxicokinetic profile of the test item is characterized. Sufficient purpose-bred Gottingen minipigs are obtained from Ellegaard Göttingen, Dalmose, Denmark (Animals: 2 to 3 month age range and in a 4 to 6 kg weight range). At start of dosing animals are 4 to 5 months old and in a 6 to 9.5 kg weight range. A dose volume of 10 mL/kg is used. Individual dose volumes are calculated from the most recent body weights for each animal to target dose levels of 30, 100 and 300 mg/kg/day or others depending non MTD results. Blood samples are taken on day 1 and day 14 for the determination of drug concentration in plasma and derived toxicokinetic parameters. Animals are not fed on the day of scheduled necropsy. Each animal is anaesthetized via intramuscular injection of a Zoletil mix then killed by exsanguination. All tissues are preserved in the appropriate fixative/s. Further analysis includes food consumption, body weight, clinical pathology, and full histopathological examination of target organs.

TABLE 1
NLRP3 inhibitory activity

| | THP-1 | Human |
|---|---|---|
| | pyroptosis | whole blood |
| Example | Assay | IL-1β Assay |
| No. | IC₅₀ (nM) | IC₅₀ (nM) |
| 1 | 3.3 | 24.2 |
| 2 | 4.3 | 19.6 |
| 3 | 27.9 | 35.2 |
| 4 | 26.1 | 192.3 |
| 5 | 51 | 432 |
| 6 | 12 | 95 |
| 7 | 10 | 17 |
| 8 | 11 | 28 |
| 9 | 45 | 84 |
| 10 | 5 | 29 |
| 11 | 4 | 8 |
| 12 | 8 | 15 |
| 13 | 0.8 | 19 |
| 14 | 0.6 | 22 |
| 15 | 34 | 33 |
| 16 | 8 | 22 |
| 17 | 3 | 18 |
| 18 | 2 | |
| 19 | 4 | |
| 20 | 75 | 68 |
| 21 | 9 | |
| 22 | 5 | 39 |
| 23 | 44 | 34 |
| 24 | 19 | 53 |
| 25 | 2 | 16 |
| 27 | 6 | 95 |
| 28 | 23 | 86 |
| 29 | 7 | 124 |
| 30 | 13 | 48 |

TABLE 2
Transcellular human P-gp Assay:

| Example | Permeability | |
|---|---|---|
| No. | [nm/sec] | ER |
| 1 | 26 | 4.4 |
| 2 | 29 | 3.0 |
| 3 | 19 | 2.9 |
| 11 | 26 | 3.3 |
| 13 | 83 | 3.5 |
| 14 | 135 | 4.0 |
| 16 | 37 | 5 |
| 22 | 122 | 9 |
| 31 | 112 | 3 |

* ER = efflux ratio

TABLE 3
hERG inhibitory activity

| Example | hERG IC₂₀ |
|---|---|
| No. | [μM] |
| 1 | >10 |
| 3 | 6 |
| 11 | >10 |
| 12 | >10 |
| 13 | >10 |
| 15 | >10 |
| 16 | >10 |
| 17 | >10 |
| 18 | 6.3 |

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization. The salt free compounds can be made analogues to the described examples yielding salts.

### Experimental Methods

Abbreviations

| | |
|---|---|
| ACN | Acetonitrile |
| Aq. | Aqueous |
| DCE | Dichloroethane |
| DCM | Dichloromethane |
| DIEA | N,N-Diisopropylethylamine |
| DMF | Dimethylformamide |
| ESI | Electrospray ionization |
| EtOH | Ethanol |
| EtOAC | Ethyl acetate |
| eq | Equivalent |
| h, hrs | Hour(s) |
| HPLC | High-performance liquid chromatography |
| LCMS | Liquid chromatography-mass spectrometry |
| MeCN | Acetonitrile |
| MeOH | Methanol |
| mins | Minutes |
| MTBE | Methyl tert-butyl ether |
| PE | Petroleum ether |
| rt | Room temperature |
| TEA | Triethylamine |
| TBAF | Tetrabutylammoniumfluorid |
| THF | Tetrahydrofuran |
| TFA | Trifluoroacetic acid |
| sat | Saturated |
| SFC | Supercritical fluid chromatography |

### Preparation of Intermediates

### Intermediate 1

### 3-Methyl-4-(4,4,5,5-tetramethyl-1,3,2-dioxaborolan-2-yl)-5-(2-trimethylsilylethoxymethoxy)benzonitrile

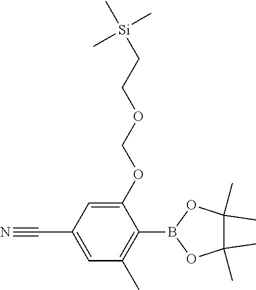 [SMILES: Cc1cc(C#N)cc(OCOCC[Si](C)(C)C)c1B1OC(C)(C)C(C)(C)O1]

### Step A: 4-Bromo-2-methoxy-6-methyl-aniline

To a solution of 2-methoxy-6-methylaniline (CAS #50868-73-0, 50.0 g, 364.5 mmol, 1.0 eq) in methanol (150 mL) and acetic acid (50.0 mL, 874.2 mmol, 2.4 eq) Br₂ (22.4 mL, 437.4 mmol, 1.2 eq) was added slowly dropwise under 0° C. Afterwards the mixture was stirred at 25° C. for 2 h. Upon reaction completion, the above reaction solution was diluted with water (300 mL) and extracted with ethyl acetate (3×100 mL). The combined organic phase was washed with brine (100 mL), dried over anhydrous sodium sulfate, and filtered and the filtrate was concentrated under reduced pressure and purified over column chromatography on silica gel (PE/EtOAc, 1:0 to 10:1) to afford the title compound (30.3 g, 38% yield) as a brown solid. ¹H NMR (400 MHz, DMSO-d₆) δ [ppm]: 6.82 (d, 1H), 6.78 (d, 1H), 4.57 (m, 2H), 3.77 (s, 3H), 2.06 (s, 3H).

### Step B: 4-Amino-3-methoxy-5-methylbenzonitrile

To a solution of 4-bromo-2-methoxy-6-methyl-aniline (30.3 g, 140.2 mmol, 1.0 eq) and Zn(CN)₂ (16.5 g, 140.51 mmol, 1.0 eq) in DMF (260 mL) was added Pd(PPh₃)₄ (32.4 g, 28.1 mmol, 0.2 eq) under N₂. The mixture was stirred at 100° C. for 12 h under a nitrogen atmosphere. Upon reaction completion, the mixture was cooled to 20° C., poured into water (500 mL) and extracted with ethyl acetate (3×300 mL). The organic phase was washed with brine (3×200 mL), dried over Na₂SO₄, filtered, and the filtrate was concentrated under reduced pressure. The crude was purified over column chromatography on silica gel (PE/EtOAc, 1:0 to 5:1) to afford the title compound (22.0 g, 97% yield) as a pink solid. ¹H NMR (400 MHz, DMSO-d₆) δ [ppm]: 7.05 (s, 2H), 5.48 (s, 2H), 3.81 (s, 3H), 2.09 (s, 3H).

### Step C: 4-Bromo-3-methoxy-5-methylbenzonitrile

To a solution of 4-amino-3-methoxy-5-methylbenzonitrile (22.0 g, 135.6 mmol, 1.0 eq) and CuBr (29.2 g, 203.5 mmol, 1.5 eq) in MeCN (220 mL) butyl nitrite (20.9 mL, 203.5 mmol, 1.5 eq) was added dropwise at 25° C. Then the mixture was stirred at 65° C. for 2 h. Upon reaction completion, the mixture was cooled to 20° C., diluted with MeCN (150 mL), filtered and the filtrate was concentrated under reduced pressure and purified over column chromatography on silica gel (hexane/EtOAc, 1:0 to 5:1). Afterwards the residue was slurried with a mixture of ethyl acetate (30 mL) and petroleum ether (3 mL), filtered and the cake was collected to afford the title compound (18.0 g, 59% yield) as a white solid. ¹H NMR (400 MHz, DMSO-d₆) δ [ppm]: 7.46 (s, 1H), 7.43 (s, 1H), 3.91 (s, 3H), 2.39 (s, 3H).

### Step D: 4-Bromo-3-hydroxy-5-methylbenzonitrile

To a solution of 4-bromo-3-methoxy-5-methylbenzonitrile (6.0 g, 26.5 mmol, 1.0 eq) in DCM (30 mL) was added BBr₃ (30.0 mL, 316.1 mmol, 11.9 eq) at 0° C., then stirred at 25° C. for 1 h. Upon reaction completion, it was combined with another batch (10 g) and diluted with water (500 mL). The mixture was extracted with ethyl acetate (3×150 mL) and the combined organic phase was washed with brine (200 mL), dried over anhydrous sodium sulfate, filtered, and the filtrate was concentrated under reduced pressure and purified over column chromatography on silica gel (hexane/EtOAc, 1:0 to 10:1) to afford the title compound (13.3 g, 89% yield) as a yellow solid. ¹H NMR (400 MHz, DMSO-d₆) δ [ppm]: 10.15 (s, 1H), 6.42 (d, 1H), 6.24 (d, 1H), 1.51 (s, 3H).

### Step E: 4-Bromo-3-methyl-5-(2-trimethylsilylethoxymethoxy)benzonitrile

To a solution of 4-bromo-3-hydroxy-5-methylbenzonitrile (13.3 g, 62.7 mmol, 1.0 eq) in DMF (60 mL) was added Cs₂CO₃ (33.3 g, 102.1 mmol, 1.6 eq) at 0° C., then stirring was continued at 20° C. for 0.5 h. Afterwards, 2-(trimethylsilyl)ethoxymethyl chloride (CAS #76513-69-4, 15.5 mL, 87.8 mmol, 1.4 eq) was added dropwise at 0° C., then stirred at 20° C. for 1 h. Upon reaction completion, water (200 mL) was added and the mixture was extracted with MTBE (3×150 mL). The combined organic phase was washed with brine (150 mL), dried over anhydrous sodium sulfate, filtered and the filtrate was concentrated reduced pressure and purified over column chromatography on silica gel (hexane/EtOAc, 1:0 to 10:1) to afford the title compound (16.3 g, 76% yield) as a white solid. ¹H NMR (400 MHz, CDCl₃) δ [ppm]: 7.27 (d, 1H), 7.18 (d, 1H), 5.30 (s, 2H), 3.77 (t, 2H), 2.44 (s, 3H), 0.95 (t, 2H), 0.00 (s, 9H).

### Step F: 3-Methyl-4-(4,4,5,5-tetramethyl-1,3,2-dioxaborolan-2-yl)-5-(2-trimethylsilylethoxy-methoxy)benzonitrile

To a solution of 4-bromo-3-methyl-5-(2-trimethylsilylethoxymethoxy)benzonitrile (10.0 g, 29.2 mmol, 1.0 eq) and 4,4,4′,4′,5,5,5′,5′-octamethyl-2,2′-bi(1,3,2-dioxaborolane) (14.8 g, 58.43 mmol, 2.0 eq) in 1,4-dioxane (100 mL) was added Cs₂CO₃ (19.04 g, 58.43 mmol, 2.0 eq), tris(4-methoxy-3,5-dimethylphenyl)phosphane (CAS #121898-64-4, 1.28 g, 2.92 mmol, 0.1 eq), Pd(OAc)₂ (0.66 g, 2.92 mmol, 0.1 eq) and the mixture was stirred at 95° C. for 5 h under N₂. Upon reaction completion, the mixture was cooled to r.t., diluted with water (150 mL) and ethyl acetate (50 mL), filtered and the filtrate was extracted with ethyl acetate (3×100 mL). The combined organic phase was washed with brine (50 mL), dried over anhydrous sodium sulfate, filtered, the filtrate was concentrated under reduced pressure and purified over column chromatography on silica gel (PE/EtOAc, 1:0 to 10:1) (17 g), then triturated with MeOH (51 mL), filtered and the cake was collected to afford the title compound (8.38 g, 74% yield) as white solid. ¹H NMR (400 MHz, CDCl₃) δ [ppm]: 7.13 (s, 1H), 7.07 (s, 1H), 5.18 (s, 2H), 3.72 (t, 2H), 2.36 (s, 3H), 1.38 (s, 12H), 0.94 (t, 2H), 0.00 (s, 9H).

### Intermediate 2

### 4-(6-Amino-5-benzyloxy-pyrazin-2-yl)-3-methyl-5-(2-trimethylsilylethoxymethoxy)-benzonitrile

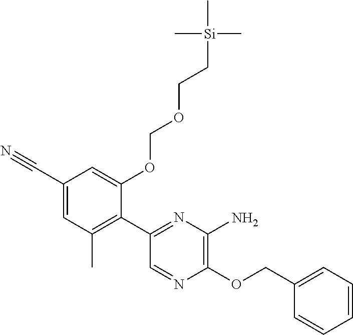 [SMILES: Cc1cc(C#N)cc(OCOCC[Si](C)(C)C)c1-c1cnc(OCc2ccccc2)c(N)n1]

### Step A: 3-Benzyloxy-6-chloro-pyrazin-2-amine

To a stirred solution of benzyl alcohol (4.49 mL, 43.2 mmol, 1.0 eq) in THF (90 mL) at room temperature was added NaH (1.73 g, 43.18 mmol, 1.0 eq 60% in mineral oil) and stirring was continued for 10 minutes. Then 3-bromo-6-chloropyrazin-2-amine (CAS #212779-21-0, 9.0 g, 43.2 mmol, 1.0 eq) was added and the reaction mixture was heated at 70° C. for 4 h. The reaction mixture was cooled to room temperature. Ethyl acetate (60 mL) and water (80 mL) were added and layers were separated. The aqueous phase was extracted with ethyl acetate (80 mL×2). Combined extracts were washed with brine (80 mL), dried over Na₂SO₄, filtered, and concentrated under reduced pressure. The residue was purified by column chromatography on silica gel (petroleum ether:ethyl acetate=5:1 to 1:1), then purified by normal phase flash (column: Welch Ultimate XB-CN 250*70*10 um; condition: hexane-EtOH Begin B 12 End B 12; gradient time (min): 15 100% B; hold time (min) 5; flow rate (ml/min) 140) to afford the title compound (2.3 g, 23% yield) as a yellow solid. LCMS: m/z 236.0 [M+H]⁺, ESI pos.

### Step B: 4-(6-Amino-5-benzyloxy-pyrazin-2-yl)-3-methyl-5-(2-trimethylsilylethoxymethoxy)-benzonitrile

To a solution of aforementioned 3-benzyloxy-6-chloro-pyrazin-2-amine (2000 mg, 8.49 mmol, 1.0 eq) and 3-methyl-4-(4,4,5,5-tetramethyl-1,3,2-dioxaborolan-2-yl)-5-(2-trimethylsilylethoxy-methoxy)-benzonitrile (3965.2 mg, 10.2 mmol, 1.2 eq), CsF (2578.2 mg, 16.9 mmol, 2.0 eq) in 1,4-dioxane (30 mL) and water (3 mL) was added XPhos Pd G₃ (719.2 mg, 0.85 mmol, 0.1 eq) under N₂. Then the mixture was stirred at 95° C. for 2 h. Upon the reaction completion, the reaction mixture was cooled to room temperature. Ethyl acetate (150 mL) and water (150 mL) were added and layers were separated. The aqueous phase was extracted with ethyl acetate (150 mL×2). Combined extracts were washed with brine (250 mL), dried over Na₂SO₄, filtered, and concentrated under vacuum to give a residue. The residue was purified by column chromatography on silica gel (petroleum ether:ethyl acetate=2:1 to 1:1) to give the title compound (2.70 g, 69% yield) as a yellow solid. LCMS: m/z 463.2 [M+H]⁺, ESI pos.

### Intermediate 3

### (3R)-1-[2-[tert-Butyl(dimethyl)silyl]oxyethyl]piperidin-3-amine

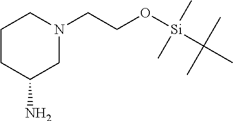 [SMILES: CC(C)(C)[Si](C)(C)OCCN1CCC[C@@H](N)C1]

### Step A: Benzyl N-[(3R)-1-[2-[tert-butyl(dimethyl)silyl]oxyethyl]-3-piperidyl]carbamate

To a solution of benzyl N-[(3R)-3-piperidyl]carbamate (CAS #478646-32-1, 2.0 g, 8.54 mmol, 1.0 eq) in methanol (30 mL) was added (tert-butyldimethylsilyloxy)acetaldehyde (1.64 g, 9.39 mmol, 1.1 eq), followed by NaBH₃CN (1.35 g, 21.3 mmol, 2.5 eq). The mixture was stirred at 20° C. for 3 hours. The mixture was quenched with water (50 mL) and then concentrated under vacuum to remove methanol. Afterwards, EtOAc (100 mL) was added and layers were separated. The aqueous phase was extracted with EtOAc (300 mL×2). The combined extracts were washed with brine (50 mL), dried over Na₂SO₄, filtered, and concentrated under reduced pressure. The residue was purified by column chromatography on silica gel (PE/EtOAc; PE=77% to 33%) to give the title compound (2100.0 mg, 63% yield) as colorless oil. LCMS m/z 393.3 [M+H]⁺, ESI pos.

### Step B: (3R)-1-[2-[tert-Butyl(dimethyl)silyl]oxyethyl]piperidin-3-amine

To a solution of benzyl N-[(3R)-1-[2-[tert-butyl(dimethyl)silyl]oxyethyl]-3-piperidyl]carbamate (2.1 g, 5.35 mmol, 1.0 eq) in methanol (30 mL) was added Pd/C (10%, 200 mg) and Pd(OH)₂ (10%, 200 mg) under N₂. Then the suspension was degassed under vacuum and purged with H₂ several times and stirred under H₂ balloon at 50° C. for 16 h. The suspension was filtered through a pad of Celite and the filter cake was washed with MeOH (3×50 mL), the combined filtrates were concentrated in vacuum to give the desired compound (1100.0 mg, 80% yield) as a yellow oil. ¹H NMR (400 MHz, CD₃OD) δ [ppm]: 3.79 (t, 2H), 2.93-2.91 (m, 1H), 2.83-2.76 (m, 2H), 2.54 (t, 2H), 2.11-2.06 (m, 1H), 1.92-1.84 (m, 2H), 1.73-1.68 (m, 1H), 1.61-1.55 (m, 1H), 1.15-1.06 (m, 1H), 0.90 (s, 9H), 0.83 (s, 6H).

### Intermediate 4

### 3-(Methoxymethyl)-4-(4,4,5,5-tetramethyl-1,3,2-dioxaborolan-2-yl)-5-((2-(trimethylsilyl)ethoxy)methoxy)benzonitrile

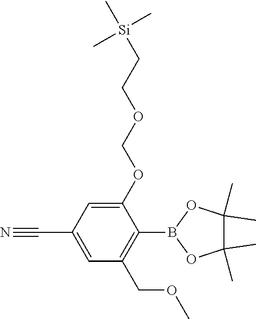 [SMILES: COCc1cc(C#N)cc(OCOCC[Si](C)(C)C)c1B1OC(C)(C)C(C)(C)O1]

### Step A: Methyl 2-amino-5-bromo-3-methoxybenzoate

To a solution of methyl 2-amino-3-methoxy-benzoate (10.0 g, 55.2 mmol, 1.0 eq CAS #5121-34-6) in methanol (40 mL) was added the solution of Br₂ (3.4 mL, 66.2 mmol, 1.2 eq) in acetic acid (20 mL) dropwise at 0-10° C. The reaction mixture was stirred at 20° C. for 2 h. The reaction mixture was poured into aq. NaHSO₃ (20 mL) and extracted with EtOAc (150 mL×3). The organic phase was washed with brine (20 mL), dried over Na₂SO₄, filtered and concentrated under reduced pressure. The residue was purified by column chromatography (silica gel, petroleum ether/ethyl acetate=100/1 to 5/1) to give the title compound (10.0 g, 70% yield) was as a yellow solid. ¹H NMR (400 MHz, DMSO-d₆) δ [ppm]: 7.43 (d, 1H), 7.10 (d, 1H), 6.46 (br. s, 1H), 3.85 (s, 3H), 3.80 (s, 3H).

### Step B: Methyl 2-amino-5-cyano-3-methoxybenzoate

To a solution of methyl 2-amino-5-bromo-3-methoxy-benzoate (10.0 g, 38.45 mmol, 1.0 eq) in NMP (50 mL) was added CuCN (4.13 g, 46.1 mmol, 1.2 eq), The mixture was stirred at 180° C. for 2 h under microwave. The reaction mixture was poured into water (10 mL) and extracted with ethyl acetate (100 ml×3). The organic phase was washed with brine (100 mL×3), dried over Na₂SO₄, filtered and concentrated under reduced pressure. The residue was purified by column chromatography on silica gel (petroleumether:ethyl acetate=1:0 to 5:1) to afford the title compound (6.0 g, 76% yield) as a white solid. LCMS: m/z 207.1 [M+H], ESI pos.

### Step C: Methyl 2-bromo-5-cyano-3-methoxybenzoate

To a solution of methyl 2-amino-5-cyano-3-methoxy-benzoate (5.7 g, 27.64 mmol, 1.0 eq), CuBr (5.95 g, 41.5 mmol, 1.5 eq) in MeCN (80 mL) was added butyl nitrite (4.28 g, 41.5 mmol, 1.5 eq), The mixture was stirred at 65° C. for 2 hours. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography on silica gel (petroleum ether:ethyl acetate=1:0 to 5:1) to afford the title compound (4.0 g, 54% yield) as a white solid. ¹H NMR (400 MHz, DMSO-d₆) δ [ppm]: 7.78 (d, 1H), 7.74 (d, 1H), 3.96 (s, 3H), 3.88 (s, 3H).

### Step D: 2-Bromo-5-cyano-3-hydroxybenzoic acid

To a solution of methyl 2-bromo-5-cyano-3-methoxy-benzoate (3.8 g, 14.1 mmol, 1.0 eq) in DCM (20 mL) was added BBr₃ (13.6 mL, 140.7 mmol, 10.0 eq) at −65° C., then the mixture was stirred at 25° C. for 16 hrs. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography on silica gel (petroleum ether:ethyl acetate=1:0 to 2:1) to afford the title compound (2.9 g, 85% yield) as yellow solid. ¹H NMR (400 MHz, DMSO-d₆) δ [ppm]: 13.76 (br. s, 1H), 11.41 (s, 1H), 7.52 (d, 1H), 7.33 (d, 1H).

### Step E: 2-Trimethylsilylethoxymethyl 2-bromo-5-cyano-3-(2-trimethylsilylethoxymethoxy)-benzoate

A solution of 2-bromo-5-cyano-3-hydroxy-benzoic acid (2.9 g, 11.9 mmol, 1.0 eq), Cs₂CO₃ (7.81 g, 23.96 mmol, 2.0 eq) in DMF (50 mL) was stirred for 10 min at 25° C., then the SEM-Cl (4.0 g, 23.96 mmol, 2.0 eq) was added and stirred at 25° C. for 2 h. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography (silica gel, petroleum ether:ethyl acetate=1:0 to 2:1) to afford the title compound (3.60 g, 60% yield) as a white solid. ¹H NMR (400 MHz, DMSO-d₆) δ [ppm]: 7.83 (d, 1H), 7.78 (d, 1H), 5.49 (d, 4H), 3.82-3.73 (m, 4H), 0.96-0.87 (m, 4H), −0.01 (s, 9H), −0.07 (s, 9H).

### Step F: 4-Bromo-3-(hydroxymethyl)-5-((2-(trimethylsilyl)ethoxy)methoxy)benzonitrile

To a solution of 2-trimethylsilylethoxymethyl 2-bromo-5-cyano-3-(2-trimethylsilylethoxymethoxy)benzoate (3.0 g, 5.97 mmol, 1.0 eq) in THF (20 mL) was added NaBH₄ (903.31 mg, 23.9 mmol, 4.0 eq) and stirred for 16 h at 25° C. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography (silica gel, petroleum ether:ethyl acetate=1:0 to 5:1) to afford the title compound (1.6 g, 75% yield) as a white solid. ¹H NMR (CD₃OD, 400 MHz) δ [ppm]: 7.55 (d, 1H), 7.48 (d, 1H), 5.65 (t, 1H), 5.43 (s, 2H), 4.52 (d, 2H), 3.73 (t, 2H), 0.88 (t, 2H), −0.05 (s, 9H).

### Step G: 4-Bromo-3-(methoxymethyl)-5-((2-(trimethylsilyl)ethoxy)methoxy)benzonitrile

To a solution of 4-bromo-3-(hydroxymethyl)-5-(2-trimethylsilylethoxymethoxy)benzonitrile (200 mg, 0.56 mmol, 1.0 eq) in THF (3 mL) was added NaH (44.7 mg, 1.12 mmol, 2.0 eq 60%) and stirred for 10 mins at 25° C., then Mel (0.1 mL, 1.61 mmol, 2.88 eq) was added and stirred for 2 hours at 25° C. The mixture was quenched with water (30 mL), and extracted with EtOAc (30 mL×3). The organic phase was washed with brine (30 mL×2), dried over anhydrous Na₂SO₄, filtered and concentrated in vacuum. The residue was purified by column chromatography on silica gel (PE/EtOAc, 50:1 to 10:1) to give the title compound (130 mg, 63% yield) was obtained as colorless oil. ¹H NMR (DMSO-d₆, 400 MHz) δ [ppm]: 7.60 (s, 1H), 7.46 (s, 1H), 5.44 (s, 2H), 4.48 (s, 2H), 3.74 (t, 2H), 3.39 (s, 1H), 0.88 (t, 2H), −0.04 (s, 9H).

### Step H: 3-(Methoxymethyl)-4-(4,4,5,5-tetramethyl-1,3,2-dioxaborolan-2-yl)-5-((2-(trimethylsilyl)ethoxy)methoxy)benzonitrile

To a mixture of 4-bromo-3-(methoxymethyl)-5-(2-trimethylsilylethoxymethoxy)benzonitrile (130.0 mg, 0.35 mmol, 1.0 eq), 4,4,4′,4′,5,5,5′,5′-octamethyl-2,2′-bi(1,3,2-dioxaborolane) (441.68 mg, 1.75 mmol, 5.0 eq) in 1,4-dioxane (4 mL) was added KOAc (102.8 mg, 1.05 mmol, 3.0 eq) and Pd(dppf)Cl₂ (25.5 mg, 0.03 mmol, 0.1 eq), then the mixture was stirred at 95° C. for 4 hours. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography on silica gel (PE/EtOAC=1:0 to 10:1) to afford the title compound (20.0 mg, 14% yield) as a white solid. ¹H NMR (CD₃OD, 400 MHz) δ [ppm]: 7.31 (s, 1H), 7.26 (s, 1H), 5.30 (s, 2H), 4.49 (s, 2H), 3.84-3.76 (m, 2H), 3.32 (s, 3H), 1.40 (s, 12H), 0.99-0.92 (m, 2H), 0.01 (s, 9H).

### Intermediate 5

### 5-Bromo-2-chloro-oxazolo[4,5-b]pyridine

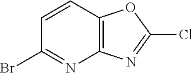 [SMILES: Clc1nc2nc(Br)ccc2o1]

### Step A: 5-Bromooxazolo[4,5-b]pyridine-2-thiol

To a mixture of 2-amino-6-bromo-pyridin-3-ol (CAS #934758-27-7, 5.0 g, 26.45 mmol, 1.0 eq) in DMF (50 mL) was added 1,1′-thiocarbonyldiimidazole (6.13 g, 34.4 mmol, 1.3 eq) and stirred for 16 h at 20° C. Ethyl acetate (100 mL) and water (100 mL) were added and layers were separated. The aqueous phase was extracted with ethyl acetate (2×100 mL). The combined extracts were washed with 1N aq. HCl solution (2×150 mL), flowed by brine (2×200 mL), dried over Na₂SO₄, filtered, and concentrated under vacuum to give the title compound (10.0 g, 96% yield) as a yellow solid. LCMS: m/z 230.9 [M+H]⁺, ESI pos.

### Step B: 5-Bromo-2-chlorooxazolo[4,5-b]pyridine

To a mixture of 5-bromooxazolo[4,5-b]pyridine-2-thiol (9.0 g, 22.86 mmol, 1.0 eq) and DMF (0.1 mL) was added oxalyl chloride (29.02 g, 228.63 mmol, 10.0 eq) and stirred for 1 hour at 50° C. under N₂. The reaction mixture was cooled to room temperature. Ethyl acetate (100 mL) and ice water (50 mL) were added and layers were separated. The aqueous phase was extracted with ethyl acetate (2×100 mL). The combined extracts were washed with brine (2×200 mL) dried over Na₂SO₄, filtered, and concentrated under vacuum. The crude product was purified by column chromatography on silica gel (gradient petroleum ether/ethyl acetate=100:1 to 10:1 to give the title compound (4.0 g, 75% yield) as a yellow solid. LCMS: m/z 234.9 [M+H]⁺, ESI pos.

### Intermediate 6

### Benzyl (3R)-3-[(3-benzyloxy-6-chloro-pyrazin-2-yl)carbamothioylamino]piperidine-1-carboxylate

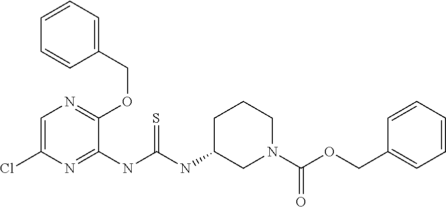 [SMILES: O=C(OCc1ccccc1)N1CCC[C@@H](NC(=S)Nc2nc(Cl)cnc2OCc2ccccc2)C1]

### Step A: Benzyl (3R)-3-isothiocyanatopiperidine-1-carboxylate

To a solution of benzyl (R)-3-aminopiperidine-1-carboxylate (CAS #1044560-96-4, 1.0 g, 4.27 mmol, 1.0 eq) in DCM (5 mL) was a solution of thiophosgene (0.490 g, 4.27 mmol, 1.0 eq) in DCM (5 mL) was added dropwise at 0° C., then stirred at 20° C. for 2 h. The above reaction mixture was purified by column chromatography on silica gel (petroleum ether:ethyl acetate=1:0 to 5:1) to afford the title compound (0.85 g, 72% yield) as yellow oil. ¹H NMR (DMSO-d₆, 400 MHz) δ [ppm]: 7.45-7.27 (m, 5H), 5.12 (s, 2H), 4.06-3.98 (m, 1H), 3.96-3.62 (m, 2H), 3.44-3.36 (m, 1H), 3.28-2.95 (m, 1H), 1.95-1.82 (m, 2H), 1.69-1.45 (m, 2H).

### Step B: Benzyl (3R)-3-[(3-benzyloxy-6-chloro-pyrazin-2-yl)carbamothioylamino]piperidine-1-carboxylate

To a solution of aforementioned 3-benzyloxy-6-chloro-pyrazin-2-amine (Intermediate 2, Step A) (0.600 g, 2.55 mmol, 1.0 eq) in THF (6 mL) was added NaH (203.67 mg, 5.09 mmol, 2.0 eq, 60% in mineral oil) and stirred at 0° C. for 0.5 h, then a solution of benzyl (3R)-3-isothiocyanatopiperidine-1-carboxylate (0.703 g, 2.55 mmol, 1.0 eq) in THF (3 mL) was dropped and stirred at 20° C. for 1 hour. The above reaction solution was quenched with saturated ammonium chloride (100 mL), extracted with ethyl acetate (3×20 mL). The combined organic phase was washed with brine (50 mL), dried over anhydrous sodium sulfate, filtered and the filtrate was concentrated under reduced pressure The residue was purified by column chromatography on silica gel (petroleum ether:ethyl acetate=1:0 to 5:1) to afford the title compound (0.500 g, 36% yield) as yellow gum. LCMS: m/z 512.1, [M+H]⁺, ESI pos.

### Intermediate 7

### (4-Cyano-2-hydroxy-6-methyl-phenyl)boronic Acid

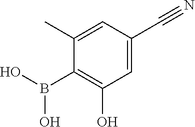

### Step A: 4-Amino-3-methoxy-5-methyl-benzonitrile

Two Batches were carried out in parallel. To a solution of 4-bromo-2-methoxy-6-methyl-aniline (25.0 g, 115 mmol, 1.0 eq) in DMF (250 mL) was added Zn(CN)₂ (13.5 g, 115 mmol, 7.34 mL, 1.00 eq) and Pd(PPh₃)₄ (66.8 g, 57.8 mmol, 0.50 eq). The reaction mixture was stirred at 100° C. for 12 hrs. The reaction mixture was poured into H₂O (1.50 μL) and extracted with ethyl acetate (1.00 μL×3). The organic phase was washed with brine (1.00 μL×3), dried over Na₂SO₄, filtered and concentrated under reduced pressure to give a residue, which was purified by column chromatography (SiO₂, petroleum ether/ethyl acetate=100/1 to 0/1) to yield the title compound (28.0 g, 75% yield) as a yellow solid.

### Step B: 4-Bromo-3-methoxy-5-methyl-benzonitrile

To a solution of CuBr (46.4 g, 323 mmol, 9.86 mL, 1.50 eq) in MeCN (180 mL) was added t-BuONO (33.3 g, 323 mmol, 38.5 mL, 1.50 eq) and stirred at 65° C. Then solution of 4-amino-3-methoxy-5-methyl-benzonitrile (35.0 g, 215 mmol, 1.00 eq) in MeCN (180 mL) at 65° C. was added. The mixture was stirred at 65° C. for 3.5 h. After cooling to rt, sat. aq. Na₂SO₃ (400 mL) and sat. aq. NH₄Cl (200 mL) was added to the mixture and extracted with ethyl acetate (500 mL×3). The organic phase was washed with brine (500 mL×2), dried over Na₂SO₄, filtered and concentrated under reduced pressure to give a residue. The residue was purified by column chromatography (SiO₂, petroleum ether/ethyl acetate=100/1 to 0/1, R_{f}=0.75) to give the title compound (20.7 g, 42% yield) as a white solid.

### Step C: 3-Methoxy-5-methyl-4-(4,4,5,5-tetramethyl-1,3,2-dioxaborolan-2-yl)benzonitrile

To a solution of 4-bromo-3-methoxy-5-methyl-benzonitrile (18.0 g, 79.6 mmol, 1.00 eq) in DMF (180 mL) was added B₂Pin₂ (30.3 g, 119 mmol, 1.50 eq) and AcOK (35.1 g, 358 mmol, 4.50 eq). The mixture was stirred at 20° C. for 0.5 hr and Pd(dppf)Cl₂·CH₂Cl₂ (13.0 g, 15.9 mmol, 0.20 eq) was added. The mixture was stirred at 100° C. for 12 h. After cooling to rt, the mixture was filtered with diatomite and diluted with H₂O (500 mL) and extracted with ethyl acetate (800 mL×3). The organic phase was washed with brine (800 mL×3), dried over Na₂SO₄, filtered and concentrated under reduced pressure. The residue was purified by column chromatography (SiO₂, petroleum ether/ethyl acetate=100/1 to 1/1, R_{f}=0.30) to give the title compound (18.0 g, 83% yield) as a white solid.

### Step D: (4-Cyano-2-hydroxy-6-methyl-phenyl)boronic Acid

A solution of 3-methoxy-5-methyl-4-(4,4,5,5-tetramethyl-1,3,2-dioxaborolan-2-yl)benzonitrile (17.0 g, 96.0 mmol, 1.00 eq) in DCM (170 mL) was cooled to 0° C. and BBr₃ (38.9 g, 155 mmol, 2.50 eq) was added dropwise at 0° C. The mixture was stirred at 0° C. for 0.5 h. The mixture was poured into H₂O (200 mL), filtered, and the cake was collected and triturated with ethyl acetate (20 mL) to give the title compound (4.67 g, 42% yield) as a gray solid. LCMS: m/z 178.1 [M+H]⁺, ESI pos.

### Intermediate 8

### 6-(Methoxymethyl)-5-(4,4,5,5-tetramethyl-1,3,2-dioxaborolan-2-yl)-2,3-dihydrobenzofuran-4-ol

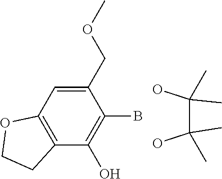

### Step A: 2-[2,6-Dibromo-4-(methoxymethyl)phenyl]ethanol

To a solution of 1,3-dibromo-5-(methoxymethyl)benzene (171 g, 610 mmol, 1.00 eq, CAS #1646314-10-4) in THF (750 mL) was added LDA (2.00 M, 366 mL, 1.20 eq) at −78° C. and stirring was continued for 30 mins. Afterwards, a solution of 1,3,2-dioxathiolane 2,2-dioxide (98.5 g, 794 mmol, 1.30 eq, CAS #1072-53-3) in THF (750 mL) was added to the mixture at −78° C. The mixture was warmed and stirred at 25° C. for 1 h. The reaction mixture was quenched by addition of aq. HCl (12.0 M, 170 mL) at 0° C., and then diluted with water (500 mL) and extracted with EtOAc (500 mL×3). The combined organic layers were washed with brine (500 mL), dried over Na₂SO₄, filtered and concentrated under reduced pressure. The crude was purified by column chromatography on silica gel (petroleum ether/ethyl acetate=50/1 to 10/1) to yield the title compound (60.0 g, 30% yield) as a white solid NLRP.

### Step B: 4-Bromo-6-(methoxymethyl)-2,3-dihydrobenzofuran

To a solution of 2-[2,6-dibromo-4-(methoxymethyl)phenyl]ethanol (60.0 g, 185 mmol, 1.00 eq) in dioxane (600 mL) was added t-BuOLi (44.4 g, 555 mmol, 50.0 mL, 3.00 eq) and CuI (3.53 g, 18.5 mmol, 0.10 eq) under N₂ atmosphere. The mixture was stirred at 100° C. for 5 hours. The reaction mixture was quenched by addition water (1.00 μL), and then diluted with ethyl acetate (500 mL) and extracted with ethyl acetate (500 mL×3). The combined organic layers were washed with brine (500 mL), dried over Na₂SO₄, filtered and concentrated under reduced pressure. The title compound (50.0 g, crude) was obtained as a yellow oil and used without further purification in the next step.

### Step C: 6-(Methoxymethyl)-2,3-dihydrobenzofuran-4-ol

To a solution of 4-bromo-6-(methoxymethyl)-2,3-dihydrobenzofuran (48.0 g, 197 mmol, 1.00 eq) in dioxane (250 mL) and H₂O (250 mL) was added t-BuXphos (16.7 g, 39.4 mmol, 0.20 eq), Pd₂ (dba)₃ (9.04 g, 9.87 mmol, 0.05 eq) and KOH (33.2 g, 592 mmol, 3.00 eq). The mixture was stirred at 80° C. for 16 h under N₂ atmosphere. The mixture was adjusted to pH=5 with aq. HCl (12 M), and then extracted with ethyl acetate (500 mL×3). The combined organic layers were washed with brine (500 mL), dried over Na₂SO₄, filtered and concentrated under reduced pressure. The crude was purified by column chromatography on silica gel (petroleum ether/ethyl acetate=50/1 to 5/1) to afford the title compound (26 g, 74% yield) as a white solid.

### Step D: 5-Bromo-6-(methoxymethyl)-2,3-dihydrobenzofuran-4-ol

To a solution of 6-(methoxymethyl)-2,3-dihydrobenzofuran-4-ol (25.0 g, 138 mmol, 1.00 eq) in DCM (250 mL) was added NBS (22.2 g, 124 mmol, 0.90 eq) at 0° C. The mixture was stirred at 0° C. for 5 hours. The reaction mixture was concentrated under reduced pressure and the residue was purified by column chromatography on silica gel (petroleum ether/ethyl acetate: 50/1 to 10/1) to yield the title compound (16.0 g, 45% yield) as a white solid. ¹H NMR (400 MHz, CDCl₃) δ [ppm]: 6.56 (s, 1H), 5.72 (s, 1H), 4.63 (t, 2H), 4.44 (s, 2H), 3.43 (s, 3H), 3.22 (t, 2H).

### Step E: 4-Benzyloxy-5-bromo-6-(methoxymethyl)-2,3-dihydrobenzofuran

To a solution of 5-bromo-6-(methoxymethyl)-2,3-dihydrobenzofuran-4-ol (15.0 g, 57.8 mmol, 1.00 eq) in MeCN (150 mL) was added BnBr (10.8 g, 63.6 mmol, 7.56 mL, 1.10 eq) and K₂CO₃ (16.0 g, 115 mmol, 2.00 eq). The mixture was stirred at 25° C. for 2 hours. The reaction mixture was concentrated under reduced pressure to give a residue. The residue was diluted with water (100 mL) and extracted with EtOAc (50 mL×3). The combined organic layers were washed with brine (50 mL), dried over Na₂SO₄, filtered and concentrated under reduced pressure to give a residue. The crude product was triturated with petroleum ether (50.0 mL) at 25° C. for 16 h. The title compound (10.0 g, 74% yield) was obtained as a white solid. LCMS: m/z 350.0 [M+H]⁺, ESI pos.

### Step F: 2-[4-Benzyloxy-6-(methoxymethyl)-2,3-dihydrobenzofuran-5-yl]-4,4,5,5-tetramethyl-1,3,2-dioxaborolane

To a solution of 4-benzyloxy-5-bromo-6-(methoxymethyl)-2,3-dihydrobenzofuran (1.5 g, 4.08 mmol, 1.00 eq) and 2-isopropoxy-4,4,5,5-tetramethyl-1,3,2-dioxaborolane (1.19 g, 1.3 mL, 6.37 mmol, 1.56 eq) in tetrahydrofuran (15 mL) was added dropwise 1.6 M n-butyllithium, solution in hexane (4 mL, 6.4 mmol, 1.57 eq) at −76° C. and stirring was continued at −76° C. for 2.25 h. The reaction mixture was warmed to −60° C., quenched with saturated aq. NH₄Cl-solution (˜10 mL) at −60° C., warmed to room temperature and then extracted with ethyl acetate (˜70 mL) and saturated aq. NH₄Cl-solution (˜10 mL). The aqueous layer was backextracted with ethyl acetate (˜70 mL). The organic layers were washed with brine (˜10 mL). The combined organic layers were dried over sodium sulfate, filtered and concentrated in vacuo. The crude product was purified by flash chromatography (silica gel, 25 g, gradient 0% to 20% ethyl acetate in heptane) to afford the title compound (1.26 g, 74% yield) as colorless oil. LCMS: m/z 397.3 [M+H]⁺, ESI pos.

### Step F: 6-(Methoxymethyl)-5-(4,4,5,5-tetramethyl-1,3,2-dioxaborolan-2-yl)-2,3-dihydrobenzofuran-4-ol

A solution of 2-[4-benzyloxy-6-(methoxymethyl)-2,3-dihydrobenzofuran-5-yl]-4,4,5,5-tetramethyl-1,3,2-dioxaborolane (1.24 g, 2.98 mmol, 1.00 eq) in ethyl acetate (15 mL) and methanol (2.5 mL) was three times alternating evacuated and flushed with argon. Palladium on activated charcoal, 10% Pd basis (136 mg, 127.8 μmol, 0.043 eq) was added carefully. The reaction flask was evacuated, flushed with argon, evacuated and flushed with hydrogen. The reaction mixture was stirred under hydrogen atmosphere (balloon) at room temperature for 3 h. Afterwards, the reaction mixture was filtered and rinsed with ethyl acetate/methanol. The filtrate was concentrated in vacuo to afford the title compound (929 mg, 97% yield) as light brown solid. LCMS: m/z 307.2 [M+H]⁺, ESI pos. The compound was used without further purification.

### Intermediate 9

### (3S,5R)-5-[(5-Bromooxazolo[4,5-b]pyrazin-2-yl)amino]-1-ethyl-piperidin-3-ol

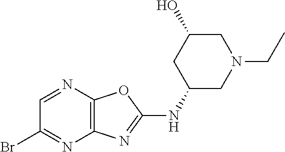 [SMILES: CCN1C[C@@H](O)C[C@@H](Nc2nc3nc(Br)cnc3o2)C1]

### Step A: 2-Benzyloxy-5-bromo-3-isothiocyanato-pyrazine

To a solution of 3-benzyloxy-6-bromo-pyrazin-2-amine (2.0 g, 7.14 mmol, 1.0 eq; CAS #2767116-11-8) in DCM (20 mL) was added 1,1′-thiocarbonyldipyridin-2(1H)-one (1.99 g, 8.57 mmol, 1.2 eq). The reaction mixture was stirred at 30° C. for 16 hours. Upon the reaction completion, the reaction mixture was concentrated in vacuum. The residue was purified by silica gel column chromatography (petroleum ether:ethyl acetate=100:1 to 50:1) to give the title compound (1.2 g, 51% yield) as a yellow solid. ¹H NMR (400 MHz, DMSO-d₆) δ [ppm]: 8.39 (s, 1H), 7.52-7.50 (m, 2H), 7.43-7.35 (m, 3H), 5.47 (s, 2H).

### Step B: tert-butyl (3R,5S)-3-[(3-Benzyloxy-6-bromo-pyrazin-2-yl)carbamothioylamino]-5-hydroxy-piperidine-1-carboxylate

To a mixture of tert-butyl (3R,5S)-3-amino-5-hydroxy-piperidine-1-carboxylate (671.3 mg, 3.1 mmol, 1.0 eq) and DIEA (0.81 mL, 4.66 mmol, 1.5 eq) in DCM (10 mL) was added dropwise a solution of 2-benzyloxy-5-bromo-3-isothiocyanato-pyrazine (1000.0 mg, 3.1 mmol, 1.0 eq) in DCM (10 mL). The reaction mixture was stirred at 20° C. for 1 hour under N₂. Upon the reaction completion, the reaction mixture was quenched with water (30 mL), and extracted with ethyl acetate (50 mL×2). The organic phases were washed with brine (50 mL×2), dried over anhydrous Na₂SO₄, filtered and the filtrate was concentrated in vacuum. The residue was purified by silica gel column chromatography (petroleum ether:ethyl acetate=59%˜41%) to yield the title compound (1580.0 mg, 95% yield) as yellow oil. LCMS: m/z 484.1 [M-C4H8+H]⁺, ESI pos. ¹H NMR (400 MHz, DMSO-d₆) δ [ppm]: 10.47 (d, 1H), 8.86 (s, 1H), 7.99 (s, 1H), 7.51-7.49 (m, 2H), 7.43-7.34 (m, 3H), 5.48-5.40 (m, 2H), 5.08 (br s, 1H), 4.36-4.26 (m, 1H), 3.71-3.67 (m, 1H), 3.56-3.54 (m, 1H), 3.44-3.35 (m, 1H), 3.23-3.22 (m, 1H), 3.29-3.22 (m, 1H), 2.15-2.05 (m, 1H), 1.68-1.58 (m, 1H), 1.25 (br s, 9H).

### Step C: 1-(6-Bromo-3-hydroxy-pyrazin-2-yl)-3-[(3R,5S)-5-hydroxy-3-piperidyl]thiourea

To a solution of tert-butyl (3R,5S)-3-[(3-benzyloxy-6-bromo-pyrazin-2-yl)carbamothioylamino]-5-hydroxy-piperidine-1-carboxylate (1800.0 mg, 3.34 mmol, 1.0 eq) in anisole (3 mL) was added TFA (3 mL) and the mixture was stirred at 70° C. for 2 hours under N₂. Upon the reaction completion, the mixture was concentrated in vacuum. The residue was purified by prep-HPLC (column: Xtimate C18, 250 mm*50 mm*10 μm; mobile phase: [water (0.1% ammonia hydroxide v/v)-MeCN]; B %: 5%-39%, 15 minutes), the eluent was lyophilized to give the title compound (750.0 mg, 65% yield) as a yellow solid. LCMS: m/z 350.0[M+2+H]⁺, ESI pos.

### Step D: (3S,5R)-5-[(5-Bromooxazolo[4,5-b]pyrazin-2-yl)amino]piperidin-3-ol

To a solution of 1-(6-bromo-3-hydroxy-pyrazin-2-yl)-3-[(3R,5S)-5-hydroxy-3-piperidyl]thiourea (50.0 mg, 0.14 mmol, 1.0 eq), tetrabutylammonium iodide (10.6 mg, 0.03 mmol, 0.2 eq) in THF (2 mL) and methanol (0.2 mL) was added H₂O₂ (240.0 mg, 2.12 mmol, 14.74 eq, purity: 30% in water). The resulting mixture was stirred at 20° C. for 1 hour. Upon the reaction completion, the mixture was quenched with saturated Na₂SO₃ aqueous solution (0.2 mL), and concentrated in vacuum. The residue was purified by prep-HPLC (column: Phenomenex Gemini, 150 mm*30 mm*15 um; mobile phase: [water (0.1% TFA, V/V)-MeCN]; B %: 22%-40%, 10 mins), the eluent was lyophilized to give the title compound (10.0 mg, 22% yield) as a white solid. LCMS: m/z 314.1 [M+H]⁺, ESI pos.

### Step E: (3S,5R)-5-[(5-Bromooxazolo[4,5-b]pyrazin-2-yl)amino]-1-ethyl-piperidin-3-ol

To a solution of (3S,5R)-5-[(5-bromooxazolo[4,5-b]pyrazin-2-yl)amino]piperidin-3-ol (110.0 mg, 0.26 mmol, 1.0 eq), DIEA (0.09 mL, 0.51 mmol, 2.0 eq) in DMF (1 mL) was added CH₃CH₂I (0.02 mL, 0.26 mmol, 1.0 eq). The reaction mixture was stirred at 20° C. for 1 hour under nitrogen. Upon the reaction completion, the mixture was purified by prep-HPLC (column: Xtimate C18, 250 mm*50 mm*10 μm; mobile phase: [water (0.1% ammonia hydroxide v/v)-MeCN]; B %: 10%-45%, 8 minutes), the eluent was lyophilized to give the title compound (40.0 mg, 26% yield) as a yellow solid. LCMS: m/s 343.9[M+H]⁺, ESI pos.

### Intermediate 10

### 2,5-Dichloro-7-fluoro-oxazolo[4,5-b]pyridine

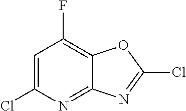 [SMILES: Fc1cc(Cl)nc2nc(Cl)oc12]

### Step B: 6-Chloro-4-fluoro-2-nitro-pyridin-3-ol

To a solution of 6-chloro-4-fluoro-pyridin-3-ol (4.3 g, 29.15 mmol, 1.0 eq; CAS #1211530-63-0) in sulfuric acid (40.0 mL, 116.59 mmol, 4.0 eq.) was slowly added HNO₃ (7.06 g, 100.86 mmol, 3.46 eq.) at 0° C., then the mixture was stirred at 20° C. for 4 hours. Upon the reaction was completion, the above reaction solution was diluted with water (200 mL), and extracted with ethyl acetate (100 mL×3). The combined organic phases were washed with brine (200 mL), dried over anhydrous sodium sulfate, filtered and the filtrate was concentrated under reduced pressure. The residue was purified by column chromatography (SiO₂, petroleum ether:ethyl acetate=1:1 to 10:1) to afford the title compound (5.0 g) as a yellow solid. ¹H NMR (400 MHz, DMSO-d₆) ([ppm]: 8.03 (d, 1H).

### Step C: 2-Amino-6-chloro-4-fluoro-pyridin-3-ol

To a solution of 6-chloro-4-fluoro-2-nitro-pyridin-3-ol (2.1 g, 10.91 mmol, 1.0 eq.) in ethanol (30 mL) and water (30 mL) was added Na₂S204 (11.39 g, 65.44 mmol, 6.0 eq.), then stirred at 70° C. for 3 hours. Upon the reaction was completion, the above reaction solution was cooled to room temperature, diluted with water (100 mL), and extracted with ethyl acetate (50 mL×3). The combined organic phases were washed with brine (100 mL), dried over anhydrous sodium sulfate, filtered and the filtrate was concentrated under reduced pressure. The residue was purified by column chromatography (SiO₂, petroleum ether:ethyl acetate=1:1 to 0:1) to afford the title compound (1.27 g, 71% yield) as a yellow solid. LCMS: m/z 163.1, [M+H]⁺, ESI pos.

### Step D: 5-Chloro-7-fluoro-oxazolo[4,5-b]pyridine-2-thiol

To a solution of 2-amino-6-chloro-4-fluoro-pyridin-3-ol (500.0 mg, 3.08 mmol, 1.0 eq.) in DMF (5 mL) was added 1,1′-thiocarbonyldiimidazole (603.0 mg, 3.38 mmol, 1.1 eq.), then the mixture was stirred at 50° C. for 2 hours. Upon the reaction was completion, the above reaction solution was cooled to room temperature, diluted with water (20 mL), and extracted with ethyl acetate (20 mL×3). The combined organic phases were washed with 1M HCl (50 mL), brine (50 mL), dried over anhydrous sodium sulfate, filtered and the filtrate was concentrated under reduced pressure. The residue was purified by column chromatography (SiO₂, petroleum ether:ethyl acetate=1:0 to 1:1) to afford the title compound (320.0 mg, 47% yield) as a white solid. LCMS: m/z 205.0, [M+H]⁺, ESI⁺ pos.

### Step E: 2,5-Dichloro-7-fluoro-oxazolo[4,5-b]pyridine

To a solution of 5-chloro-7-fluoro-oxazolo[4,5-b]pyridine-2-thiol (320.0 mg, 1.56 mmol, 1.0 eq.) in oxalyl dichloride (4.0 mL, 46.96 mmol, 30.0 eq.) was added DMF (11.4 mg, 0.16 mmol, 0.1 eq.). And then the mixture was stirred at 50° C. for 1 hour. Upon the reaction was completion, the above reaction solution was cooled to room temperature, diluted with water (20 mL), and extracted with ethyl acetate (10 mL×3). The combined organic phases were dried over anhydrous sodium sulfate, filtered and the filtrate was concentrated under reduced pressure. The residue was purified by column chromatography (SiO₂, petroleum ether:ethyl acetate=1:0 to 10:1) to afford the title compound (300.0 mg, 93% yield) as a yellow solid.

### Intermediate 11

### 1-(6-Bromo-3-hydroxy-pyrazin-2-yl)-3-[(3R)-1-(2-hydroxyethyl)-3-piperidyl]thiourea

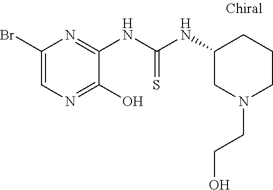 [SMILES: OCCN1CCC[C@@H](NC(=S)Nc2nc(Br)cnc2O)C1]

### Step A: 1-(3-Benzyloxy-6-bromo-pyrazin-2-yl)-3-[(3R)-1-(2-hydroxyethyl)-3-piperidyl]-thiourea

To a solution of 1-(6-bromo-3-hydroxy-pyrazin-2-yl)-3-[(3R)-3-piperidyl]thiourea; hydrochloride (2600.0 mg, 7.03 mmol, 1.0 eq) in DCE (30 mL) and methanol (3 mL) was added CH₃COONa (1730.73 mg, 21.1 mmol, 3.0 eq) followed by CH₃COOH (42.2 mg, 0.7 mmol, 0.1 eq) to adjust pH to 5.0 and (tert-butyldimethylsilyloxy)acetaldehyde (1838.97 mg, 10.55 mmol, 1.5 eq) and the mixture was stirred at 10 mins before adding NaBH(OAc)₃ (4471.67 mg, 21.1 mmol, 3.0 eq). Stirring at 20° C. was continued for 1 hour under N2. Then, the mixture was quenched with water (30 mL), and extracted with EtOAc (60 mL×2), the organic phase was washed with brine (60 mL×2), dried over anhydrous Na₂S04, filtered and concentrated in vacuum. The residue was purified by C18 column chromatography (0.10% TFA-CAN=54%˜46% to 36% 64%) to give 1-(6-bromo-3-hydroxy-pyrazin-2-yl)-3-[(3R)-1-[2-[tert-butyl(dimethyl)silyl]oxyethyl]-3-piperidyl]thiourea (100.0 mg, 3% yield) as a yellow solid (LCMS m/z: 492.2 [M+2+H]⁺, ESIpos) and the title compound (1200.0 mg, 24% yield) as a yellow solid. LCMS m/z: 466.2 [M+H]⁺, ESI pos.

### Step B: 1-(6-Bromo-3-hydroxy-pyrazin-2-yl)-3-[(3R)-1-(2-hydroxyethyl)-3-piperidyl]thiourea

To a solution of 1-(3-benzyloxy-6-bromo-pyrazin-2-yl)-3-[(3R)-1-(2-hydroxyethyl)-3-piperidyl]-thiourea (1200.0 mg, 2.57 mmol, 1.0 eq) in TFA (2 mL) stirred at 70° C. for 2 hours under N2. Upon reaction completion, the mixture was concentrated in vacuum to give a residue, which was then purified by C18 column chromatography (0.10% NH₃·H₂O—CAN=54%˜46%) to give the title compound (300.0 mg, 21% yield) as a white solid. LCMS m/z: 376.1 [M+H]⁺, ESI pos.

### PREPARATION OF EXAMPLES

### Example 1

### 3-Hydroxy-4-[2-[[(3R)-1-(2-hydroxyethyl)-3-piperidyl]amino]oxazolo[4,5-b]pyrazin-5-yl]-5-methyl-benzonitrile;2,2,2-trifluoroacetic Acid

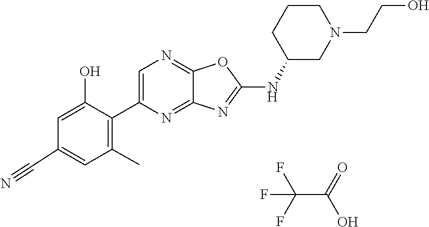

### Step A: 1-(6-Chloro-3-hydroxy-pyrazin-2-yl)-3-[(3R)-3-piperidyl]thiourea

To a solution of benzyl (3R)-3-[(3-benzyloxy-6-chloro-pyrazin-2-yl)carbamothioylamino]piperidine-1-carboxylate (1.5 g, 2.93 mmol, 1.0 eq) in TFA (15.0 mL, 197.37 mmol, 67.37 eq) was added anisole (7.5 mL, 69.01 mmol, 23.56 eq) and the reaction mixture was stirred at 75° C. for 12 h. Afterwards, the mixture was concentrated under reduced pressure and purified by reversed phase flash (CombiFlash NH₃—H₂O water-MeCN condition) to give the title compound (320.0 mg, 22% yield) as a yellow solid. LCMS: m/z 288.2 [M+H]⁺, ESI pos.

### Step B: 1-[(3R)-1-[2-[tert-Butyl(dimethyl)silyl]oxyethyl]-3-piperidyl]-3-(6-chloro-3-hydroxy-pyrazin-2-yl)thiourea

To a solution of 1-(6-chloro-3-hydroxy-pyrazin-2-yl)-3-[(3R)-3-piperidyl]thiourea (270.0 mg, 0.94 mmol, 1.0 eq) in methanol (5 mL)/DCE (10 mL) was added (tert-butyldimethylsilyloxy)acetaldehyde (CAS #102191-92-4, 327.1 mg, 1.88 mmol, 2.0 eq) followed by NaBH(OAc)₃ (994.3 mg, 4.69 mmol, 5.0 eq) and stirring was continued at 20° C. for 1.5 h. The above reaction mixture was poured into ice (20 mL) and extracted with ethyl acetate (3×50 mL). The combined organic phase was washed with brine (3×50 mL), dried over anhydrous sodium sulfate, filtered and the filtrate was concentrated under reduced pressure and purified over column chromatography on silica gel (hexane/EtOAc, 1:0 to 0:1) to give the title compound (220.0 mg, 49% yield) as a yellow solid. LCMS: m/z 446.1 [M+H]⁺, ESI pos.

### Step C: N-[(3R)-1-[2-[tert-Butyl(dimethyl)silyl]oxyethyl]-3-piperidyl]-5-chloro-oxazolo[4,5-b]pyrazin-2-amine

To a mixture of 1-[(3R)-1-[2-[tert-butyl(dimethyl)silyl]oxyethyl]-3-piperidyl]-3-(6-chloro-3-hydroxy-pyrazin-2-yl)thiourea (220.0 mg, 0.49 mmol, 1.0 eq) and tetrabutylazanium; iodide (200.4 mg, 0.54 mmol, 1.1 eq) in THF (10 mL) was added H₂O₂ (111.8 mg, 0.99 mmol, 2.0 eq) and the mixture was stirred at 25° C. for 2 h. The residue was purified by silica gel column chromatography (EtOAc/MeOH=1:0 to 10:1) to afford the title compound (80.0 mg, 40% yield) as a yellow solid.

LCMS: m/z 412.1 [M+H]⁺, ESI pos.

### Step D: 4-[2-[[(3R)-1-[2-[tert-Butyl(dimethyl)silyl]oxyethyl]-3-piperidyl]amino]oxazolo[4,5-b]pyrazin-5-yl]-3-methyl-5-(2-trimethylsilylethoxymethoxy)benzonitrile

To a mixture of 3-methyl-4-(4,4,5,5-tetramethyl-1,3,2-dioxaborolan-2-yl)-5-(2-trimethylsilylethoxymethoxy)benzonitrile Intermediate 1 (79.4 mg, 0.2 mmol, 1.2 eq), N-[(3R)-1-[2-[tert-butyl(dimethyl)silyl]oxyethyl]-3-piperidyl]-5-chloro-oxazolo[4,5-b]pyrazin-2-amine (step C) (70.0 mg, 0.17 mmol, 1.0 eq), CsF (77.42 mg, 0.51 mmol, 3.0 eq) in 1,4-dioxane (4 mL)/water (0.5 mL) was added Pd-PEPPSI-iHeptCl (CAS #905459-27-0, 14.4 mg, 0.02 mmol, 0.1 eq) under N₂ and stirred for 3 h at 95° C. The above reaction mixture was cooled to room temperature, poured into ice (10 mL) and extracted with ethyl acetate (3×50 mL). The combined organic phase was washed with brine (3×50 mL), dried over anhydrous sodium sulfate, filtered and the filtrate was concentrated under reduced pressure, The crude product was purified by reversed phase flash (CombiFlash 0.1% TFA water-MeCN condition) to give the title compound (30.0 mg, 26% yield) as a yellow solid. LCMS: m/z 639.4 [M+H]⁺, ESI pos.

### Step E: 3-Hydroxy-4-[2-[[(3R)-1-(2-hydroxyethyl)-3-piperidyl]amino]oxazolo[4,5-b]pyrazin-5-yl]-5-methyl-benzonitrile;2,2,2-trifluoroacetic Acid

To a mixture of 4-[2-[[(3R)-1-[2-[tert-butyl(dimethyl)silyl]oxyethyl]-3-piperidyl]amino]-oxazolo[4,5-b]pyrazin-5-yl]-3-methyl-5-(2-trimethylsilylethoxymethoxy)benzonitrile (step D) (25.0 mg, 0.04 mmol, 1.0 eq) in DCM (0.1 mL) was added TFA (0.1 mL) and stirred for 1 h at 25° C. The above reaction mixture was concentrated under reduced pressure. The crude product was purified by reversed phase flash (CombiFlash 0.1% TFA water-MeCN condition) to afford the title compound (14.3 mg, 71% yield) as a yellow solid. LCMS: m/z 395.1 [M+H]⁺, ESI pos.

### Example 2

### 4-[2-[[(3R,5S)-1-Ethyl-5-hydroxy-3-piperidyl]amino]oxazolo[4,5-b]pyrazin-5-yl]-3-hydroxy-5-methyl-benzonitrile;2,2,2-trifluoroacetic Acid

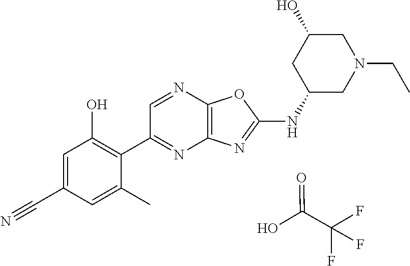 [SMILES: CCN1C[C@@H](O)C[C@@H](Nc2nc3nc(-c4c(C)cc(C#N)cc4O)cnc3o2)C1 ; O=C(O)C(F)(F)F]

### Step A: tert-Butyl (3R,5S)-3-amino-5-[tert-butyl(diphenyl)silyl]oxy-piperidine-1-carboxylate

To a solution of tert-butyl (3R,5S)-3-amino-5-hydroxy-piperidine-1-carboxylate (CAS #1932513-59-1, 400 mg, 1.85 mmol, 1.0 eq) and 1H-imidazole (378 mg, 5.55 mmol, 3.0 eq) in DMF (2 mL) was added tert-butylchlorodiphenylsilane (1029 mg, 3.74 mmol, 2.0 eq). Then the mixture was stirred at 20° C. for 48 h. Upon reaction completion ethyl acetate (40 mL) and water (20 mL) were added and layers were separated. The aqueous phase was extracted with ethyl acetate (40 mL×2). The combined extracts were washed with brine (50 mL), dried over Na₂SO₄, filtered, and concentrated under vacuum. The residue was purified by column chromatography on silica gel (petroleum ether:ethyl acetate=1:2 to 0:1) to give the title compound (230.0 mg, 27% yield) as yellow oil. LCMS: m/z 455.4 [M+H]⁺, ESI pos.

### Step B: tert-Butyl (3S,5R)-3-[tert-butyl(diphenyl)silyl]oxy-5-isothiocyanato-piperidine-1-carboxylate

To a stirring solution of TEA (102 mg, 1.01 mmol, 2.0 eq) and tert-butyl (3R,5S)-3-amino-5-[tert-butyl(diphenyl)silyl]oxy-piperidine-1-carboxylate (230.0 mg, 0.51 mmol, 1.0 eq) in DCM (2 mL) was added a solution of thiophosgene (174.5 mg, 1.52 mmol, 3.0 eq) at 0° C. dropwise and stirring was continued for 2 h at 25° C. Upon reaction completion, the residue was purified by column chromatography on silica gel (petroleum ether:ethyl acetate=5:1 to 4:1) to give the title compound (130.0 mg, 52% yield) as yellow oil. LCMS: m/z 519.3 [M+Na]⁺, ESI pos.

### Step C: tert-Butyl (3R,5S)-3-[[3-benzyloxy-6-[4-cyano-2-methyl-6-(2-trimethylsilylethoxy-methoxy)phenyl]pyrazin-2-yl]carbamothioylamino]-5-[tert-butyl(diphenyl)silyl]oxy-piperidine-1-carboxylate

To a solution of 4-(6-amino-5-benzyloxy-pyrazin-2-yl)-3-methyl-5-(2-trimethylsilylethoxymethoxy)benzonitrile Intermediate 2 (100.0 mg, 0.22 mmol, 1.0 eq) in THF (7 mL) was added NaH (60% in mineral oil, 17.3 mg, 0.43 mmol, 2.0 eq) and stirred at 20° C. for 0.5 h. Afterwards, a solution of tert-butyl (3S,5R)-3-[tert-butyl(diphenyl)silyl]oxy-5-isothiocyanato-piperidine-1-carboxylate (108.0 mg, 0.22 mmol, 1.01 eq) in THF (2 mL) was added dropwise at 0° C. and the reaction was stirred at 20° C. for 4 h. As the reaction mixture was insoluble additional DMF (10.0 mL) was added and stirring was continued at 20° C. for 1 h. Upon the reaction completion, the reaction mixture was cooled to room temperature. Ethyl acetate (50 mL) and saturated aq. CaCl₂) solution (50 mL) were added and layers were separated. The aqueous phase was extracted with ethyl acetate (30 mL×2). The combined extracts were washed with saturated aq. CaCl₂ solution (50 mL), dried over Na₂SO₄, filtered, and concentrated under vacuum. The residue was purified by column chromatography on silica gel (petroleum ether:ethyl acetate=1:1 to 1:2) to give the title compound (100.0 mg, 48% yield) as yellow oil. LCMS: m/z 959.2 [M+H]⁺, ESI pos.

### Step D: 1-[(3R,5S)-5-[tert-Butyl(diphenyl)silyl]oxy-3-piperidyl]-3-[6-(4-cyano-2-hydroxy-6-methyl-phenyl)-3-hydroxy-pyrazin-2-yl]thiourea

tert-Butyl (3R,5S)-3-[[3-benzyloxy-6-[4-cyano-2-methyl-6-(2-trimethylsilylethoxy-methoxy)-phenyl]pyrazin-2-yl]carbamothioylamino]-5-[tert-butyl-(diphenyl)silyl]oxy-piperidine-1-carboxylate (100.0 mg, 0.1 mmol, 1.0 eq) was dissolved in TFA (3.0 mL, 39.5 mmol, 378.7 eq) and stirred at 20° C. for 2 h. Then the mixture was concentrated in vacuum and the residue was dissolved with methanol (2 mL) and the pH was adjusted to 7 with NH₃·H₂O. Afterwards, the crude was purified by reversed phase flash (CombiFlash 0.1% NH₃·H₂O aqueous-ACN condition) to afford the title compound (40.0 mg, 60% yield) as a white solid. LCMS: m/z 639.3 [M+H]⁺, ESI pos.

### Step E: 1-[(3R,5S)-5-[tert-Butyl(diphenyl)silyl]oxy-1-ethyl-3-piperidyl]-3-[6-(4-cyano-2-hydroxy-6-methyl-phenyl)-3-hydroxy-pyrazin-2-yl]thiourea

To a solution of 1-[(3R,5S)-5-[tert-butyl(diphenyl)silyl]oxy-3-piperidyl]-3-[6-(4-cyano-2-hydroxy-6-methyl-phenyl)-3-hydroxy-pyrazin-2-yl]thiourea (40 mg, 0.06 mmol, 1.0 eq) in methanol (0.1 mL) and DCE (1 mL) was added acetaldehyde (0.04 mL, 0.19 mmol, 3.0 eq). The mixture was stirred for 15 min, then NaBH(OAc)₃ (53 mg, 0.25 mmol, 4.0 eq) was added and stirred at 20° C. for 1.5 h. The mixture was purified by reversed phase flash (CombiFlash 0.1% NH₃·H₂O aqueous-MeCN condition) to afford the title compound (25.0 mg, 60% yield) as a white solid. LCMS: m/z 667.3 [M+H]⁺, ESI pos.

### Step F: 4-[2-[[(3R,5S)-5-[tert-Butyl(diphenyl)silyl]oxy-1-ethyl-3-piperidyl]amino]oxazolo-[4,5-b]pyrazin-5-yl]-3-hydroxy-benzonitrile; 2,2,2-trifluoroacetic Acid

To a solution of 1-[(3R,5S)-5-[tert-butyl(diphenyl)silyl]oxy-1-ethyl-3-piperidyl]-3-[6-(4-cyano-2-hydroxy-6-methyl-phenyl)-3-hydroxy-pyrazin-2-yl]thiourea (30.0 mg, 0.04 mmol, 1.0 eq) and tetrabutylazanium iodide (16.6 mg, 0.04 mmol, 1.0 eq) in THF (1 mL) was added H₂O₂ (70.0 mg, 0.62 mmol, 13.7 eq) and stirring was continued at 25° C. for 1 h. The mixture was then concentrated under vacuum. Afterwards, the residue was purified by reversed phase flash (CombiFlash 0.1% TFA aqueous-MeCN condition) to afford the title compound (20.0 mg, 60% yield) as yellow solid. LCMS: m/z 633.2 [M+H]⁺, ESI pos.

### Step G: 4-[2-[[(3R,5S)-1-Ethyl-5-hydroxy-3-piperidyl]amino]oxazolo[4,5-b]pyrazin-5-yl]-3-hydroxy-5-methyl-benzonitrile;2,2,2-trifluoroacetic Acid

To a solution of 4-[2-[[(3R,5S)-5-[tert-butyl(diphenyl)silyl]oxy-1-ethyl-3-piperidyl]amino]oxazolo-[4,5-b]pyrazin-5-yl]-3-hydroxy-benzonitrile;2,2,2-trifluoroacetic acid (15.0 mg, 0.02 mmol, 1.0 eq) in THF (1 mL) was added TBAF (0.42 mL, 0.42 mmol, 21.1 eq). Then the mixture was stirred at 20° C. for 16 h. Afterwards, the solvent was removed under vacuum and the residue was purified by preparative HPLC (column: Phenomenex luna C18 150*25 mm*Oum; condition: water (TFA)-MeCN; Begin B: 14, End B: 44; Gradient Time (min): 10, 100% B; Hold Time (min): 3; FlowRate (mL/min): 25) to afford the title compound (3.89 mg, 37% yield) as a white solid. LCMS: m/z 395.4 [M+H]⁺, ESI pos.

### Example 3

### 3-Hydroxy-4-[2-[[(3R)-1-(2-hydroxyethyl)-3-piperidyl]amino]oxazolo[4,5-b]pyridin-5-yl]-5-(methoxymethyl)benzonitrile

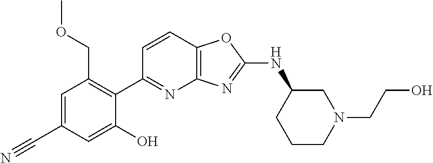 [SMILES: COCc1cc(C#N)cc(O)c1-c1ccc2oc(N[C@@H]3CCCN(CCO)C3)nc2n1]

### Step A: 5-Bromo-N-[(3R)-1-[2-[tert-butyl(dimethyl)silyl]oxyethyl]-3-piperidyl]oxazolo[4,5-b]pyridin-2-amine

To a solution of (3R)-1-[2-[tert-butyl(dimethyl)silyl]oxyethyl]piperidin-3-amine Intermediate 3 (200.0 mg, 0.77 mmol, 1.0 eq) in MeCN (2 mL) was added DIEA (0.26 mL, 1.55 mmol, 2.0 eq), followed by a solution of 5-bromo-2-chloro-oxazolo[4,5-b]pyridine Intermediate 5 (180.6 mg, 0.77 mmol, 1.0 eq) in MeCN (2 mL) and the mixture was stirred at 25° C. for 2 h. Afterwards, the reaction mixture was concentrated under vacuum and the residue was purified by C18 column chromatography (column: Waters Xbridge 150*25 mm*5 um; mobile phase: A for H₂O (0.1% NH₃·H₂O v/v) and B for acetonitrile; Gradient: B 78%-28% in 9 min linearly; Flow rate: 25 mL/mini column; temperature: r.t.; Wavelength: 220 nm/254 nm) to yield the title compound (210.0 mg, 40% yield) as a colorless foam. LCMS m/z 455.1 [M+H]⁺, ESI pos.

### Step B: 4-[2-[[(3R)-1-[2-[tert-Butyl(dimethyl)silyl]oxyethyl]-3-piperidyl]amino]oxazolo[4,5-b]pyridin-5-yl]-3-(methoxymethyl)-5-(2-trimethylsilylethoxymethoxy)benzonitrile

To a solution of 5-bromo-N-[(3R)-1-[2-[tert-butyl(dimethyl)silyl]oxyethyl]-3-piperidyl]oxazolo[4,5-b]pyridin-2-amine (100.0 mg, 0.22 mmol, 1.0 eq) and 3-(methoxymethyl)-4-(4,4,5,5-tetramethyl-1,3,2-dioxaborolan-2-yl)-5-((2-(trimethylsilyl)ethoxy)methoxy)benzonitrile Intermediate 4 (110.5 mg, 0.26 mmol, 1.2 eq) in 1,4-dioxane (1 mL) and water (0.2 mL) was added CsF (133.4 mg, 0.88 mmol, 4.0 eq) and XPhos Pd G₃ (37.2 mg, 0.04 mmol, 0.2 eq), and the reaction mixture was stirred at 95° C. for 2 h under nitrogen atmosphere. Afterwards, the mixture was cooled to room temperature and diluted with ethyl acetate (10 mL), filtered and the filtrate was concentrated in vacuum. The residue was purified by column chromatography on silica gel (PE:EtOAc, PE: 54% to 46%) to afford the title compound (90.0 mg, 61% yield) as a yellow oil. LCMS m/z 668.4 [M+H]⁺, ESI pos.

### Step C: 3-Hydroxy-4-[2-[[(3R)-1-(2-hydroxyethyl)-3-piperidyl]amino]oxazolo[4,5-b]pyridin-5-yl]-5-(methoxymethyl)benzonitrile

To a solution of 4-[2-[[(3R)-1-[2-[tert-butyl(dimethyl)silyl]oxyethyl]-3-piperidyl]amino]oxazolo[4,5-b]pyridin-5-yl]-3-(methoxymethyl)-5-(2-trimethylsilylethoxy-methoxy)benzonitrile (45.0 mg, 0.07 mmol, 1.0 eq) in DCM (1 mL) was added TFA (1.5 mL) and stirring was continued at 20° C. for 2 h. Upon reaction completion, the mixture was diluted with DCM (3 mL) and MeOH (1 mL), and the pH was adjusted to about 8 with NH₃·H₂O, filtered and the filtrate was concentrated under reduced pressure. The crude product was then purified by preparative HPLC (Column: Waters Xbridge 150*25 mm*5 um; Condition: water (0.1% NH₃·H₂O)-MeCN; Begin B 5 End B 35; Gradient Time (min): 10; 100% B Hold Time (min): 2; FlowRate (mL/min) 25) to give the title compound as a off-white solid (25.9 mg, 89% yield). LCMS m/z 424.3 [M+H]⁺, ESI pos.

### Example 4

### 3-Hydroxy-5-methyl-4-[2-[[(3R)-1-(2-oxazol-2-ylethyl)-3-piperidyl]amino]oxazolo[4,5-b]pyridin-5-yl]benzonitrile

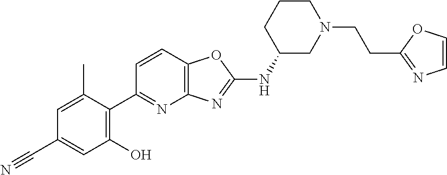 [SMILES: Cc1cc(C#N)cc(O)c1-c1ccc2oc(N[C@@H]3CCCN(CCc4ncco4)C3)nc2n1]

### Step A: tert-Butyl (3R)-3-[(5-chlorooxazolo[4,5-b]pyridin-2-yl)amino]piperidine-1-carboxylate

To a mixture of 5-chloro-2-(methylthio)oxazolo[4,5-b]pyridine (CAS #1783370-92-2, 600 mg, 2.84 mmol, 1.00 eq) in 1,4-dioxane (5.7 mL) was added tert-butyl (3R)-3-aminopiperidine-1-carboxylate (CAS #188111-79-7, 720 mg, 3.59 mmol, 1.27 eq) followed by triethylamine (334 mg, 0.46 mL, 3.30 mmol, 1.16 eq). The brown solution was stirred at 110° C. for 16 hours. The reaction mixture was cooled to room temperature and extracted with ethyl acetate and saturated aq. NaHCO₃-solution. The aqueous layer was back-extracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 40 g, gradient 0% to 70% ethyl acetate in heptane) to afford the title compound (991 mg, 94% yield) as an off-white foam. LCMS: m/z 353.1 [M+H]⁺, ESI pos.

### Step B: 5-Chloro-N-[(3R)-3-piperidyl]oxazolo[4,5-b]pyridin-2-amine

To a solution of tert-butyl (3R)-3-[(5-chlorooxazolo[4,5-b]pyridin-2-yl)amino]piperidine-1-carboxylate (Example 4, step A) (227 mg, 0.61 mmol, 1.00 eq) in dichloromethane (1.8 mL) and methanol (0.90 mL) was added dropwise 4 M HCl in dioxane (1.8 g, 1.5 mL, 6.00 mmol, 9.82 eq). The reaction mixture was stirred at room temperature for 2 hours and then concentrated in vacuo. The residue was dissolved in a small amount of dichloromethane plus a few drops of methanol, then added carefully onto saturated aq. NaHCO₃-solution and extracted three times with a mixture of dichloromethane/methanol (9:1). The combined organic layers were dried over sodium sulfate, filtered and concentrated in vacuo to afford the title compound (137 mg, 88% yield) as an off-white solid. LCMS: m/z 253.0 [M+H]⁺, ESI pos.

### Step C: Methyl 3-[(3R)-3-[(5-chlorooxazolo[4,5-b]pyridin-2-yl)amino]-1-piperidyl]propanoate

To a mixture of 5-chloro-N-[(3R)-3-piperidyl]oxazolo[4,5-b]pyridin-2-amine (Example 4, step B) (68 mg, 0.27 mmol, 1.00 eq) in tetrahydrofuran (1.2 mL) and N,N-dimethylformamide (1.2 mL) was added N,N-diisopropylethylamine (86 mg, 0.116 mL, 0.66 mmol, 2.49 eq) followed by dropwise addition of methyl 3-bromopropanoate (CAS #3395-91-3, 57 mg, 0.037 mL, 0.34 mmol, 1.27 eq). The reaction mixture was stirred at 60° C. for 16 hours. The reaction mixture was cooled to room temperature and then extracted with ethyl acetate and 10% aq. LiCl-solution. The aqueous layer was back-extracted with ethyl acetate. The organic layers were washed two times with 10% aq. LiCl-solution, once with water and once with brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in ethyl acetate) to afford the title compound (74 mg, 78% yield) as a colorless viscous oil. LCMS: m/z 339.1 [M+H]⁺, ESI pos.

### Step D: 3-[(3R)-3-[(5-Chlorooxazolo[4,5-b]pyridin-2-yl)amino]-1-piperidyl]-N-(2,2-dimethoxyethyl)propanamide

To a solution of methyl 3-[(3R)-3-[(5-chlorooxazolo[4,5-b]pyridin-2-yl)amino]-1-piperidyl]propanoate (Example 4, step C) (74 mg, 0.21 mmol, 1.00 eq) in tetrahydrofuran (0.80 mL) and methanol (0.80 mL) was added dropwise lithium hydroxide, 1M aq. solution (0.27 mL, 0.27 mmol, 1.30 eq). The reaction mixture was stirred at room temperature for 16 hours. The reaction mixture was concentrated in vacuo. The residue was taken up twice in toluene, concentrated in vacuo and then put under high vacuum. The residue was suspended in N,N-dimethylformamide (1.0 mL). Aminoacetaldehyde dimethyl acetal (CAS #22483-09-6, 34 mg, 0.035 mL, 0.32 mmol, 1.55 eq) was added followed by N,N-diisopropylethylamine (85 mg, 0.115 mL, 0.66 mmol, 3.17 eq) and HATU (100 mg, 0.26 mmol, 1.27 eq). The reaction mixture was stirred at room temperature for 2.5 hours. The reaction mixture was extracted with ethyl acetate and 5% aq. LiCl-solution. The aqueous layer was backextracted with ethyl acetate. The organic layers were washed three times with 5% aq. LiCl-solution, once with water and once with brine. The combined organic layers were dried over sodium sulfate, filtered and concentrated. The crude material was adsorbed on ISOLUTE HM-N and purified by flash chromatography (silica gel, 12 g, gradient 0% to 5% methanol in dichloromethane) to afford the title compound (56 mg, 62% yield) as a yellow viscous oil. LCMS: m/z 412.1 [M+H]⁺, ESI pos.

### Step E: 5-Chloro-N-[(3R)-1-(2-oxazol-2-ylethyl)-3-piperidyl]oxazolo[4,5-b]pyridin-2-amine

A solution of 3-[(3R)-3-[(5-chlorooxazolo[4,5-b]pyridin-2-yl)amino]-1-piperidyl]-N-(2,2-dimethoxyethyl)propanamide (Example 4, step D) (56 mg, 0.13 mmol, 1.00 eq) in Eaton's reagent (CAS #39394-84-8, 1.24 g, 0.82 mL, 5.22 mmol, 40.4 eq) was stirred at 100° C. for 16 h. The reaction mixture was added dropwise on saturated aq. NaHCO₃-solution (20 mL) and then extracted three times with dichlormethane. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichlormethane and then 10% to 100% (dichloromethane:methanol:NH₄OH 9:1:0.05) in dichlormethane) to afford the title compound (13 mg, 23% yield, 80% purity) as a brown viscous oil. LCMS: m/z 348.1 [M+H]⁺, ESI pos.

### Step F: 3-Hydroxy-5-methyl-4-[2-[[(3R)-1-(2-oxazol-2-ylethyl)-3-piperidyl]amino]oxazolo[4,5-b]pyridin-5-yl]benzonitrile

A mixture of 5-chloro-N-[(3R)-1-(2-oxazol-2-ylethyl)-3-piperidyl]oxazolo[4,5-b]pyridin-2-amine (Example 4, step E) (13 mg, 0.03 mmol, 1.00 eq, 80% purity), (4-cyano-2-hydroxy-6-methyl-phenyl)boronic acid, Intermediate 7, (8 mg, 0.05 mmol, 1.51 eq), cesium carbonate (31 mg, 0.10 mmol, 3.18 eq) and XPhos Pd G₃ (5 mg, 0.01 mmol, 0.20 eq) in 1,4-dioxane (0.30 mL) and water (0.075 mL) was flushed with argon and stirred at 100° C. for 2.5 hours and at room temperature for 16 hours. The reaction mixture was extracted with ethyl acetate and half-saturated aq. NH₄Cl-solution. The aqueous layer was back-extracted with ethyl acetate. The organic layers were washed with water and brine. The combined organic layers were dried over sodium sulfate, filtered and concentrated in vacuo. The crude product was purified by flash chromatography (silica gel, 12 g, gradient 0% to 10% methanol in dichloromethane). All fractions containing product were combined and concentrated in vacuo to afford the title compound (6 mg, 41% yield, 90% purity) as a light yellow solid. LCMS: m/z 445.1 [M+H]⁺, ESI pos.

### Example 5

### 4-[(3S,5R)-3-Hydroxy-5-[[5-[2-hydroxy-6-methyl-4-(trifluoromethyl)phenyl]oxazolo[4,5-b]pyridin-2-yl]amino]-1-piperidyl]butanoic Acid

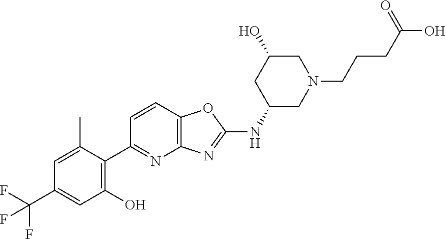 [SMILES: Cc1cc(C(F)(F)F)cc(O)c1-c1ccc2oc(N[C@@H]3C[C@H](O)CN(CCCC(=O)O)C3)nc2n1]

### Step A: Methyl 4-[(3S,5R)-3-hydroxy-5-[[5-[2-hydroxy-6-methyl-4-(trifluoromethyl)phenyl]oxazolo[4,5-b]pyridin-2-yl]amino]-1-piperidyl]butanoate

To a suspension of (3S,5R)-5-[[5-[2-hydroxy-6-methyl-4-(trifluoromethyl)phenyl]oxazolo[4,5-b]pyridin-2-yl]amino]piperidin-3-ol;dihydrochloride (example 6, step C) (90 mg, 0.18 mmol, 1.0 eq) in dichloromethane (4 mL) was added at 0° C. 4-ketobutyric acid methyl ester ((CAS #13865-19-5), 57 mg, 0.05 mL, 0.44 mmol, 2.50 eq) followed by sodium acetate ((CAS #127-09-3), 36 mg, 0.44 mmol, 2.50 eq) and sodium triacetoxyborohydride ((CAS #56553-60-7) 68 mg, 0.32 mmol, 1.80 eq). The reaction mixture was stirred at 0° C. for 5 min and at room temperature for 3 hours. To the reaction mixture, saturated NaHCO₃ solution (30 ml) was added and extracted with dichloromethane (3×50 mL). The organic phase was separated and washed with water (20 ml) and brine (20 mL). The combined organic layers were dried over sodium sulfate, filtered off and concentrated in vacuo. The crude product was purified by flash chromatography (silica gel, 12 g, gradient O % to 50% (dichloromethane:methanol:NH₄OH 110:10:1) in dichloromethane) to afford the title compound (78 mg, 82% yield) as a white foam. LCMS: m/z 507.1 [M−H]⁻, ESI neg.

### Step B: 4-[(3S,5R)-3-hydroxy-5-[[5-[2-hydroxy-6-methyl-4-(trifluoromethyl)phenyl]-oxazolo[4,5-b]pyridin-2-yl]amino]-1-piperidyl]butanoic Acid

To a solution of aforementioned methyl 4-[(3S,5R)-3-hydroxy-5-[[5-[2-hydroxy-6-methyl-4-(trifluoromethyl)phenyl]oxazolo[4,5-b]pyridin-2-yl]amino]-1-piperidyl]butanoate (76 mg, 0.14 mmol, 1.0 eq) in tetrahydrofuran (1.0 mL) and methanol (0.5 mL) was added 1 M aqeuous lithium hydroxide solution (0.28 mL, 0.28 mmol, 2.0 eq). Stirred at 23° C. for 16 hours. All organic solvents were evaporated. The crude product was then purified by preparative HPLC (Column: Gemini NX, 12 nm, 5 μm, 100×30 mm; Condition: MeCN/water+0.10% HCOOH; Gradient: 20-60 MeCN in water, runtime 11 min) to afford the title compound (34 mg, 46% yield) as a white solid. LCMS: m/z 495.2 [M+H]⁺, ESI pos.

### Example 6

### (3S,5R)-1-(2-Hydroxyethyl)-5-[[5-[2-hydroxy-6-methyl-4-(trifluoromethyl)phenyl]oxazolo[4,5-b]pyridin-2-yl]amino]piperidin-3-ol

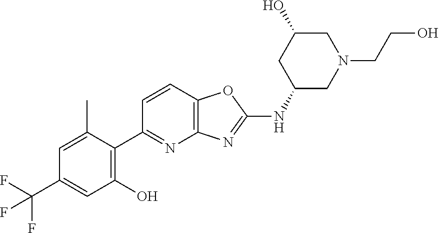 [SMILES: Cc1cc(C(F)(F)F)cc(O)c1-c1ccc2oc(N[C@@H]3C[C@H](O)CN(CCO)C3)nc2n1]

### Step A tert-Butyl (3R,5S)-3-[(5-chlorooxazolo[4,5-b]pyridin-2-yl)amino]-5-hydroxy-piperidine-1-carboxylate

To a mixture of 5-chloro-2-(methylthio)oxazolo[4,5-b]pyridine (CAS #1783370-92-2, 387 mg, 1.83 mmol, 1.0 eq) in 1,4-dioxane (4 mL) was added (3R,5S)-3-amino-5-hydroxy-piperidine-1-carboxylic acid tert-butyl ester (CAS #1932513-59-1, 476 mg, 2.2 mmol, 1.20 eq), followed by triethylamine (278 mg, 0.38 mL, 2.75 mmol, 1.50 eq). The reaction mixture was stirred at 110° C. for 48 hours. The reaction mixture was extracted with ethyl acetate (3×80 mL) and 1 M Na₂CO₃ (80 mL). The organic layers were washed with water (30 mL) and brine (30 mL). The combined organic layers were dried over sodium sulfate, filtered and concentrated in vacuo. The crude product was purified by flash chromatography (silica gel, 12 g, gradient 0% to 50% (dichloromethane:methanol:NH₄OH 110:10:1) in dichloromethane) to afford the title compound (535 mg, 75% yield) as a colorless oil. LCMS: m/z 369.1 ([{35Cl}M+H]⁺), 371.1 ([{37C1}M+H]⁺), ESI pos.

### Step B: tert-Butyl-(3S,5R)-3-hydroxy-5-[[5-[2-hydroxy-6-methyl-4-(trifluoromethyl)phenyl]oxazolo[4,5-b]pyridin-2-yl]amino]piperidine-1-carboxylate

In a sealable tube, a mixture of aforementioned tert-butyl-(3R,5S)-3-[(5-chlorooxazolo[4,5-b]pyridin-2-yl)amino]-5-hydroxy-piperidine-1-carboxylate (step A) (150 mg, 0.39 mmol, 1.0 eq), 3-methyl-2-(4,4,5,5-tetramethyl-1,3,2-dioxaborolan-2-yl)-5-(trifluoromethyl)phenol (163 mg, 0.54 mmol, 1.40 eq) and cesium carbonate (378 mg, 1.16 mmol, 3.0 eq) in 1,4-dioxane (4 mL) and water (1 mL) was set under argon, and xphos-g3-palladacycle (CAS #1445085-55-1, 49 mg, 0.05 mmol, 0.150 eq) was added finally. The reaction mixture was stirred at 90° C. in the sealed tube for 4 hours. The reaction mixture was cooled to room temperature. Quenched with water (20 mL) and saturated NH₄Cl solution (20 mL), then extracted with ethyl acetate (2×40 mL). The organic layers were washed with brine (40 mL), dried over Na₂SO₄, filtered off and concentrated in vacuo. The residue was purified by flash chromatography (silica gel, 12 g, gradient 0% to 50% ethyl acetate in heptane; then ethyl acetate:methanol 9:1 (v/v) to afford the title compound (169 mg, 86% yield) as a light brown foam. LCMS: m/z 509.1 [M+H]⁺, ESI pos.

### Step C: (3S,5R)-5-[[5-[2-hydroxy-6-methyl-4-(trifluoromethyl)phenyl]oxazolo[4,5-b]pyridin-2-yl]amino]piperidin-3-ol;dihydrochloride

To a solution of tert-butyl-(3S,5R)-3-hydroxy-5-[[5-[2-hydroxy-6-methyl-4-(trifluoromethyl)phenyl]oxazolo[4,5-b]pyridin-2-yl]amino]piperidine-1-carboxylate (step B) (169 mg, 0.33 mmol, 1.0 eq) in dichloromethane (8 mL) and methanol (2 mL) was added dropwise at ambient temperature 4 M HCl in 1,4-dioxane (997 mg, 0.83 mL, 3.32 mmol, 10.0 eq). The reaction mixture was stirred at 23° C. for 16 hours. Evaporation of the reaction mixture afforded the title compound (158 mg, 94%) as a light yellow foam. LCMS: m/z 409.1 [M+H]⁺, ESI pos.

### Step D: (3S,5R)-1-[2-[tert-butyl(dimethyl)silyl]oxyethyl]-5-[[5-[2-hydroxy-6-methyl-4-(trifluoromethyl)phenyl]oxazolo[4,5-b]pyridin-2-yl]amino]piperidin-3-ol

To a suspension of (3S,5R)-5-[[5-[2-hydroxy-6-methyl-4-(trifluoromethyl)phenyl]oxazolo[4,5-b]pyridin-2-yl]amino]piperidin-3-ol;dihydrochloride (step C) (68 mg, 0.13 mmol, 1.0 eq) in dichloromethane (3 mL) was added at 0° C. 2-((tert-butyldimethylsilyl)oxy)acetaldehyde (CAS #102191-92-4, 65 mg, 0.07 mL, 336 mmol, 2.50 eq) followed by sodium acetate ((CAS #127-09-3), 28 mg, 0.34 mmol, 2.50 eq) and sodium triacetoxyborohydride (CAS #56553-60-7, 51 mg, 0.24 mmol, 1.80 eq). The reaction mixture was stirred at 0° C. for 5 min and at 23° C. for 3 hours. To the reaction mixture, saturated NaHCO₃ solution (30 mL) was added and extracted with dichloromethane (3×50 mL). The organic phase was separated and washed with water (20 ml) and brine (20 ml). The combined organic layers were dried over sodium sulfate, filtered off and concentrated in vacuo to afford the title compound (170 mg; 98% yield) as a light brown oil. LCMS: m/z 565.2 [M−H]⁻, ESI neg.

### Step E: (3S,5R)-1-(2-hydroxyethyl)-5-[[5-[2-hydroxy-6-methyl-4-(trifluoromethyl)phenyl]oxazolo[4,5-b]pyridin-2-yl]amino]piperidin-3-ol

Dropwise addition of 1 M tetrabutylammonium fluoride in THF (CAS #429-41-4, 234 mg, 0.26 mL, 0.26 mmol, 2.0 eq) to a solution (3S,5R)-1-[2-[tert-butyl(dimethyl)silyl]oxyethyl]-5-[[5-[2-hydroxy-6-methyl-4-(trifluoromethyl)phenyl]oxazolo[4,5-b]pyridin-2-yl]amino]piperidin-3-ol (step D) (170 mg, 0.13 mmol, 1.0 eq) in tetrahydrofuran (3.52 mL). The reaction mixture was stirred at 23° C. for 2 hours. The reaction mixture was extracted with ethyl acetate (2×30 mL).

The organic layers were washed with water (30 mL) and brine (30 mL). The combined organic extracts were dried over sodium sulfate, filtered off and evaporated. The crude product was then purified by preparative HPLC (Column: Gemini NX, 12 nm, 5 μm, 100×30 mm; Condition: MeCN/water+0.1% TEA; Gradient: 5-10-100 MeCN in water, runtime 15 min) to afford the title compound (24 mg, 38% yield) as a white solid. LCMS: m/z 453.3 [M+H]⁺, ESI pos.

### Example 7

### 3-Hydroxy-4-[2-[[(3R,5S)-5-hydroxy-1-methyl-3-piperidyl]amino]oxazolo[4,5-b]pyrazin-5-yl]-5-methyl-benzonitrile;2,2,2-trifluoroacetic Acid

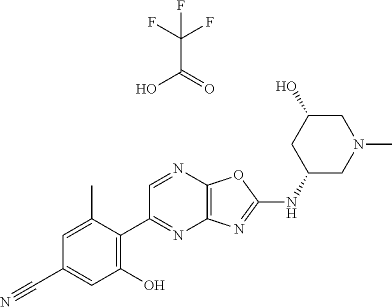 [SMILES: Cc1cc(C#N)cc(O)c1-c1cnc2oc(N[C@@H]3C[C@H](O)CN(C)C3)nc2n1 ; O=C(O)C(F)(F)F]

### Step A: 4-(5-(Benzyloxy)-6-isothiocyanatopyrazin-2-yl)-3-methyl-5-((2-(trimethylsilyl)ethoxy) methoxy)benzonitrile

To a solution of 1,1′-thiocarbonyldipyridin-2(1H)-one (773 mg, 3.33 mmol, 1.1 eq) in DCM (16 mL) was added 4-(6-amino-5-benzyloxy-pyrazin-2-yl)-3-methyl-5-(2-trimethylsilylethoxy methoxy)benzonitrile (1400 mg, 3.03 mmol, 1.0 eq). The reaction mixture was stirred at 25° C. for 2 hours. Upon the reaction completion, the reaction mixture was concentrated under vacuum and purified by column chromatography on silica gel (petroleum ether/ethyl acetate 20:1 to 10:1) to give the title compound as yellow oil. LCMS: m/z 505.2 [M+H]⁺, ESI pos.

### Step B: tert-Butyl (3R,5S)-3-(3-(3-(enzyloxy)-6-(4-cyano-2-methyl-6-((2-(trimethylsilyl)ethoxy)methoxy)phenyl)pyrazin-2-yl)thioureido)-5-hydroxypiperidine-1-carboxylate

To a solution of tert-butyl (3R,5S)-3-amino-5-hydroxy-piperidine-1-carboxylate (157 mg, 0.73 mmol, 1.05 eq) and DIEA (0.18 mL, 1.04 mmol, 1.5 eq) in DCM (5 mL) was dropwise added a solution of 4-(5-benzyloxy-6-isothiocyanato-pyrazin-2-yl)-3-methyl-5-(2-trimethylsilylethoxymethoxy)benzonitrile (350 mg, 0.69 mmol, 1.0 eq) in DCM (5 mL). The reaction mixture was stirred at 20° C. for 2 h. Upon the reaction completion, the reaction mixture was concentrated under vacuum to give a residue. The residue was purified by column chromatography on silica gel (petroleum ether/ethyl acetate 10:1 to 2:1) to afford the title compound (450 mg, 90% yield) as a yellow oil. LCMS: m/z 721.3 [M+H]⁺, ESI pos.

### Step C: 1-(6-(4-Cyano-2-hydroxy-6-methylphenyl)-3-hydroxypyrazin-2-yl)-3-((3R,5S)-5-hydroxypiperidin-3-yl)thiourea

A solution of tert-butyl (3R,5S)-3-[[3-benzyloxy-6-[4-cyano-2-methyl-6-(2-trimethylsilylethoxymethoxy)phenyl]pyrazin-2-yl]carbamothioylamino]-5-hydroxy-piperidine-1-carboxy late (450 mg, 0.62 mmol, 1.0 eq) in TFA (4.5 mL, 60.49 mmol, 97 eq) was stirred at 25° C. for 2 h. Upon the reaction completion, the reaction mixture was concentrated under vacuum and dissolved in MeCN (1 mL), and the pH was adjusted to pH=7-8 by addition of NH₃·H₂O. The residue was purified by C₁₈ column chromatography (20 g, 0.1% NH₃·H₂O in water/MeCN, MeCN: 30%-40%) to give the title compound (180 mg, 72% yield) as white solid. ¹H NMR (400 MHz, CD₃OD) δ [ppm]: 7.16-7.13 (m, 1H), 7.07 (s, 1H), 7.04 (s, 1H), 4.48-4.36 (m, 1H), 3.76-3.64 (m, 1H), 3.27 (m, 1H), 3.08-2.94 (m, 1H), 2.57-2.45 (m, 1H), 2.44-2.36 (m, 1H), 2.33 (s, 1H), 2.29 (s, 3H), 1.39-1.33 (m, 1H).

### Step D: 1-(6-(4-Cyano-2-hydroxy-6-methylphenyl)-3-hydroxypyrazin-2-yl)-3-((3R,5S)-5-hydroxy-1-methylpiperidin-3-yl)thiourea

To a solution of 1-[6-(4-cyano-2-hydroxy-6-methyl-phenyl)-3-hydroxy-pyrazin-2-yl]-3-[(3R,5S)-5-hydroxy-3-piperidyl]thiourea (100 mg, 0.25 mmol, 1.0 eq) in methanol (1 mL)/DCE (5 mL) was added formaldehyde (61 mg, 0.75 mmol, 3.0 eq) and stirred at 20° C. for 0.5 h, then NaBH(OAc)₃ (132 mg, 0.62 mmol, 2.5 eq) was added. The reaction mixture was stirred at 20° C. for 2 hours. Upon the reaction completion, the reaction mixture was concentrated in vacuum. The residue was purified by reversed phase flash (CombiFlash 0.1% TFA aqueous-MeCN condition) tion to give the title compound (100 mg, 76% yield) as yellow solid. LCMS: m/z 415.2 [M+H]⁺, ESI pos.

### Step E: 3-Hydroxy-4-[2-[[(3R,5S)-5-hydroxy-1-methyl-3-piperidyl]amino]oxazolo[4,5-b]pyrazin-5-yl]-5-methyl-benzonitrile;2,2,2-trifluoroacetic Acid

To a solution of 1-[6-(4-cyano-2-hydroxy-6-methyl-phenyl)-3-hydroxy-pyrazin-2-yl]-3-[(3R,5S)-5-hydroxy-1-methyl-3-piperidyl]thiourea;2,2,2-trifluoroacetic acid (100 mg, 0.19 mmol, 1.0 eq) and tetrabutylazanium; iodide (84 mg, 0.23 mmol, 1.2 eq) in THF (2 mL) was dropwise added H₂O₂ (35 mg, 0.38 mmol, 2.00 eq) and stirred at 20° C. for 0.5 hour. Upon the reaction completion, the reaction mixture was quenched with saturated Na₂SO₃ aqueous solution (1 mL) and then concentrated in vacuum. The residue was purified by reversed phase flash (CombiFlash 0.1% NH₃·H₂O aqueous-MeCN condition) to give crude product (60 mg). Then it was repurified by preparative-HPLC (Column: Phenomenex luna C₁₈ 150*25 mm*10 um; Condition; water(TFA)-MeCN; Begin: B, 14, End B: 44; Gradient Time (min): 10; 100% B Hold Time (min): 3; FlowRate (ml/min): 25.) to afford the title compound (22.1 mg, 23% yield) as a white solid. LCMS: m/z 381.1 m/z 395.1 [M+H]⁺, ESI pos.

### Example 8

### 3-Hydroxy-4-[2-[[(3R)-1-(2-hydroxyethyl)-3-piperidyl]amino]-7-methoxy-oxazolo[4,5-b]pyridin-5-yl]-5-methyl-benzonitrile;2,2,2-trifluoroacetic Acid

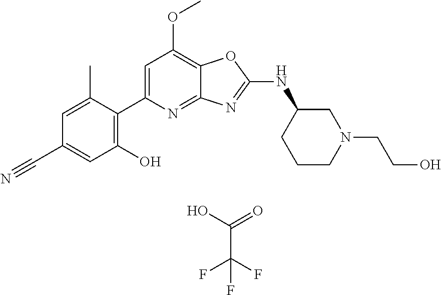 [SMILES: COc1cc(-c2c(C)cc(C#N)cc2O)nc2nc(N[C@@H]3CCCN(CCO)C3)oc12 ; O=C(O)C(F)(F)F]

### Step A: 6-Chloro-4-iodo-2-nitropyridin-3-ol

In a three round bottom flask, 6-chloro-4-iodo-pyridin-3-ol (5.0 g, 19.57 mmol, 1.0 eq, CAS #877133-58-9) in acetic acid (50 mL) was added fuming nitric acid (2.65 mL, 63.5 mmol, 3.24 eq) at 20° C., then the mixture was stirred at 20° C. for 2 h. Upon the reaction completion, the reaction mixture was slowly pipetted into ice water (150 mL). EtOAc (150 mL) was added and layers were separated. The aqueous phase was extracted with EtOAc (3×150 mL). Combined extracts were washed with sat. NaHCO₃ (3×100 mL), dried over Na₂SO₄, filtered, and concentrated under vacuum to give a residue which was purified by column chromatography on silica gel (petroleum ether:ethyl acetate=5:1 to 2:1) to afford the title compound (2.90 g, 49% yield) as a yellow solid. LCMS: m/z 298.9 [M−H]⁻, ESI neg.

### Step B: 6-Chloro-4-methoxy-2-nitropyridin-3-ol

To a solution of 6-chloro-4-iodo-2-nitro-pyridin-3-ol (1.3 g, 4.33 mmol, 1.0 eq) in methanol (13 mL) was added sodium methoxide (4.95 mL, 21.63 mmol, 5.0 eq) and CuI (0.99 g, 5.19 mmol, 1.2 eq). The reaction mixture was stirred at 80° C. for 3 h. Upon reaction completion, the reaction mixture was cooled to room temperature and water (30 mL) was added, acidified to pH=5-6 by 1N HCl. EtOAc (50 mL) was added and layers were separated. The aqueous phase was extracted with EtOAc (3×50 mL). Combined extracts were washed with brine (3×30 mL), dried over Na₂SO₄, filtered, and concentrated under vacuum to give a residue which was purified by column chromatography on silica gel (petroleum ether:ethyl acetate=3:1 to 2:1) to the title compound (500.0 mg, 56% yield) as a yellow solid. ¹H NMR (400 MHz, DMSO-d₆) δ [ppm]: 11.43-11.14 (m, 1H), 7.48 (s, 1H), 4.00 (s, 3H).

### Step C: 2-Amino-6-chloro-4-methoxypyridin-3-ol

To a solution of 6-chloro-4-methoxy-2-nitro-pyridin-3-ol (100.0 mg, 0.49 mmol, 1.0 eq) in methanol (1 mL) was added tin(II) chloride (278.06 mg, 1.47 mmol, 3.0 eq). The reaction mixture was stirred at 70° C. for 2 h. Upon the reaction completion, the reaction mixture was cooled to room temperature and water was added (20 mL), basified to pH=7 by NaHCO₃. EtOAc (20 mL) were added and layers were separated. The aqueous phase was extracted with EtOAc (3×20 mL). Combined extracts were washed with brine (2×10 mL), dried over Na₂SO₄, filtered, and concentrated under vacuum to give a residue which was purified by column chromatography on silica gel (petroleum ether:ethyl acetate=1:1 to 1:2) to afford the title compound (45.0 mg, 53% yield) as a white solid. LCMS: m/z 175.0 [M+H]⁺, ESI pos.

### Step D: 5-Chloro-7-methoxyoxazolo [4, 5-b]pyridine-2-thiol

To a solution of 2-amino-6-chloro-4-methoxy-pyridin-3-ol (40.0 mg, 0.23 mmol, 1.0 eq) in DMF (0.5 mL) was added di(1H-imidazol-1-yl)methanethione (40.83 mg, 0.23 mmol, 1.0 eq) and stirred at 20° C. for 16 hours. Upon reaction completion, the reaction mixture was quenched with water (10 mL) and acidified to pH=2-3 by 1N HCl. EtOAc (10 mL) and water (10 mL) were added and layers were separated. The aqueous phase was extracted with EtOAc (3×10 mL). Combined extracts were washed with sat. CaCl₂ solution (2×10 mL), dried over Na₂SO₄, filtered, and concentrated under vacuum to afford the title compound (30.0 mg, 60% yield) as a yellow solid. ¹H NMR (400 MHz, DMSO-d₆) δ [ppm]: 7.12 (s, 1H), 4.04 (s, 3H).

### Step E: 2, 5-Dichloro-7-methoxyoxazolo [4, 5-b]pyridine

To a mixture of 5-chloro-7-methoxy-oxazolo[4,5-b]pyridine-2-thiol (30.0 mg, 0.14 mmol, 1.0 eq) in oxalyl chloride (1.0 mL, 11.8 mmol, 85 eq) was added DMF (1.01 mg, 0.01 mmol, 0.1 eq), then the mixture was stirred at 50° C. for 1 h. Upon reaction completion, the reaction mixture was cooled to 20° C. and then concentrated in vacuum, ice water (5 mL) was added, extracted with EtOAc (2×10 mL). Combined extracts were washed with brine (10 mL), dried over Na₂SO₄, filtered, and concentrated under vacuum to give a residue, which was purified by column chromatography on silica gel (petroleum ether:ethyl acetate=10:1 to 8:1) to afford the title compound (16.0 mg, 53% yield) as a yellow solid. LCMS: m/z 218.9 [M+H]⁺, ESI pos.

### Step F: (R)—N-(1-(2-((tert-butyldimethylsilyl)oxy)ethyl)piperidin-3-yl)-5-chloro-7-methoxyoxazolo [4,5-b]pyridin-2-amine, also known as N-[(3R)-1-[2-[tert-butyl(dimethyl)silyl]oxyethyl]-3-piperidyl]-5-chloro-7-methoxy-oxazolo[4,5-b]pyridin-2-amine

To a solution of (3R)-1-[2-[tert-butyl(dimethyl)silyl]oxyethyl]piperidin-3-amine (21.2 mg, 0.08 mmol, 1.2 eq) and N-ethyl-N-isopropylpropan-2-amine (17.67 mg, 0.14 mmol, 2.0 eq) in acetonitrile (1 mL) was added a solution of 2,5-dichloro-7-methoxy-oxazolo[4,5-b]pyridine (15.0 mg, 0.07 mmol, 1.0 eq) in acetonitrile (0.5 mL) dropwise at 20° C. Then the mixture was stirred at 20° C. for 1 h. Upon reaction completion, the mixture was concentrated in vacuum to give the residue, which was purified by prep-TLC (EtOAc:MeOH=10:1) to afford the title compound (15.0 mg, 50% yield) as yellow oil. ¹H NMR (400 MHz, CD₃OD) δ [ppm]: 6.79 (s, 1H), 4.05 (s, 3H), 3.96-3.88 (m, 1H), 3.82 (t, 2H), 3.22-3.14 (m, 1H), 2.86-2.77 (m, 1H), 2.66-2.58 (m, 2H), 2.36-2.24 (m, 2H), 2.05-1.96 (m, 1H), 1.87-1.77 (m, 1H), 1.73-1.63 (m, 1H), 1.53-1.42 (m, 1H), 0.87 (s, 9H), 0.07 (s, 6H).

### Step G: (R)—N-(1-(2-((tert-butyldimethylsilyl)oxy)ethyl)piperidin-3-yl)-5-chloro-7-methoxyoxazolo [4,5-b]pyridin-2-amine

To a solution of N-[(3R)-1-[2-[tert-butyl(dimethyl)silyl]oxyethyl]-3-piperidyl]-5-chloro-7-methoxy-oxazolo[4,5-b]pyridin-2-amine (14.0 mg, 0.03 mmol, 1.0 eq) and 3-methyl-4-(4,4,5,5-tetramethyl-1,3,2-dioxaborolan-2-yl)-5-(2-trimethylsilylethoxymethoxy)benzonitrile (14.8 mg, 0.04 mmol, 1.2 eq), K₂CO₃ (10.97 mg, 0.08 mmol, 2.5 eq) in 1,4-dioxane (0.5 mL) and water (0.1 mL) was added Xphos Pd G₃ (5.38 mg, 0.01 mmol, 0.2 eq) and stirred at 90° C. for 4 h. Upon reaction completion, the reaction mixture was cooled to room temperature, concentrated under vacuum to give a residue, which was purified by preparative TLC (EtOAc/MeOH=10:1) to afford the title compound (12.0 mg, 57% yield) as colorless oil. ¹H NMR (400 MHz, CD₃OD) δ [ppm]: 7.38 (s, 1H), 7.32 (s, 1H), 6.66 (s, 1H), 5.17 (s, 2H), 4.06 (s, 3H), 3.98-3.91 (m, 1H), 3.81 (t, 2H), 3.59 (t, 2H), 3.24-3.17 (m, 1H), 2.83-2.77 (m, 1H), 2.63-2.56 (m, 2H), 2.33-2.22 (m, 2H), 2.13 (s, 3H), 2.05-2.00 (m, 1H), 1.85-1.78 (m, 1H), 1.74-1.65 (m, 1H), 1.48 (m, 1H), 0.87 (s, 8H), 0.84 (m, 2H), 0.07 (d, 6H), −0.05 (s, 9H).

### Step H: 3-Hydroxy-4-[2-[[(3R)-1-(2-hydroxyethyl)-3-piperidyl]amino]-7-methoxy-oxazolo[4,5-b]pyridin-5-yl]-5-methyl-benzonitrile;2,2,2-trifluoroacetic Acid

A solution of 4-[2-[[(3R)-1-[2-[tert-butyl(dimethyl)silyl]oxyethyl]-3-piperidyl]amino]-7-methoxy-oxazolo [4,5-b]pyridin-5-yl]-3-methyl-5-(2-trimethylsilylethoxymethoxy) benzonitrile (10.0 mg, 0.01 mmol, 1.0 eq) in TFA (0.83 mL, 11.2 mmol, 748 eq) was stirred at 40° C. for 2 hours. Upon reaction completion, the reaction mixture was cooled to room temperature and concentrated under vacuum to give a residue which was purified by preparative HPLC (Column Phenomenex luna C18 150*25 mm*10 um; Condition: water(TFA)-MeCN; Begin B: 14; End B: 34; Gradient Time (min): 7; 100% B Hold Time (min): 3; FlowRate (ml/min): 25.) to afford the title compound (3.9 mg, 48% yield) as a white solid. LCMS: m/z 424.2 [M+H]⁺, ESI pos.

### Example 9

### 3-Hydroxy-4-[2-[[(3R,5S)-5-hydroxy-3-piperidyl]amino]oxazolo[4,5-b]pyridin-5-yl]-5-methyl-benzonitrile

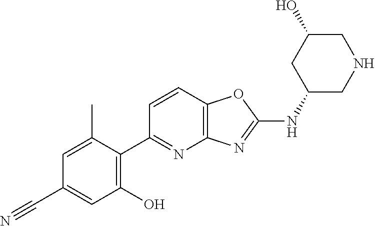 [SMILES: Cc1cc(C#N)cc(O)c1-c1ccc2oc(N[C@H]3CNC[C@@H](O)C3)nc2n1]

### Step A: tert-Butyl (3R,5S)-3-[(5-chlorooxazolo[4,5-b]pyridin-2-yl)amino]-5-hydroxy-piperidine-1-carboxylate

To a mixture of 5-chloro-2-(methylthio)oxazolo[4,5-b]pyridine (CAS #1783370-92-2, 150 mg, 0.71 mmol, 1.00 eq) in 1,4-dioxane (1.4 mL) was added tert-butyl (3R,5S)-3-amino-5-hydroxy-piperidine-1-carboxylate (CAS #1932513-59-1, 177 mg, 0.82 mmol, 1.15 eq), followed by triethylamine (83 mg, 0.115 mL, 0.83 mmol, 1.16 eq). The reaction mixture was stirred at 110° C. for 16 hours. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane) to afford the title compound (251 mg, 86% yield, 90% purity) as a light yellow oil. LCMS: m/z 369.1 [M+H]⁺, ESI pos.

### Step B: tert-Butyl (3R,5S)-3-[[5-(4-cyano-2-hydroxy-6-methyl-phenyl)oxazolo[4,5-b]pyridin-2-yl]amino]-5-hydroxy-piperidine-1-carboxylate

A mixture of tert-butyl (3R,5S)-3-[(5-chlorooxazolo[4,5-b]pyridin-2-yl)amino]-5-hydroxy-piperidine-1-carboxylate (Example 9, step A) (251 mg, 0.61 mmol, 1.00 eq, 90% purity), (4-cyano-2-hydroxy-6-methyl-phenyl)boronic acid, Intermediate 7, (164 mg, 0.93 mmol, 1.51 eq), cesium carbonate (635 mg, 1.95 mmol, 3.18 eq) and XPhos Pd G₃ (102 mg, 0.12 mmol, 0.20 eq) in 1,4-dioxane (6.0 mL) and water (1.5 mL) was flushed with argon and stirred at 100° C. for 3 hours. The reaction mixture was cooled to room temperature and extracted with ethyl acetate and half-saturated aq. NH₄Cl-solution. The aqueous layer was back-extracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% ethyl acetate in heptane to afford the title compound (188 mg, 63% yield) as an off-white solid. LCMS: m z 466.3 [M+H]⁺, ESI pos.

### Step C: 3-Hydroxy-4-[2-[[(3R,5S)-5-hydroxy-3-piperidyl]amino]oxazolo[4,5-b]pyridin-5-yl]-5-methyl-benzonitrile

To a solution of tert-Butyl (3R,5S)-3-[[5-(4-cyano-2-hydroxy-6-methyl-phenyl)oxazolo[4,5-b]pyridin-2-yl]amino]-5-hydroxy-piperidine-1-carboxylate (Example 9, step B) (188 mg, 0.38 mmol, 1.00 eq) in dichloromethane (3 mL) was added dropwise at 0° C. trifluoroacetic acid (992 mg, 0.67 mL, 8.70 mmol, 22.7 eq). Then the reaction mixture was stirred at room temperature for 1 hour. The reaction mixture was concentrated in vacuo. The residue was dissolved in dichlormethane/methanol and carefully basified with saturated aq. NaHCO₃-solution and extracted three times with a mixture of dichlormethane/methanol (9:1). The combined organic layers were dried over sodium sulfate, filtered and concentrated in vacuo. The crude product was purified by SFC (Column chiral IH, eluent B: 20% methanol+0.2% diethylamine) to afford the title compound (25 mg, 17% yield) as a white powder. LCMS: m/z 366.1 [M+H]⁺, ESI pos.

### Example 10

### 3-Hydroxy-4-[2-[[(3R)-1-(2-hydroxyethyl)-3-piperidyl]amino]oxazolo[4,5-b]pyrazin-5-yl]-5-methyl-benzonitrile

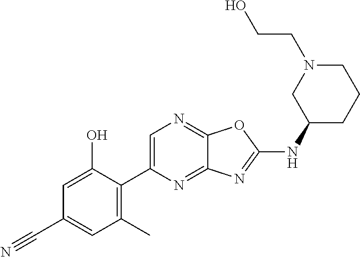 [SMILES: Cc1cc(C#N)cc(O)c1-c1cnc2oc(N[C@@H]3CCCN(CCO)C3)nc2n1]

### Step A: tert-Butyl (3R)-3-[(3-benzyloxy-6-bromo-pyrazin-2-yl)carbamothioylamino]piperidine-1-carboxylate

To a solution of 3-benzyloxy-6-bromo-pyrazin-2-amine (2700.0 mg, 9.64 mmol, 1.0 eq) in THF (15 mL) was added NaH (578.32 mg, 14.46 mmol, 1.5 eq) and stirred for 20 mins, then a solution of tert-butyl (3R)-3-isothiocyanatopiperidine-1-carboxylate (CAS #2824986-69-6, 2803.02 mg, 11.6 mmol, 1.2 eq) in THF (5 mL) was added dropwise and stirring was continued at 20° C. for 4 h under N₂. Upon reaction completion, the mixture was quenched with NH₄Cl (60 mL), and extracted with EtOAc (100 mL×2), the organic phase was washed with brine (100 mL×2), dried over anhydrous Na₂SO₄, filtered and concentrated in vacuum. The residue was purified by column chromatography (SiO₂, PE:EtOAc, 87% to 13%) to give the title compound (3700.0 mg, 73% yield) as a yellow oil. LCMS (de-BOC) m/z: 466.1 [M+H]⁺, ESI pos.

### Step B: 1-(6-Bromo-3-hydroxy-pyrazin-2-yl)-3-[(3R)-3-piperidyl]thiourea

To a solution of tert-butyl (3R)-3-[(3-benzyloxy-6-bromo-pyrazin-2-yl)carbamothioylamino]piperidine-1-carboxylate (3700.0 mg, 7.08 mmol, 1.0 eq) in anisole (20 mL) was added TFA (20 mL) stirred at 70° C. for 2 h under N₂. Upon the reaction completion, the mixture was concentrated in vacuum, diluted with MeOH (6 mL) was adjusted pH to 8.0. The residue was purified by C₁₈ column chromatography (0.1% NH3·H2O-Me CN=54%˜46%), the eluent was lyophilized to give the title compound (1400.0 mg, 60% yield) as a white solid. LCMS m/z: 334.0 [M+2+H]⁺, ESI pos.

### Step C: 1-(6-Bromo-3-hydroxy-pyrazin-2-yl)-3-[(3R)-1-[2-[tert-butyl(dimethyl)silyl]oxyethyl]-3-piperidyl]thiourea

To a solution of 1-(6-bromo-3-hydroxy-pyrazin-2-yl)-3-[(3R)-3-piperidyl]thiourea (570.0 mg, 1.72 mmol, 1.0 eq) in DCE (10 mL) and methanol (1 mL) was added CH₃COOH (10.3 mg, 0.17 mmol, 0.1 eq) stirred at 20 min then NaBH(OAc)₃ (1.82 g, 8.58 mmol, 5.0 eq) was added and the mixture stirred at 20° C. 1 h. Upon reaction completion, the mixture was quenched with H₂O (30 mL), and extracted with EtOAc (30 mL×2), the organic phase was washed with brine (30 mL×2), dried over anhydrous Na₂SO₄, filtered and concentrated in vacuum. The residue was purified by column chromatography (SiO₂, petroleum ether:ethyl acetate, 61% to 39%) to yield the title compound (540.0 mg, 64% yield) as yellow oil. LCMS m/z: 492.2 [M+2+H]⁺, ESI pos.

### Step D: 5-Bromo-N-[(3R)-1-[2-[tert-butyl(dimethyl)silyl]oxyethyl]-3-piperidyl]oxazolo[4,5-b]pyrazin-2-amine

To a solution of 1-(6-bromo-3-hydroxy-pyrazin-2-yl)-3-[(3R)-1-[2-[tert-butyl(dimethyl)silyl]oxyethyl]-3-piperidyl]thiourea (540.0 mg, 1.1 mmol, 1.0 eq) in THF (20 mL) was added tetrabutylammonium iodide (406.6 mg, 1.1 mmol, 1.0 eq) then dropwise H₂O₂ (390.0 mg, 3.44 mmol, 3.13 eq) and stirred at 25° C. for 1 h. Upon reaction completion, the mixture was quenched with H₂O (30 mL) and Na₂SO₃ (30 mL), and extracted with EtOAc (20 mL×2), the organic phase was washed with Na₂SO₃ (30 mL), dried over anhydrous Na₂SO₄, filtered and concentrated in vacuum. The residue was purified by column chromatography (SiO2, petroleum ether:ethyl acetate, 65% to 35%) to give the title compound (169.0 mg, 34% yield) as yellow oil. LCMS m/z: 456.3 [M+H]⁺, ESI pos.

### Step E: 4-[2-[[(3R)-1-[2-[tert-butyl(dimethyl)silyl]oxyethyl]-3-piperidyl]amino]oxazolo[4,5-b]pyrazin-5-yl]-3-methyl-5-(2-trimethylsilylethoxymethoxy)benzonitrile

To a solution of 5-bromo-N-[(3R)-1-[2-[tert-butyl(dimethyl)silyl]oxyethyl]-3-piperidyl]oxazolo[4,5-b]pyrazin-2-amine (269.0 mg, 0.59 mmol, 1.0 eq) and 3-methyl-4-(4,4,5,5-tetramethyl-1,3,2-dioxaborolan-2-yl)-5-(2-trimethylsilylethoxymethoxy)benzonitrile (344.2 mg, 0.88 mmol, 1.5 eq) in 1,4-dioxane (4 mL) and water (0.8 mL) was added CsF (358.08 mg, 2.36 mmol, 4.0 eq) and XPhos Pd G₃ (99.9 mg, 0.12 mmol, 0.2 eq), then stirred at 95° C. for 2 h under N₂. Upon reaction completion, the above reaction was concentrated reduce in pressure, diluted with EtOAc (3 mL) filtered and the filtrate was concentrated in vacuum. The residue was purified by column chromatography (SiO₂, PE:EtOAc=1:1) to give the title compound (70.0 mg, 19% yield) as a yellow oil. LCMS m/z: 639.5 [M+H]⁺, ESI pos. Furthermore, these compounds were also isolated: 2-[(3R)-3-[(5-bromooxazolo[4,5-b]pyrazin-2-yl)amino]-1-piperidyl]ethanol (30.0 mg, 9.7% yield) as a yellow oil. LCMS m/z: 342.1 [M+H]⁺, ESI pos.; as well as 4-[2-[[(3R)-1-(2-hydroxyethyl)-3-piperidyl]amino]oxazolo[4,5-b]pyrazin-5-yl]-3-methyl-5-(2-trimethylsilylethoxymethoxy)benzonitrile (30.0 mg, 10% yield) as a yellow solid. LCMS m/z: 525.3 [M+H]⁺, ESI pos.

### Step F: 4-[2-[[(3R)-1-(2-hydroxyethyl)-3-piperidyl]amino]oxazolo[4,5-b]pyrazin-5-yl]-3-methyl-5-(2-trimethylsilylethoxymethoxy)benzonitrile

To a solution of aforementioned 2-[(3R)-3-[(5-bromooxazolo[4,5-b]pyrazin-2-yl)amino]-1-piperidyl]ethanol (30.0 mg, 0.09 mmol, 1.0 eq) and 3-methyl-4-(4,4,5,5-tetramethyl-1,3,2-dioxaborolan-2-yl)-5-(2-trimethylsilylethoxymethoxy)benzonitrile (51.2 mg, 0.13 mmol, 1.5 eq) in 1,4-Dioxane (1 mL) and water (0.2 mL) was added CsF (53.27 mg, 0.35 mmol, 4.0 eq) and xphos Pd g3 (14.9 mg, 0.02 mmol, 0.2 eq), then stirred at 95° C. for 2 h under N₂. Upon the reaction completion, the above reaction was concentrated reduce in pressure, diluted with EtOAc (2 mL) filtered and the filtrate was concentrated in vacuum. The residue was purified by prep-TLC (EA:MeOH=3:1) to give the title compound (10.0 mg, 22% yield) as yellow oil.

### Step G: 3-Hydroxy-4-[2-[[(3R)-1-(2-hydroxyethyl)-3-piperidyl]amino]oxazolo[4,5-b]pyrazin-5-yl]-5-methyl-benzonitrile

To a solution of 4-[2-[[(3R)-1-(2-hydroxyethyl)-3-piperidyl]amino]oxazolo[4,5-b]pyrazin-5-yl]-3-methyl-5-(2-trimethylsilylethoxymethoxy)benzonitrile (10.0 mg, 0.02 mmol, 1.0 eq) in DCM (0.5 mL) was added TFA (0.5 mL) stirred at 40° C. for 2 hours. Upon reaction completion, the mixture dilute with DCM (2 mL) and MeOH (1 mL), and the pH was adjusted to about 8 with NH3·H2O, then filtered and the filtrate was concentrated under reduced pressure. The crud product purified by prep-HPLC (Column: Waters Xbridge 150*25 mm*5 um; Condition: water(0.1% NH3·H2O)-ACN, Begin B 7 End B 38; Gradient Time (min): 10; 100% B Hold Time (min): 2; FlowRate (mL/min) 25), to give the title compound (2.12 mg, 27% yield) as a white solid. LCMS m/z: 395.2 [M+H]⁺, ESI pos.

### Example 11

### 5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol

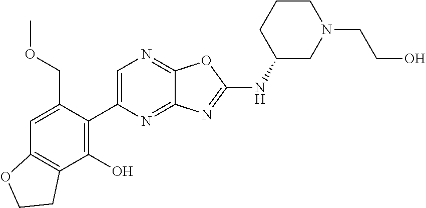 [SMILES: COCc1cc2c(c(O)c1-c1cnc3oc(N[C@@H]4CCCN(CCO)C4)nc3n1)CCO2]

### Step A: 2-[(3R)-3-[(5-Bromooxazolo[4,5-b]pyrazin-2-yl)amino]piperidino]ethanol;2,2,2-trifluoroacetic Acid

A solution of 5-bromo-N-[(3R)-1-[2-[tert-butyl(dimethyl)silyl]oxyethyl]-3-piperidyl]oxazolo[4,5-b]pyrazin-2-amine (290.0 mg, 0.64 mmol, 1.0 eq, Example 10, Step D) in TFA (3.0 mL, 40.4 mmol, 63.6 eq) and DCM (3 mL) was stirred at 40° C. for 1 h. Then, the reaction mixture was concentrated under vacuum to give a residue. For purification 2 batches were combined (adding 170 mg) and the crude was purified by C₁₈ column chromatography (20 g, 0.1% NH₃·H₂O in water/MeCN, MeCN: 40%-50%) to give the title compound (370.9 mg, 79% yield) as yellow solid. LCMS m/z: 341.9 [M+H]⁺, ESI pos.

### Step B: 5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol

A mixture of 2-[(3R)-3-[(5-bromooxazolo[4,5-b]pyrazin-2-yl)amino]piperidino]ethanol;2,2,2-trifluoroacetic acid (324 mg, 710.2 μmol, 1.0 eq), 6-(methoxymethyl)-5-(4,4,5,5-tetramethyl-1,3,2-dioxaborolan-2-yl)-2,3-dihydrobenzofuran-4-ol Intermediate 8 (365 mg, 1.13 mmol, 1.6 eq), potassium carbonate (405 mg, 2.93 mmol, 4.126 eq) and 1,1′-bis(diphenylphosphino)ferrocene-palladium(ii)dichloride dichloromethane complex (89 mg, 109 μmol, 0.15 eq) in 1,4-dioxane (5.6 mL) and water (2.8 mL) was flushed with argon and stirred at 90° C. for 2 h. The reaction mixture was cooled to room temperature and extracted with ethyl acetate and half-saturated aq. NH₄Cl-solution. The aqueous layer was backextracted twice with ethyl acetate. The organic layers were washed with water and brine. The combined organic layers were dried over sodium sulfate, filtered and concentrated in vacuo. The crude product was purified by flash chromatography (silica gel, 25 g, gradient 0% to 10% methanol in dichloromethane) to afford the title compound (297 mg) as brown foam. After combining with another batch (35 mg) the compound was re-purified by flash chromatography (Si-amine, 12 g, gradient 0% to 10% methanol in ethyl acetate) followed by trituration with ethyl acetate/heptane to afford the title compound (206 mg, 63% yield) as light yellow powder. LCMS m/z: 442.4 [M+H]⁺, ESI pos.

### Example 12

### 5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol

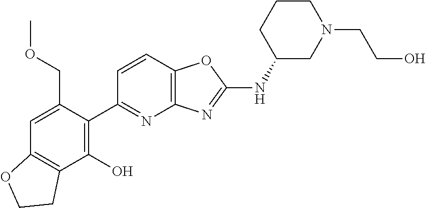 [SMILES: COCc1cc2c(c(O)c1-c1ccc3oc(N[C@@H]4CCCN(CCO)C4)nc3n1)CCO2]

A mixture of 2-[(3R)-3-[(5-chlorooxazolo[4,5-b]pyridin-2-yl)amino]piperidino]ethanol (124 mg, 397 μmol, 1.00 eq; CAS #2923540-56-9), 6-(methoxymethyl)-5-(4,4,5,5-tetramethyl-1,3,2-dioxaborolan-2-yl)-2,3-dihydrobenzofuran-4-ol Intermediate 8 (230 mg, 714 μmol, 1.80 eq), potassium carbonate (209 mg, 1.51 mmol, 3.81 eq) and 1,1′-bis(diphenylphosphino)ferrocene-palladium(ii) dichloride dichloromethane complex (50 mg, 61.23 μmol, 0.15 eq) in 1,4-dioxane (2.8 mL) and water (1.4 mL) was flushed with argon and stirred at 95° C. overnight. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was purified by flash chromatography (silica gel, 12 g, gradient 0% to 10% methanol in dichloromethane). All fractions containing product were combined and concentrated in vacuo and repurified by flash chromatography (Si-amine, 12 g, gradient 0% to 10% methanol in ethyl acetate) to afford the title compound (73 mg, 40% yield) as off-white foam. LCMS m/z: 441.3 [M+H]⁺, ESI pos.

### Example 13

### 5-[2-[[(3R)-1-(2-Hydroxyethyl)-3-piperidyl]amino]oxazolo[4,5-b]pyrazin-5-yl]-6-methyl-2,3-dihydrobenzofuran-4-ol

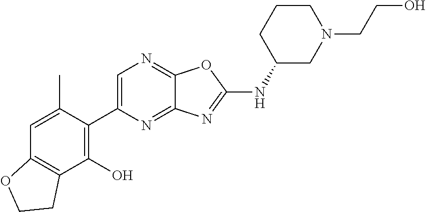 [SMILES: Cc1cc2c(c(O)c1-c1cnc3oc(N[C@@H]4CCCN(CCO)C4)nc3n1)CCO2]

### Step A: 5-[2-[[(3R)-1-[2-[tert-Butyl(dimethyl)silyl]oxyethyl]-3-piperidyl]amino]oxazolo[4,5-b]pyrazin-5-yl]-6-methyl-2,3-dihydrobenzofuran-4-ol

To a solution of 5-bromo-N-[(3R)-1-[2-[tert-butyl(dimethyl)silyl]oxyethyl]-3-piperidyl]oxazolo[4,5-b]pyrazin-2-amine (400.0 mg, 0.88 mmol, 1.0 eq, Example 10, Step D) in 1,4-dioxane (10 mL) and water (2 mL) was added 6-methyl-5-(4,4,5,5-tetramethyl-1,3,2-dioxaborolan-2-yl)-2,3-dihydrobenzofuran-4-ol (290.39 mg, 1.05 mmol, 1.2 eq; CAS #2923540-54-7) and CsF (332.79 mg, 2.19 mmol, 2.5 eq). Then XphosPdG₃ (74.27 mg, 0.09 mmol, 0.1 eq) was added to the above mixture. The reaction mixture was stirred at 80° C. for 4 h under N₂. Upon the reaction completion, the reaction mixture was cooled to room temperature. EtOAc (10 mL) and brine (3 mL) were added into the mixture and layers were separated. The aqueous phase was extracted with EtOAc (10 mL×3). The combined organic layers were dried over Na₂SO₄, filtered, and concentrated in vacuum. The residue was purified by silica gel column chromatography (petroleum ether:ethyl acetate=1:1 to 0:1) to afford the title compound (300 mg, 63% yield) as a yellow solid. LCMS m/z: 526.3 [M+H]⁺, ESI pos.

### Step B: 5-[2-[[(3R)-1-(2-Hydroxyethyl)-3-piperidyl]amino]oxazolo[4,5-b]pyrazin-5-yl]-6-methyl-2,3-dihydrobenzofuran-4-ol

A solution of 5-[2-[[(3R)-1-[2-[tert-butyl(dimethyl)silyl]oxyethyl]-3-piperidyl]amino]oxazolo[4,5-b]pyrazin-5-yl]-6-methyl-2,3-dihydrobenzofuran-4-ol (300.0 mg, 0.57 mmol, 1.0 eq) in DCM (3 mL) and TFA (3.0 mL, 40.39 mmol, 70.78 eq) was stirred at 40° C. for 2 hours. Upon the reaction completion, the reaction mixture was concentrated in vacuum and dissolved in MeOH (2 mL), basified to pH=7 by addition of saturated aqueous NaHCO₃ solution. Then the mixture was filtered, and the filtrate was concentrated under reduced pressure. The residue was purified by preparative HPLC (Method: Column Waters Xbridge 150×25 mm×5 μm; Condition: water (ammonia hydroxide v/v)-MeCN; Begin B: 0; End B: 30; Gradient Time (min): 10; 100% B Hold Time (min): 4; FlowRate (mL/min): 25) to afford the title compound (145.3 mg, 60% yield) as a white solid. LCMS m/z: 412.2 [M+H]⁺, ESI pos.

### Example 14

### 5-[2-[[(3R)-1-(2-Hydroxyethyl)-3-piperidyl]amino]oxazolo[4,5-b]pyridin-5-yl]-6-methyl-2,3-dihydrobenzofuran-4-ol

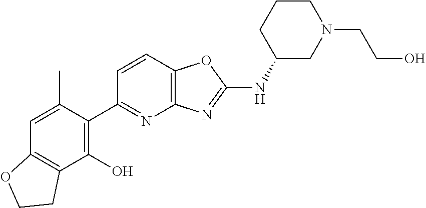 [SMILES: Cc1cc2c(c(O)c1-c1ccc3oc(N[C@@H]4CCCN(CCO)C4)nc3n1)CCO2]

A mixture of 2-[(3R)-3-[(5-chlorooxazolo[4,5-b]pyridin-2-yl)amino]piperidino]ethanol (70 mg, 224.1 μmol, 1.00 eq; CAS #2923540-56-9), 6-methyl-5-(4,4,5,5-tetramethyl-1,3,2-dioxaborolan-2-yl)coumaran-4-ol (129 mg, 350.4 μmol, 1.56 eq; CAS #2923540-54-7), potassium carbonate (146 mg, 1.06 mmol, 4.71 eq) and 1,1′-bis(diphenylphosphino)ferrocene-palladium(ii) dichloride dichloromethane complex (28 mg, 34.3 μmol, 0.15 eq) in 1,4-dioxane (1.5 mL) and water (750 μL) was flushed with argon and stirred at 95° C. overnight. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was purified by flash chromatography (silica gel, 12 g, gradient 0% to 10% methanol in dichloromethane). All fractions containing product were combined and concentrated in vacuo. The residue (52 mg) was submitted for further purification by SFC (Column: achiral 4-Ethyl Pyridine, 12 nm, 5 μm, 250×20 mm, Eluent B: 15% MeOH+0.2% DEA) to afford the title compound (16 mg, 17% yield) as light yellow solid. LCMS m/z: 411.1 [M+H]⁺, ESI pos.

### Example 15

### 3-Hydroxy-4-[2-[[(3R)-1-(2-hydroxyethyl)-3-piperidyl]amino]oxazolo[4,5-b]pyrazin-5-yl]-5-(methoxymethyl)benzonitrile;2,2,2-trifluoroacetic Acid

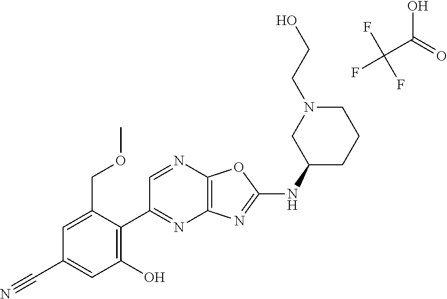

### Step A: 2-[(3R)-3-[(5-Bromooxazolo[4,5-b]pyrazin-2-yl)amino]-1-piperidyl]ethanol

To a solution of 1-(6-bromo-3-hydroxy-pyrazin-2-yl)-3-[(3R)-1-(2-hydroxyethyl)-3-piperidyl]thiourea Intermediate 11 (200.0 mg, 0.53 mmol, 1.0 eq), tetrabutylammonium iodide (39.3 mg, 0.11 mmol, 0.2 eq) in THF (5 mL) was added H₂02 (180.8 mg, 1.59 mmol, 3.0 eq, purity: 30% in water) and stirred at 20° C. for 1 h. Upon reaction completion, the mixture was quenched with saturated Na₂SO₃ aqueous solution (3 mL), then the mixture was concentrated in vacuum. The residue was purified by prep-HPLC (column: Xtimate C₁₈, 250 mm*50 mm*10 μm; mobile phase: [water (0.1% ammonia hydroxide v/v)-MeCN]; B %: 5%-35%, 10 minutes), the eluent was lyophilized to afford the title compound (80.0 mg, 39% yield) as a yellow solid. LCMS: m/z 344.0 [M+2+H]⁺, ESI pos.

### Step B: 4-[2-[[(3R)-1-(2-Hydroxyethyl)-3-piperidyl]amino]oxazolo[4,5-b]pyrazin-5-yl]-3-(methoxymethyl)-5-(2-trimethylsilylethoxymethoxy)benzonitrile

To a solution of aforementioned 2-[(3R)-3-[(5-bromooxazolo[4,5-b]pyrazin-2-yl)amino]-1-piperidyl]ethanol (50.0 mg, 0.15 mmol, 1.0 eq) and 3-(methoxymethyl)-4-(4,4,5,5-tetramethyl-1,3,2-dioxaborolan-2-yl)-5-(2-trimethylsilylethoxymethoxy)benzonitrile (91.9 mg, 0.22 mmol, 1.5 eq; CAS #2923541-74-4), CsF (66.6 mg, 0.44 mmol, 3.0 eq) in 1,4-dioxane (2 mL) and water (0.4 mL) was added Xphos Pd G₃ (24.8 mg, 0.03 mmol, 0.2 eq). Then the mixture was stirred at 80° C. for 2 h under nitrogen. Upon the reaction completion, the above reaction mixture was cooled to rt, and poured into ice (20 mL), and extracted with ethyl acetate (50 mL*3). The combined organic phases were washed with brine (50 mL*3), dried over anhydrous sodium sulfate, filtered and the filtrate was concentrated in vacuum. The residue was purified by silica gel column chromatography (petroleum ether:ethyl acetate=20:1 to 1:1) to afford the title compound (30.0 mg, 37% yield) as a yellow solid. ¹H NMR (400 MHz, CD₃OD) δ [ppm]: 7.78 (s, 1H), 7.56 (s, 1H), 7.54 (s, 1H), 5.22 (s, 2H), 4.31 (s, 2H), 4.07 (s, 1H), 3.71-3.67 (m, 2H), 3.60 (t, 2H), 3.25 (s, 3H), 3.00-2.96 (m, 1H), 2.61-2.59 (m, 1H), 2.59-2.56 (m, 2H), 2.47-2.45 (m, 2H), 1.89-1.83 (m, 2H), 1.70-1.66 (m, 2H), 0.86 (t, 2H), −0.05 (s, 9H).

### Step C: 3-Hydroxy-4-[2-[[(3R)-1-(2-hydroxyethyl)-3-piperidyl]amino]oxazolo[4,5-b]pyrazin-5-yl]-5-(methoxymethyl)benzonitrile;2,2,2-trifluoroacetic Acid

To a solution of aforementioned 4-[2-[[(3R)-1-(2-hydroxyethyl)-3-piperidyl]amino]oxazolo[4,5-b]pyrazin-5-yl]-3-(methoxymethyl)-5-(2-trimethylsilylethoxymethoxy)benzonitrile (15.0 mg, 0.03 mmol, 1.0 eq) in DCM (0.5 mL) as added TFA (0.5 mL). Then the mixture was stirred at 20° C. for 1 h. Upon the reaction completion, the above reaction mixture was concentrated in vacuum. The residue was purified by prep-HPLC (column: Phenomenex Gemini, 150 mm*30 mm*15 um; mobile phase: [water (0.1% TFA, V/V)-MeCN]; B %: 22%-52%, 12 mins), the eluent was lyophilized to afford the title compound (12.8 mg, 110% yield, TFA salt) as yellow oil. LCMS: m/z 425.3 [M+H]⁺, ESI pos.

### Example 16=Example 8 as Free Salt

### 3-Hydroxy-4-[2-[[(3R)-1-(2-hydroxyethyl)-3-piperidyl]amino]-7-methoxy-oxazolo[4,5-b]pyridin-5-yl]-5-methyl-benzonitrile

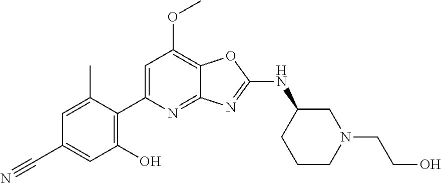 [SMILES: COc1cc(-c2c(C)cc(C#N)cc2O)nc2nc(N[C@@H]3CCCN(CCO)C3)oc12]

### Example 17=Examples 2 as Free Salt

### 4-[2-[[(3R,5S)-1-Ethyl-5-hydroxy-3-piperidyl]amino]oxazolo[4,5-b]pyrazin-5-yl]-3-hydroxy-5-methyl-benzonitrile

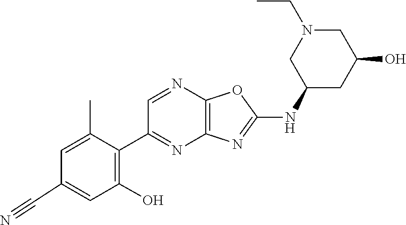

### Example 18

### 5-[2-[[(3R)-1-(2-Hydroxyethyl)-3-piperidyl]amino]oxazolo[4,5-b]pyrazin-5-yl]-6-methyl-indan-4-ol

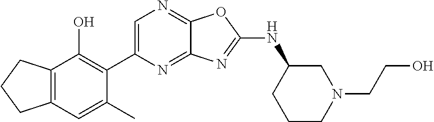 [SMILES: Cc1cc2c(c(O)c1-c1cnc3oc(N[C@@H]4CCCN(CCO)C4)nc3n1)CCC2]

### Step A: N-[(3R)-1-[2-[tert-butyl(dimethyl)silyl]oxyethyl]-3-piperidyl]-5-[6-methyl-4-(2-trimethylsilylethoxymethoxy)indan-5-yl]oxazolo[4,5-b]pyrazin-2-amine

To a solution of 5-bromo-N-[(3R)-1-[2-[tert-butyl(dimethyl)silyl]oxyethyl]-3-piperidyl]oxazolo[4,5-b]pyrazin-2-amine (110.0 mg, 0.11 mmol, 1.0 eq; Example 10, Step D), trimethyl-[2-[[6-methyl-5-(4,4,5,5-tetramethyl-1,3,2-dioxaborolan-2-yl)indan-4-yl]oxymethoxy]ethyl]silane (53.2 mg, 0.13 mmol, 1.2 eq; Example 19, Step G), CsF (66.6 mg, 0.44 mmol, 4.0 eq) in 1,4-dioxane (1 mL) and water (0.2 mL) was added Xphos Pd G₃ (18.6 mg, 0.02 mmol, 0.2 eq). The reaction vessel was sealed and heated in microwave at 95° C. for 3 h. Upon the reaction was completion, the reaction mixture was cooled to room temperature then diluted with water (20 mL) and extracted with ethyl acetate (30 mL*3). The combined organic phases were washed with brine (30 mL), dried over anhydrous sodium sulfate, filtered and the filtrate was concentrated under reduced pressure. The residue was purified by prep-TLC (ethyl acetate) to afford the title compound (50.0 mg, 42% yield) as yellow gum. LCMS: m/z 654.4 [M+H]⁺, ESI pos.

### Step B: 5-[2-[[(3R)-1-(2-Hydroxyethyl)-3-piperidyl]amino]oxazolo[4,5-b]pyrazin-5-yl]-6-methyl-indan-4-ol

A solution of N-[(3R)-1-[2-[tert-butyl(dimethyl)silyl]oxyethyl]-3-piperidyl]-5-[6-methyl-4-(2-trimethylsilylethoxymethoxy)indan-5-yl]oxazolo[4,5-b]pyrazin-2-amine (50.0 mg, 0.08 mmol, 1.0 eq) in HCl/dioxane (1.0 mL, 2M) was stirred at 25° C. for 1 h. Upon the reaction was completion, the above mixture was concentrated under reduced pressure, The residue was purified by prep-HPLC (column: Phenomenex Gemini, 150 mm*25 mm*10 um; mobile phase: [water (0.1% NH₄HCO₃, V/V)-MeCN]; B %: 22%-55%, 10 mins), then lyophilized to afford the title compound (5.1 mg, 16.0% yield) as a white solid. LCMS: m/z 410.3 [M+H]⁺, ESI pos.

### Example 19

### 5-[2-[[(3R)-1-(2-Hydroxyethyl)-3-piperidyl]amino]-7-methoxy-oxazolo[4,5-b]pyridin-5-yl]-6-methyl-indan-4-ol

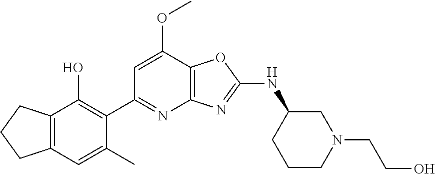 [SMILES: COc1cc(-c2c(C)cc3c(c2O)CCC3)nc2nc(N[C@@H]3CCCN(CCO)C3)oc12]

### Step A: 2-Vinylcyclopentene-1-carbaldehyde

To a solution of 2-bromocyclopentene-1-carbaldehyde (30.0 g, 171.4 mmol, 1.0 eq; CAS #89466-25-1), Cs₂CO₃ (167.6 g, 514.23 mmol, 3.0 eq), and potassium vinyltrifluoroborate (29.9 g, 222.8 mmol, 1.3 eq), PPh₃ (4.5 g, 17.1 mmol, 0.1 eq) in THF (500 mL) and water (100 mL) was added Pd(OAc)₂ (1.92 g, 8.57 mmol, 0.05 eq), the mixture was degassed with N₂ for 3 times and stirred at 50° C. for 12 h. The reaction mixture was diluted with water (500 mL), and extracted with EtOAc (300 mL×2). The combined organic phases were washed with brine (50 mL*3), dried over anhydrous sodium sulfate, filtered and the filtrate was concentrated in vacuum. The residue was purified by silica gel column chromatography (petroleum ether:ethyl acetate=1/0) to afford the title compound (15.0 g, 64% yield) as yellow oil. ¹H NMR (400 MHz, CDCl₃) δ [ppm]: 10.21 (s, 1H), 7.31-7.24 (m, 1H), 5.57 (d, 1H), 5.54 (s, 1H), 2.78 (t, 2H), 2.67 (t, 2H), 1.95-1.88 (m, 2H).

### Step B: 3-Methyl-1-(2-vinylcyclopenten-1-yl)but-3-en-1-ol

To a solution of aforementioned 2-vinylcyclopentene-1-carbaldehyde (6.0 g, 49.1 mmol, 1.0 eq) in THF (20 mL) was added 2-methylallylmagnesium chloride (197.0 mL, 98.5 mmol, 2.01 eq) at 0° C. under N₂, the mixture was stirred at 25° C. for 1 h. The reaction mixture was quenched with saturated NH₄Cl aqueous solution (150 mL), and extracted with EtOAc (100 mL×2). The combined organic layers were washed with brine (100 mL), dried over anhydrous sodium sulfate, filtered and the filtrate was concentrated in vacuum. The residue was purified by silica gel column chromatography (petroleum ether:ethyl acetate=1/0-10/1) to afford the title compound (2.5 g mg, 29% yield) as yellow oil. ¹H NMR (400 MHz, CDCl₃) δ [ppm]: 6.78-6.71 (m, 1H), 5.16-5.10 (m, 2H), 4.88 (m, 1H), 4.85-4.82 (m, 2H), 2.69-2.59 (m, 1H), 2.57-2.43 (m, 4H), 2.20 (dd, 1H), 1.90-1.82 (m, 2H), 1.79 (s, 3H).

### Step C: 3-Methyl-1-(2-vinylcyclopenten-1-yl)but-3-en-1-one

To a solution of aforementioned 3-methyl-1-(2-vinylcyclopenten-1-yl)but-3-en-1-ol (2.5 g mg, 14.02 mmol, 1.0 eq) in DCM (200 mL) was added Dess-Martin periodinane (11896.1 mg, 28.1 mmol, 2.0 eq) at 0° C. The mixture was stirred at 25° C. for 0.5 h. The reaction mixture was quenched with saturated Na₂SO₃ aqueous solution (150 mL), extracted with EtOAc (150 mL×2). The combined organic layers were washed with brine (100 mL), dried over anhydrous sodium sulfate, filtered and the filtrate was concentrated in vacuum. The residue was purified by silica gel column chromatography (petroleum ether:ethyl acetate=1/0 to 10/1) to afford the title compound (2.0 g, 65% yield) as yellow oil. ¹H NMR (400 MHz, CDCl₃) δ [ppm]: 7.41 (dd, 1H), 5.49-5.41 (m, 2H), 4.94 (s, 1H), 4.76 (d, 1H), 3.24 (s, 2H), 2.80 (t, 2H), 2.68 (t, 2H), 1.94-1.87 (m, 2H), 1.78 (s, 3H).

### Step D: 6-Methylindan-4-ol

To a solution of aforementioned 3-methyl-1-(2-vinylcyclopenten-1-yl)but-3-en-1-one (2000.0 mg, 11.4 mmol, 1.0 eq) in toluene (1000 mL) was added Hovey da-Grubbs (567.9 mg, 0.91 mmol, 0.08 eq), the mixture was degassed with N₂ for three times and the mixture was stirred at 80° C. for 12 h. The reaction mixture was concentrated under reduced pressure. The residue was purified by silica gel column chromatography (petroleum ether:ethyl acetate=20:1 to 5:1) to afford the title compound (1.2 g) as a yellow solid. ¹H NMR (400 MHz, CDCl₃): δ 6.67 (s, 1H), 6.46 (s, 1H), 4.54-4.51 (m, 1H), 2.89 (t, 2H), 2.82 (t, 2H), 2.29 (s, 3H), 2.14-2.06 (m, 2H).

### Step E: 5-Bromo-6-methyl-indan-4-ol

To a solution of aforementioned 6-methylindan-4-ol (1.2 g, 8.1 mmol, 1.0 eq) and i-Pr₂NH (163.6 mg, 1.62 mmol, 0.2 eq) in DCM (20 mL) was added NBS (1441.2 mg, 8.1 mmol, 1.0 eq) at 0° C. The mixture was stirred at 0° C. for 1 h. The reaction mixture was diluted with DCM (50 mL) and washed with brine (40 mL). The organic layers were separated and dried over Na₂SO₄, filtered and the filtrate was concentrated in vacuum. The residue was purified by silica gel column chromatography (petroleum ether:ethyl acetate=50:1 to 10:1) to afford the title compound (1.0 g, 54% yield) as a white solid. ¹H NMR (400 MHz, CDCl₃): δ 6.73 (s, 1H), 5.58 (s, 1H), 2.93-2.83 (m, 4H), 2.37 (s, 3H), 2.14-2.06 (m, 2H).

### Step F: 2-[(5-Bromo-6-methyl-indan-4-yl)oxymethoxy]ethyl-trimethyl-silane

To a solution of aforementioned 5-bromo-6-methyl-indan-4-ol (300.0 mg, 1.32 mmol, 1.0 eq) in DMF (5 mL) was added Cs₂CO₃ (860.8 mg, 2.64 mmol, 2.0 eq) at 0° C., then the mixture was stirred at 0° C. for 0.5 h. Then 2-(trimethylsilyl)ethoxymethyl chloride (0.26 mL, 1.45 mmol, 1.1 eq) was added to above mixture dropwise at 0° C., then the resulting mixture was stirred at 20° C. for 1 h. Upon reaction completion, the above reaction solution was diluted with water (10 mL), extracted with ethyl acetate (20 mL*3). The combined organic phases were washed with brine (20 mL), dried over anhydrous sodium sulfate, filtered and the filtrate was concentrated under reduced pressure. The residue was purified by silica gel column (petroleum ether:ethyl acetate=1:0 to 0:1) to afford the title compound (440.0 mg, 84% yield) as colorless oil. ¹H NMR (400 MHz, DMSO-d₆): δ 6.99 (s, 1H), 5.10 (s, 2H), 3.89-3.77 (m, 2H), 2.90 (t, 2H), 2.79 (t, 2H), 2.31 (s, 3H), 2.02-1.97 (m, 2H), 0.95-0.85 (m, 2H), 0.01 (s, 9H).

### Step G: Trimethyl-[2-[[6-methyl-5-(4,4,5,5-tetramethyl-1,3,2-dioxaborolan-2-yl)indan-4-yl]oxymethoxy]ethyl]silane

To a solution of aforementioned 2-[(5-bromo-6-methyl-indan-4-yl)oxymethoxy]ethyl-trimethyl-silane (440.0 mg, 1.23 mmol, 1.0 eq) and 4,4,4′,4′,5,5,5′,5′-octamethyl-2,2′-bi(1,3,2-dioxaborolane) (625.5 mg, 2.46 mmol, 2.0 eq), Cs₂CO₃ (802.4 mg, 2.46 mmol, 2.0 eq) and tris(4-methoxy-3,5-dimethylphenyl)phosphane (107.5 mg, 0.25 mmol, 0.2 eq) in 1,4-dioxane (10 mL) was added Pd(OAc)₂ (55.3 mg, 0.25 mmol, 0.2 eq) under N₂. The mixture was stirred at 95° C. for 3 h. Upon the reaction was completion, the above reaction mixture was cooled to rt, then diluted with water (30 mL) and ethyl acetate (20 mL), filtered and the filtrate was extracted with ethyl acetate (20 mL*3). The combined organic phases were washed with brine (20 mL), dried over anhydrous sodium sulfate, filtered and the filtrate was concentrated under reduced pressure. The residue was purified by prep-TLC (petroleum ether:ethyl acetate=20:1) to afford the title compound (330.0 mg, 66% yield) as colorless oil. ¹H NMR (400 MHz, CD₃OD) δ [ppm]: 6.81 (s, 1H), 5.07 (s, 2H), 4.60 (s, 2H), 3.75-3.88 (m, 2H), 2.75-3.03 (m, 4H), 2.30 (s, 3H), 1.92-2.11 (m, 2H), 1.37 (s, 12H), 1.34-1.43 (m, 1H), 0.82-1.00 (m, 2H), 0.00 (s, 9H).

### Step H: 7-methoxy-5-[6-methyl-4-(2-trimethylsilylethoxymethoxy)indan-5-yl]-N-[(3R)-1-[2-[tert-butyl(dimethyl)silyl]oxyethyl]-3-piperidyl]oxazolo[4,5-b]pyridin-2-amine

To a solution of N-[(3R)-1-[2-[tert-butyl(dimethyl)silyl]oxyethyl]-3-piperidyl]-5-chloro-7-methoxy-oxazolo[4,5-b]pyridin-2-amine (110.0 mg, 0.13 mmol, 1.0 eq, Example 8, Step F), in 1,4-dioxane (2 mL) and water (0.400 mL) was added aforementioned trimethyl-[2-[[6-methyl-5-(4,4,5,5-tetramethyl-1,3,2-dioxaborolan-2-yl)indan-4-yl]oxymethoxy]ethyl]silane (63.8 mg, 0.16 mmol, 1.2 eq), CsF (39.9 mg, 0.26 mmol, 2.0 eq) and Xphos Pd G3 (22.3 mg, 0.03 mmol, 0.2 eq). The reaction vessel was sealed and heated in microwave at 95° C. for 3 h. Upon reaction completion, the above reaction mixture was cooled to rt then diluted with water (20 mL), extracted with ethyl acetate (30 mL*3). The combined organic phases were washed with brine (30 mL), dried over anhydrous sodium sulfate, filtered and the filtrate was concentrated under reduced pressure. The residue was purified by prep-TLC (ethyl acetate) to afford the title compound (70.0 mg, 26% yield) as yellow gum. LCMS: m/z 683.6 [M+H]⁺, ESI pos.

### Step I: 5-[2-[[(3R)-1-(2-Hydroxyethyl)-3-piperidyl]amino]-7-methoxy-oxazolo[4,5-b]pyridin-5-yl]-6-methyl-indan-4-ol

A solution of 7-Methoxy-5-[6-methyl-4-(2-trimethylsilylethoxymethoxy)indan-5-yl]-N-[rac-(3R)-1-[2-[tert-butyl(dimethyl)silyl]oxyethyl]-3-piperidyl]oxazolo[4,5-b]pyridin-2-amine (70.0 mg, 0.1 mmol, 1.0 eq) in HCl/1,4-dioxane (1.0 mL, 2M) was stirred at 25° C. for 1 h. Upon the reaction was completion, the above mixture was concentrated under reduced pressure, then adjusted to pH=7 with saturated NaHCO₃ aqueous solution at 0° C., filtered and the filtrate was concentrated under reduced pressure. The residue was purified by prep-HPLC column: Phenomenex Gemini, 150 mm*25 mm*10 um; mobile phase: [water (0.1% NH₄HCO₃, V/V)-MeCN]; B %: 22%-52%, 12 mins), then lyophilized to afford the title compound (10.1 mg, 22% yield) as a white solid. LCMS: m/z 439.3 [M+H]⁺, ESI pos.

### Example 20

### 3-Hydroxy-4-[2-[[(3R)-1-(2-hydroxyethyl)-3-piperidyl]amino]-7-methoxy-oxazolo[4,5-b]pyridin-5-yl]-5-(methoxymethyl)benzonitrile

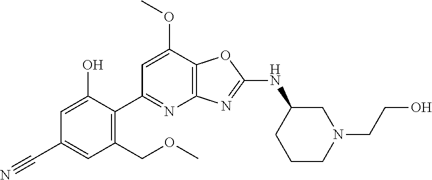 [SMILES: COCc1cc(C#N)cc(O)c1-c1cc(OC)c2oc(N[C@@H]3CCCN(CCO)C3)nc2n1]

### Step A: 3-(Methoxymethyl)-4-[7-methoxy-2-[[(3R)-1-[2-[tert-butyl(dimethyl)silyl]oxyethyl]-3-piperidyl]amino]oxazolo[4,5-b]pyridin-5-yl]-5-(2-trimethylsilylethoxymethoxy)benzonitrile

To a solution of N-[(3R)-1-[2-[tert-butyl(dimethyl)silyl]oxyethyl]-3-piperidyl]-5-chloro-7-methoxy-oxazolo[4,5-b]pyridin-2-amine (60.0 mg, 0.14 mmol, 1.0 eq; Example 8, Step F), 3-(methoxymethyl)-4-(4,4,5,5-tetramethyl-1,3,2-dioxaborolan-2-yl)-5-(2-trimethylsilylethoxy-methoxy)benzonitrile (68.5 mg, 0.16 mmol, 1.2 eq; CAS #2923541-74-4), CsF (41.3 mg, 0.27 mmol, 2.0 eq) in 1,4-dioxane (2 mL) and water (0.4 mL) was added Xphos Pd G₃ (23.1 mg, 0.03 mmol, 0.2 eq). The reaction vessel was sealed and heated in microwave at 95° C. for 1.5 h under nitrogen. Upon the reaction was completion, the above reaction mixture was cooled to room temperature and diluted with water (20 mL), extracted with ethyl acetate (30 mL*3). The combined organic phases were washed with brine (30 mL), dried over anhydrous sodium sulfate, filtered and the filtrate was concentrated under reduced pressure. The residue was purified by prep-TLC (ethyl acetate) to afford the title compound (50.0 mg, 35% yield) as yellow gum. ¹H NMR (400 MHz, CD₃OD) δ [ppm]: 7.55-7.52 (m, 1H), 7.51-7.48 (m, 1H), 6.72 (s, 1H), 5.20 (s, 2H), 4.86 (s, 1H), 4.25 (s, 2H), 4.06 (s, 3H), 3.98-3.92 (m, 1H), 3.82 (t, 2H), 3.59 (t, 2H), 3.34-3.32 (m, 1H), 3.27 (s, 3H), 3.25-3.18 (m, 1H), 2.86-2.74 (m, 1H), 2.66-2.55 (m, 2H), 2.35-2.23 (m, 2H), 2.06-1.99 (m, 1H), 1.86-1.77 (m, 1H), 1.74-1.65 (m, 1H), 1.55-1.42 (m, 1H), 0.87 (s, 9H), 0.07 (s, 6H), 0.00 (s, 9H).

### Step B: 3-Hydroxy-4-[2-[[(3R)-1-(2-hydroxyethyl)-3-piperidyl]amino]-7-methoxy-oxazolo[4,5-b]pyridin-5-yl]-5-(methoxymethyl)benzonitrile

A solution of aforementioned 3-(methoxymethyl)-4-[7-methoxy-2-[[(3R)-1-[2-[tert-butyl(dimethyl)silyl]oxyethyl]-3-piperidyl]amino]oxazolo[4,5-b]pyridin-5-yl]-5-(2-trimethylsilylethoxymethoxy)benzonitrile (50.0 mg, 0.07 mmol, 1.0 eq) in HCl/1,4-dioxane (1.0 mL, 2M) was stirred at 25° C. for 1 h. Upon reaction completion, the above mixture was adjusted to pH=7 with saturated NaHCO₃ aqueous solution at 0° C., filtered and the filtrate was concentrated under reduced pressure. The residue was purified by prep-HPLC (column: Phenomenex Gemini, 150 mm*25 mm*10 um; mobile phase: [water (0.1% NH₄HCO₃, V/V)-MeCN]; B %: 22%-52%, 11 mins), then lyophilized to afford the title compound (18.7 mg, 58% yield) as a white solid. LCMS: m/z 454.3 [M+H]⁺, ESI pos.

### Example 21

### 5-[2-[[(3R)-1-(2-Hydroxyethyl)-3-piperidyl]amino]-7-methoxy-oxazolo[4,5-b]pyridin-5-yl]-6-(methoxymethyl)-2,3-dihydrobenzofuran-4-ol

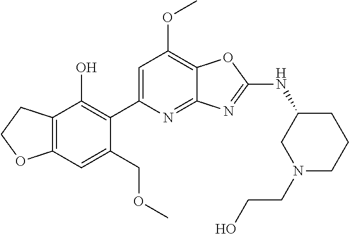 [SMILES: COCc1cc2c(c(O)c1-c1cc(OC)c3oc(N[C@@H]4CCCN(CCO)C4)nc3n1)CCO2]

### Step A: 6-(Methoxymethyl)-5-[7-methoxy-2-[[(3R)-1-[2-[tert-butyl(dimethyl)silyl]oxyethyl]-3-piperidyl]amino]oxazolo[4,5-b]pyridin-5-yl]-2,3-dihydrobenzofuran-4-ol

To a solution of 6-(methoxymethyl)-5-(4,4,5,5-tetramethyl-1,3,2-dioxaborolan-2-yl)-2,3-dihydrobenzofuran-4-ol Intermediate 8 (58.3 mg, 0.19 mmol, 1.2 eq) and N-[(3R)-1-[2-[tert-butyl(dimethyl)silyl]oxyethyl]-3-piperidyl]-5-chloro-7-methoxy-oxazolo[4,5-b]pyridin-2-amine (70.0 mg, 0.16 mmol, 1.0 eq; Example 8, Step F), K₂CO₃ (54.8 mg, 0.4 mmol, 2.5 eq) in 1,4-dioxane (1 mL) and water (0.200 mL) was added Xphos-PdG₃ (13.5 mg, 0.02 mmol, 0.1 eq). Then the reaction mixture was stirred at 95° C. for 4 h under N₂. Upon reaction completion, the reaction mixture was cooled to rt. Ethyl acetate (10 mL) and water (5 mL) were added, and the layers were separated. The aqueous phase was extracted with ethyl acetate (20 mL×3). Combined extracts were washed with brine (30 mL), dried over Na₂SO₄, filtered, and the filtrate was concentrated in vacuum. The residue was purified by silica gel column chromatography (petroleum ether:ethyl acetate=1:1 to 0:1) to afford the title compound (70.0 mg, 60% yield) as a yellow solid. LCMS: m/z 585.3 [M+H]⁺, ESI pos.

### Step B: 5-[2-[[(3R)-1-(2-Hydroxyethyl)-3-piperidyl]amino]-7-methoxy-oxazolo[4,5-b]pyridin-5-yl]-6-(methoxymethyl)-2,3-dihydrobenzofuran-4-ol

A solution of aforementioned 6-(methoxymethyl)-5-[7-methoxy-2-[[(3R)-1-[2-[tert-butyl(dimethyl)silyl]oxyethyl]-3-piperidyl]amino]oxazolo[4,5-b]pyridin-5-yl]-2,3-dihydro-benzofuran-4-ol (70.0 mg, 0.12 mmol, 1.0 eq) in HCl/dioxane (2M, 1.0 mL) was stirred at 25° C. for 30 min. Upon reaction completion, the reaction mixture was concentrated in vacuum. The residue was purified by prep-HPLC (column: Xtimate C₁₈, 150 mm*25 mm*5 μm; mobile phase: [water (0.1% ammonia hydroxide v/v)-MeCN]; B %: 10%-40%, 5 minutes) and lyophilized to afford the title compound (43.9 mg, 78% yield) as a white solid. LCMS: m/z 471.2 [M+H]⁺, ESI pos.

### Example 22

### 5-[2-[[(3R)-1-(2-Hydroxyethyl)-3-piperidyl]amino]-7-methoxy-oxazolo[4,5-b]pyridin-5-yl]-6-methyl-2,3-dihydrobenzofuran-4-ol

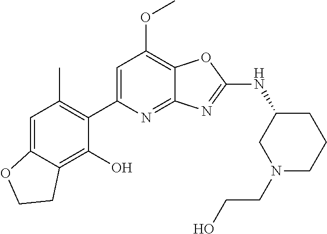 [SMILES: COc1cc(-c2c(C)cc3c(c2O)CCO3)nc2nc(N[C@@H]3CCCN(CCO)C3)oc12]

### Step A: 5-[7-Methoxy-2-[[(3R)-1-[2-[tert-butyl(dimethyl)silyl]oxyethyl]-3-piperidyl]amino]oxazolo[4,5-b]pyridin-5-yl]-6-methyl-2,3-dihydrobenzofuran-4-ol

To a solution of N-[(3R)-1-[2-[tert-butyl(dimethyl)silyl]oxyethyl]-3-piperidyl]-5-chloro-7-methoxy-oxazolo[4,5-b]pyridin-2-amine (50.0 mg, 0.11 mmol, 1.0 eq; Example 8, step F) and 6-methyl-5-(4,4,5,5-tetramethyl-1,3,2-dioxaborolan-2-yl)-2,3-dihydrobenzofuran-4-ol (37.6 mg, 0.14 mmol, 1.2 eq; CAS #2923540-54-7), K₂CO₃ (39.2 mg, 0.28 mmol, 2.5 eq) in 1,4-dioxane (1 mL) and water (0.2 mL) was added Xphos-PdG₃ (9.6 mg, 0.01 mmol, 0.1 eq). The reaction mixture was stirred at 90° C. for 2 h under N₂. Upon reaction completion, the reaction mixture was cooled to rt. Ethyl acetate (10 mL) and water (15 mL) were added to the reaction mixture and the layers were separated. The aqueous phase was extracted with ethyl acetate (10 mL×3). Combined extracts were washed with brine (5 mL×2), dried over Na₂SO₄, filtered, and the filtrate was concentrated in vacuum. The residue was purified by prep-TLC (EtOAc) to afford the title compound (50.0 mg, 77% yield) as a yellow solid. LCMS: m/z 555.3 [M+H]⁺, ESI pos.

### Step B: 5-[2-[[(3R)-1-(2-Hydroxyethyl)-3-piperidyl]amino]-7-methoxy-oxazolo[4,5-b]pyridin-5-yl]-6-methyl-2,3-dihydrobenzofuran-4-ol

A solution of aforementioned 5-[7-methoxy-2-[[(3R)-1-[2-[tert-butyl(dimethyl)silyl]oxyethyl]-3-piperidyl]amino]oxazolo[4,5-b]pyridin-5-yl]-6-methyl-2,3-dihydrobenzofuran-4-ol (45.0 mg, 0.08 mmol, 1.0 eq) in HCl/dioxane (2M, 1.0 mL) was stirred at 25° C. for 10 min. Upon reaction completion, the reaction mixture was concentrated in vacuum. The residue was dissolved in methanol (1 mL) and basified to pH=7 by saturated NaHCO₃ aqueous solution, then filtered, the filtrate was concentrated in vacuum. The residue was purified by prep-HPLC (column: Xtimate C₁₈, 150 mm*25 mm*5 um; mobile phase: [water (0.1% ammonia hydroxide v/v)-MeCN]; B %: 15%-45%, 10 minutes) and lyophilized to afford the title compound (16.9 mg, 46% yield) as a white solid. LCMS: m/z 441.2 [M+H]⁺, ESI pos.

### Example 23

### 4-[2-[[(3R,5S)-1-Ethyl-5-hydroxy-3-piperidyl]amino]oxazolo[4,5-b]pyrazin-5-yl]-3-hydroxy-5-(methoxymethyl)benzonitrile;2,2,2-trifluoroacetic Acid

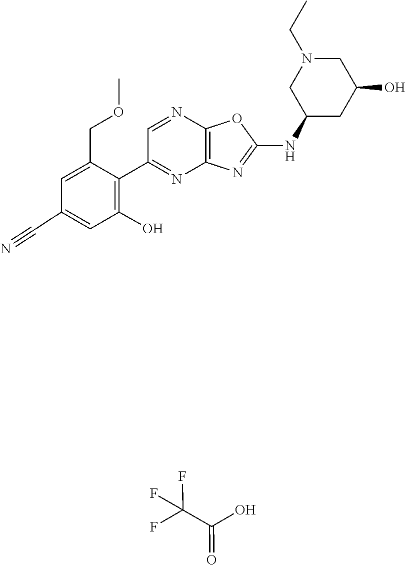

### Step A: 4-[2-[[(3R,5S)-1-ethyl-5-hydroxy-3-piperidyl]amino]oxazolo[4,5-b]pyrazin-5-yl]-3-(methoxymethyl)-5-(2-trimethylsilylethoxymethoxy)benzonitrile

To a solution of Intermediate 9 (3S,5R)-5-[(5-bromooxazolo[4,5-b]pyrazin-2-yl)amino]-1-ethyl-piperidin-3-ol (16.0 mg, 0.05 mmol, 1.0 eq), 3-(methoxymethyl)-4-(4,4,5,5-tetramethyl-1,3,2-dioxaborolan-2-yl)-5-(2-trimethylsilylethoxy methoxy)benzonitrile (21.57 mg, 0.05 mmol, 1.1 eq; CAS #2923541-74-4), K₂CO₃ (18.1 mg, 0.13 mmol, 2.8 eq) in 1,4-dioxane (1 mL) and water (0.2 mL) was added Xphos-PdG₃ (7.9 mg, 0.01 mmol, 0.2 eq). Then the reaction mixture was stirred at 95° C. for 2 h under N₂. Upon the reaction was completion, the above reaction mixture was cooled to rt, diluted with methanol (2 mL), filtered and the filtrate was concentrated under reduced pressure. The residue was purified by prep-TLC (methanol:ethyl acetate=1:5) to afford the title compound (16.0 mg, 55% yield) as a white solid. LCMS: m/z 555.3, [M+H]⁺, ESI pos.

### Step B: 4-[2-[[(3R,5S)-1-Ethyl-5-hydroxy-3-piperidyl]amino]oxazolo[4,5-b]pyrazin-5-yl]-3-hydroxy-5-(methoxymethyl)benzonitrile:2,2,2-trifluoroacetic Acid

To a solution of 4-[2-[[(3R,5S)-1-ethyl-5-hydroxy-3-piperidyl]amino]oxazolo[4,5-b]pyrazin-5-yl]-3-(methoxymethyl)-5-(2-trimethylsilylethoxymethoxy)benzonitrile (14.0 mg, 0.03 mmol, 1.0 eq) in DCM (1 mL) was added trifluoroacetic acid (1.0 mL), then the mixture was stirred at 20° C. for 1 h. Upon reaction completion, the above reaction solution was concentrated under reduced pressure. The residue was purified by prep-HPLC (column: Phenomenex Gemini, 150 mm*30 mm*15 um; mobile phase: [water (0.1% TFA, V/V)-MeCN]; B %: 22%-50%, 11 mins), and lyophilized to afford the title compound (10.45 mg, 77% yield) as yellow gum. LCMS: m/z 425.3, [M+H]⁺, ESI pos.

### Example 24

### (3S,5R)-1-(2-Hydroxyethyl)-5-[[5-(4-hydroxy-6-methyl-2,3-dihydrobenzofuran-5-yl)oxazolo[4,5-b]pyrazin-2-yl]amino]piperidin-3-ol

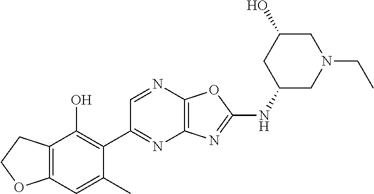 [SMILES: CCN1C[C@@H](O)C[C@@H](Nc2nc3nc(-c4c(C)cc5c(c4O)CCO5)cnc3o2)C1]

### Step A: (3S,5R)-5-[(5-bromooxazolo[4,5-b]pyrazin-2-yl)amino]-1-[2-[tert-butyl(dimethyl)silyl]oxyethyl]piperidin-3-ol

To a solution of (3S,5R)-5-[(5-bromooxazolo[4,5-b]pyrazin-2-yl)amino]piperidin-3-ol (120.0 mg, 0.22 mmol, 1.0 eq; Intermediate 9, Step D), CH₃COOH (33.7 mg, 0.56 mmol, 2.5 eq), CH₃COONa (55.2 mg, 0.67 mmol, 3.0 eq) in DCM (2 mL) and methanol (0.200 mL) was added (tert-butyldimethylsilyloxy)acetaldehyde (58.6 mg, 0.34 mmol, 1.5 eq). The reaction mixture was stirred at 20° C. for 5 min. Then NaBH(OAc)₃ (237.6 mg, 1.12 mmol, 5.0 eq) was added to the above reaction mixture and stirred at 20° C. for 30 min. Upon the reaction completion, the reaction mixture was quenched with water (10 mL) and extracted with ethyl acetate (10 mL×4). Combined extracts were washed with brine (10 mL×2), dried over Na₂SO₄, filtered, and the filtrate was concentrated in vacuum. The residue was purified by silica gel column chromatography (ethyl acetate:methanol=1:0 to 10:1) to afford the title compound (30.0 mg, 35% yield) as a white solid. ¹H NMR (400 MHz, CD₃OD) δ [ppm]: 7.90 (s, 1H), 4.04-3.96 (m, 1H), 3.81 (t, 2H), 3.26-3.20 (m, 1H), 3.09-3.01 (m, 1H), 2.64 (t, 2H), 2.36-2.29 (m, 1H), 2.18-2.04 (m, 2H), 1.46-1.27 (m, 2H), 0.88 (s, 9H), 0.08 (s, 6H)).

### Step B: (3S,5R)-1-[2-[tert-butyl(dimethyl)silyl]oxyethyl]-5-[[5-(4-hydroxy-6-methyl-2,3-dihydrobenzofuran-5-yl)oxazolo[4,5-b]pyrazin-2-yl]amino]piperidin-3-ol

To a solution of aforementioned (3S,5R)-5-[(5-bromooxazolo[4,5-b]pyrazin-2-yl)amino]-1-[2-[tert-butyl(dimethyl)silyl]oxyethyl]piperidin-3-ol (20.0 mg, 0.04 mmol, 1.0 eq) and 6-methyl-5-(4,4,5,5-tetramethyl-1,3,2-dioxaborolan-2-yl)-2,3-dihydrobenzofuran-4-ol (14.0 mg, 0.05 mmol, 1.2 eq; CAS #2923540-54-7), K₂CO₃ (14.6 mg, 0.11 mmol, 2.5 eq) in 1,4-dioxane (1 mL) and water (0.2 mL) was added XphosPdG₃ (7.2 mg, 0.01 mmol, 0.2 eq). The reaction mixture was stirred at 90° C. for 2 h under N₂. Upon the reaction completion, the reaction mixture was cooled to room temperature. Ethyl acetate (10 mL) and water (5 mL) were added to the mixture and the layers were separated. The aqueous phase was extracted with ethyl acetate (10 mL×4). Combined extracts were washed with brine (30 mL), dried over Na₂SO₄, filtered, and the filtrate was concentrated in vacuum. The residue was purified by prep-TLC (EtOAc) to afford the title compound (20.0 mg, 1.0 eq) as yellow oil. LCMS: m/z 542.2 [M+H]⁺, ESI pos.

### Step C: (3S,5R)-1-(2-Hydroxyethyl)-5-[[5-(4-hydroxy-6-methyl-2,3-dihydrobenzofuran-5-yl)oxazolo[4,5-b]pyrazin-2-yl]amino]piperidin-3-ol

A solution of (3S,5R)-1-[2-[tert-butyl(dimethyl)silyl]oxyethyl]-5-[[5-(4-hydroxy-6-methyl-2,3-dihydrobenzofuran-5-yl)oxazolo[4,5-b]pyrazin-2-yl]amino]piperidin-3-ol (20.0 mg, 0.04 mmol, 1.0 eq) and TFA (0.5 mL) in DCM (0.5 mL) was stirred at 40° C. for 2 h. Upon reaction completion, the reaction mixture was concentrated in vacuum. The residue was dissolved in methanol (1 mL) and basified to pH=7 by saturated NaHCO₃ aqueous solution, then the mixture was purified by prep-HPLC (column: Xtimate C₁₈, 250 mm*50 mm*10 μm; mobile phase: [water (0.1% ammonia hydroxide v/v)-MeCN]; B %: 5%-45%, 12 minutes) and lyophilized to afford the title compound (12.2 mg, 77% yield) as a yellow solid. LCMS: m/z 428.2 [M+H]⁺, ESI pos.

### Example 25

### (3S,5R)-1-Ethyl-5-[[5-(4-hydroxy-6-methyl-2,3-dihydrobenzofuran-5-yl)oxazolo[4,5-b]pyrazin-2-yl]amino]piperidin-3-ol

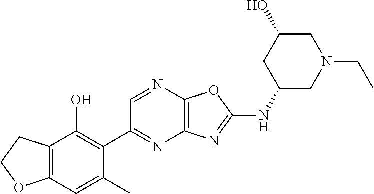 [SMILES: CCN1C[C@@H](O)C[C@@H](Nc2nc3nc(-c4c(C)cc5c(c4O)CCO5)cnc3o2)C1]

To a solution of Intermediate 9 (3S,5R)-5-[(5-bromooxazolo[4,5-b]pyrazin-2-yl)amino]-1-ethyl-piperidin-3-ol (20.0 mg, 0.06 mmol, 1.0 eq), 6-methyl-5-(4,4,5,5-tetramethyl-1,3,2-dioxaborolan-2-yl)-2,3-dihydrobenzofuran-4-ol (19.4 mg, 0.07 mmol, 1.2 eq; CAS #2923540-54-7), K₂CO₃ (22.7 mg, 0.16 mmol, 2.8 eq) in 1,4-dioxane (1 mL) and water (0.2 mL) was added Xphos-PdG₃ (9.9 mg, 0.01 mmol, 0.2 eq), then the mixture was stirred at 90° C. for 1 h under N₂. Upon reaction completion, the above reaction mixture was cooled to rt, diluted with methanol (2 mL), filtered and the filtrate was concentrated under reduced pressure. The residue was purified by prep-HPLC (column: Phenomenex Gemini, 150 mm*25 mm*10 um; mobile phase: [water (0.1% NH₄HCO₃, V/V)-MeCN]; B %: 22%-52%, 12 mins), then the eluent was lyophilized to afford the title compound (7.28 mg, 30% yield) as a white solid. LCMS: m/z 412.2 [M+H]⁺, ESI pos.

### Example 26=Example 15 as Free Salt

### 3-Hydroxy-4-[2-[[(3R)-1-(2-hydroxyethyl)-3-piperidyl]amino]oxazolo[4,5-b]pyrazin-5-yl]-5-(methoxymethyl)benzonitrile

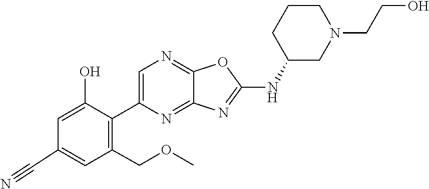

### Example 27

### 4-[2-[[(3R,5S)-1-Ethyl-5-hydroxy-3-piperidyl]amino]-7-fluoro-oxazolo[4,5-b]pyridin-5-yl]-3-hydroxy-5-methyl-benzonitrile

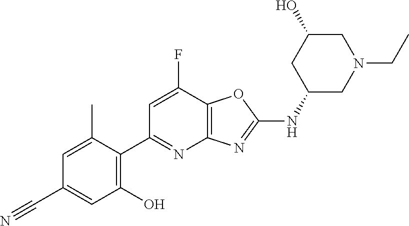 [SMILES: CCN1C[C@@H](O)C[C@@H](Nc2nc3nc(-c4c(C)cc(C#N)cc4O)cc(F)c3o2)C1]

### Step A: tert-Butyl (3R,5S)-3-[(5-chloro-7-fluoro-oxazolo[4,5-b]pyridin-2-yl)amino]-5-hydroxy-piperidine-1-carboxylate

To a solution of (3R,5S)-3-amino-5-hydroxy-piperidine-1-carboxylic acid tert-butyl ester (275.8 mg, 1.28 mmol, 1.2 eq), 2,5-dichloro-7-fluoro-oxazolo[4,5-b]pyridine (220.0 mg, 1.06 mmol, 1.0 eq) in MeCN (4 mL) was added DIPEA (274.2 mg, 2.13 mmol, 2.0 eq), then the mixture was stirred at 20° C. for 0.5 hour. Upon the reaction was completion, the above reaction solution was concentrated under reduced pressure. The residue was purified by column chromatography (SiO₂, petroleum ether:ethyl acetate=1:0 to 0:1) to afford the title compound (380.0 mg, 97% yield) as a yellow solid. LCMS: m/z 387.1, [M+H]⁺, ESI pos.

### Step B: tert-butyl (3R,5S)-3-[[5-[4-Cyano-2-methyl-6-(2-trimethylsilylethoxymethoxy)phenyl]-7-fluoro-oxazolo[4,5-b]pyridin-2-yl]amino]-5-hydroxy-piperidine-1-carboxylate

To a solution of tert-butyl (3R,5S)-3-[(5-chloro-7-fluoro-oxazolo[4,5-b]pyridin-2-yl)amino]-5-hydroxy-piperidine-1-carboxylate (370.0 mg, 0.96 mmol, 1.0 eq), Intermediate 1 3-methyl-4-(4,4,5,5-tetramethyl-1,3,2-dioxaborolan-2-yl)-5-(2-trimethylsilylethoxymethoxy)benzonitrile (558.7 mg, 1.43 mmol, 1.5 eq), K₂CO₃ (371.0 mg, 2.68 mmol, 2.8 eq) in 1,4-dioxane (8 mL) and water (1 mL) was added XphosPdG₃, (121.6 mg, 0.14 mmol, 0.15 eq), then the mixture was stirred at 95° C. for 2 hours under N₂. Upon the reaction was completion, the above reaction solution was cooled to room temperature, diluted with water (100 mL), and extracted with ethyl acetate (50 mL×3). The combined organic phases were washed with brine (100 mL), dried over anhydrous sodium sulfate, filtered and the filtrate was concentrated under reduced pressure. The residue was purified by column chromatography (SiO₂, petroleum ether:ethyl acetate=1:0 to 0:1) to afford the title compound (500.0 mg, 82% yield) as a yellow solid. LCMS: m/z 614.3, [M+H]⁺, ESI pos.

### Step C: 4-[7-Fluoro-2-[[(3R,5S)-5-hydroxy-3-piperidyl]amino]oxazolo[4,5-b]pyridin-5-yl]-3-hydroxy-5-methyl-benzonitrile

To a solution of tert-butyl (3R,5S)-3-[[5-[4-cyano-2-methyl-6-(2-trimethylsilylethoxymethoxy)phenyl]-7-fluoro-oxazolo[4,5-b]pyridin-2-yl]amino]-5-hydroxy-piperidine-1-carboxylate (100.0 mg, 0.16 mmol, 1.0 eq) in DCM (1 mL) was added TFA (1.0 mL), then the mixture was stirred at 20° C. for 1 hour. Upon the reaction was completion, the above reaction solution was concentrated under reduced pressure to afford the title compound (TFA salt, 81.0 mg, 0.16 mmol, 99.95% yield) as yellow gum. LCMS: m/z 384.1, [M+H]⁺, ESI pos.

### Step D: 4-[2-[[(3R,5S)-1-Ethyl-5-hydroxy-3-piperidyl]amino]-7-fluoro-oxazolo[4,5-b]pyridin-5-yl]-3-hydroxy-5-methyl-benzonitrile

To a solution of 4-[7-fluoro-2-[[(3R,5S)-5-hydroxy-3-piperidyl]amino]oxazolo[4,5-b]pyridin-5-yl]-3-hydroxy-5-methyl-benzonitrile (TFA salt, 81.0 mg, 0.16 mmol, 1.0 eq), DIPEA (84.1 mg, 0.65 mmol, 4.0 eq) in DMF (1 mL) was added iodoethane (24.5 mg, 0.16 mmol, 1.0 eq), then the mixture was stirred at 20° C. for 12 hours. Upon the reaction mixture was completion, the reaction mixture was concentrated in vacuum. The residue was purified by prep-HPLC (column: Xtimate C18, 250 mm*50 mm*10 μm; mobile phase: [water (0.1% ammonia hydroxide v/v)-MeCN]; B %: 5%-35%, 10 minutes) and followed by lyophilization to afford the title compound (10.02 mg, 0.02 mmol, 14.66% yield) as a white solid. LCMS: m/z 412.1, [M+H]⁺, ESI pos.

### Example 28

### 3-Hydroxy-5-methyl-4-[2-[[(3R)-1-(3-oxazol-2-ylpropyl)-3-piperidyl]amino]oxazolo[4,5-b]pyridin-5-yl]benzonitrile

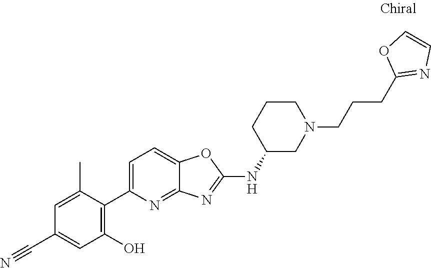 [SMILES: Cc1cc(C#N)cc(O)c1-c1ccc2oc(N[C@@H]3CCCN(CCCc4ncco4)C3)nc2n1]

### Step A: Methyl (E)-3-oxazol-2-ylprop-2-enoate

To an ice-cold solution of methyl diethylphosphonoacetate (CAS #1067-74-9, 1.12 g, 0.97 mL, 5.15 mmol, 1.05 eq) in tetrahydrofuran (15.2 mL) was added sodium hydride 60% in mineral oil (206 mg, 5.15 mmol, 1.05 eq) and the mixture was stirred at this temperature for 10 minutes. A solution of oxazole-2-carbaldehyde (CAS #65373-52-6, 500 mg, 4.89 mmol, 1.00 eq) in tetrahydrofuran (34.2 mL) was added dropwise. Stirring was continued at 0° C. for 1.5 hours. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 24 g, gradient 0% to 50% ethyl acetate in heptane) to afford the title compound (591 mg, 75% yield) as a white solid. LCMS: m/z 153.9 [M+H]⁺, ESI pos.

### Step B: Methyl 3-oxazol-2-ylpropanoate

A solution of methyl (E)-3-oxazol-2-ylprop-2-enoate (Example 28, step A) (591 mg, 3.67 mmol, 1.00 eq) in methanol (18 mL) and ethyl acetate (9 mL) was three times alternating evacuated and flushed with argon. Palladium on activated charcoal (195 mg, 0.18 mmol, 0.05 eq, 10% purity) was added carefully. The reaction flask was evacuated, flushed with argon, evacuated and flushed with hydrogen. The reaction mixture was stirred under hydrogen atmosphere (balloon) at room temperature for 3 hours. The reaction mixture was filtered over celite and rinsed well with ethyl acetate/methanol. The filtrate was concentrated to afford the title compound (346 mg, 58% yield) as a colorless oil, which was used without further purification. LCMS: m/z 155.9 [M+H]⁺, ESI pos.

### Step C: 3-Oxazol-2-ylpropan-1-ol

A solution of methyl 3-oxazol-2-ylpropanoate (Example 28, step B) (150 mg, 0.92 mmol, 1.00 eq) in tetrahydrofuran (5.8 mL) was cooled to 0° C. before lithium aluminum hydride (1.0 M solution in tetrahydrofuran) (1.0 mL, 1.00 mmol, 1.09 eq) was added dropwise at 0° C. (gas evolution observed). The reaction mixture was stirred at 0° C. for 2 hours. Sodium sulfate decahydrate was added carefully to quench the reaction. When no more bubbling was detected, the ice bath was removed, sodium sulfate was added and the mixture was stirred for 1 hour at room temperature. The suspension was filtered over celite and rinsed with ethyl acetate/methanol. The filtrate was concentrated in vacuo. The crude product was adsorbed on ISOLUTE HM-N and purified by flash chromatography (silica gel, 4 g, gradient 0% to 100% ethyl acetate in heptane) to afford the title compound (45 mg, 35% yield, 90% purity) as a colorless oil. LCMS: m/z 128.1 [M+H]⁺, ESI pos.

### Step D: 3-Oxazol-2-ylpropyl 4-methylbenzenesulfonate

To a solution of 3-oxazol-2-ylpropan-1-ol (Example 28, step C) (45 mg, 0.32 mmol, 1.00 eq, 90% purity) in dichloromethane (1.0 mL) was added triethylamine (81 mg, 0.11 mL, 0.80 mmol, 2.51 eq) followed by p-toluenesulfonyl chloride (73 mg, 0.38 mmol, 1.20 eq). The reaction mixture was stirred at room temperature for 6 hours. The reaction mixture was poured into saturated aq. NaHCO₃-solution and extracted three times with dichlormethane. The combined organic layers were washed with brine, dried over sodium sulfate, filtered and concentrated in vacuo to afford the title compound (108 mg, 84% yield, 70% purity) as a light red oil, which was used without further purification. LCMS: m/z 282.2 [M+H]⁺, ESI pos.

### Step E: 5-Chloro-N-[(3R)-1-(3-oxazol-2-ylpropyl)-3-piperidyl]oxazolo[4,5-b]pyridin-2-amine

To a mixture of 5-chloro-N-[(3R)-3-piperidyl]oxazolo[4,5-b]pyridin-2-amine (Example 4, step B) (55 mg, 0.21 mmol, 1.00 eq) and 3-oxazol-2-ylpropyl 4-methylbenzenesulfonate (Example 28, step D) (108 mg, 0.27 mmol, 1.30 eq, 70% purity) in tetrahydrofuran (1.0 mL) and N,N-dimethylformamide (1.0 mL) was added N,N-diisoproylethylamine (80 mg, 0.11 mL, 0.62 mmol, 2.99 eq). The reaction mixture was stirred at 50° C. for 16 hours and at 70° C. for 6 hours. The reaction mixture was cooled to room temperature and then extracted with ethyl acetate and 10% aq. LiCl-solution. The aqueous layer was backextracted with ethyl acetate. The organic layers were washed two times with 10% aq. LiCl-solution, once with water and once with brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in ethyl acetate) to afford the title compound (34 mg, 39% yield, 85% purity) as a colorless oil. LCMS: m/z 362.2 [M+H]⁺, ESI pos.

### Step F: 3-Hydroxy-5-methyl-4-[2-[[(3R)-1-(3-oxazol-2-ylpropyl)-3-piperidyl]amino]oxazolo[4,5-b]pyridin-5-yl]benzonitrile

A mixture of 5-chloro-N-[(3R)-1-(3-oxazol-2-ylpropyl)-3-piperidyl]oxazolo[4,5-b]pyridin-2-amine (Example 28, step E) (34 mg, 0.09 mmol, 1.00 eq), (4-cyano-2-hydroxy-6-methyl-phenyl)boronic acid, Intermediate 7, (26 mg, 0.15 mmol, 1.56 eq), cesium carbonate (98 mg, 0.30 mmol, 3.19 eq) and XPhos Pd G₃ (15 mg, 0.02 mmol, 0.19 eq) in 1,4-dioxane (0.94 mL) and water (0.24 mL) was flushed with argon and stirred at 100° C. for 5 hours. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichlormethane) to afford the title compound (25 mg, 55% yield) as a light yellow solid. LCMS: m/z 459.3 [M+H]⁺, ESI pos.

### Example 29

### 4-[7-Fluoro-2-[[(3R)-1-(2-hydroxyethyl)-3-piperidyl]amino]oxazolo[4,5-b]pyridin-5-yl]-3-hydroxy-5-methyl-benzonitrile

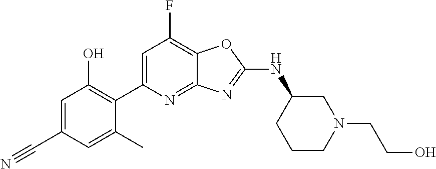 [SMILES: Cc1cc(C#N)cc(O)c1-c1cc(F)c2oc(N[C@@H]3CCCN(CCO)C3)nc2n1]

### Step A: N-[(3R)-1-[2-[tert-Butyl(dimethyl)silyl]oxyethyl]-3-piperidyl]-5-chloro-7-fluoro-oxazolo[4,5-b]pyridin-2-amine

To a solution of ((3R)-1-[2-[tert-butyl(dimethyl)silyl]oxyethyl]piperidin-3-amine (164.8 mg, 0.64 mmol, 1.2 eq), DIPEA (137.1 mg, 1.06 mmol, 2.0 eq) in MeCN (2 mL) was added Intermediate 10 2,5-dichloro-7-fluoro-oxazolo[4,5-b]pyridine (110.0 mg, 0.53 mmol, 1.0 eq), then the mixture was stirred at 20° C. for 2 hours. Upon the reaction was completion, the above reaction solution was diluted with water (50 mL), and extracted with ethyl acetate (20 mL×3). The combined organic phases were washed with brine (50 mL), dried over anhydrous sodium sulfate, filtered and the filtrate was concentrated under reduced pressure. The residue was purified by column chromatography (SiO₂, petroleum ether:ethyl acetate=1:1 to 0:1) to afford the title compound (200.0 mg, 82% yield) as a yellow solid. LCMS: m/z 429.1, [M+H]⁺, ESI pos.

### Step B: 4-[2-[[(3R)-1-[2-[tert-Butyl(dimethyl)silyl]oxyethyl]-3-piperidyl]amino]-7-fluoro-oxazolo[4,5-b]pyridin-5-yl]-3-methyl-5-(2-trimethylsilylethoxymethoxy)benzonitrile

To a solution of N-[(3R)-1-[2-[tert-butyl(dimethyl)silyl]oxyethyl]-3-piperidyl]-5-chloro-7-fluoro-oxazolo[4,5-b]pyridin-2-amine (200.0 mg, 0.47 mmol, 1.0 eq), Intermediate 1 3-methyl-4-(4,4,5,5-tetramethyl-1,3,2-dioxaborolan-2-yl)-5-(2-trimethylsilylethoxymethoxy)benzonitrile (272.3 mg, 0.7 mmol, 1.5 eq), K₂CO₃ (180.8 mg, 1.31 mmol, 2.8 eq) in 1,4-dioxane (4 mL) and water (0.5 mL) was added and XphosPdG₃ (59.3 mg, 0.07 mmol, 0.15 eq). Then the mixture was stirred at 95° C. for 2 hours under N₂. Upon the reaction was completion, the above reaction solution was cooled to room temperature, diluted with water (100 mL), and extracted with ethyl acetate (50 mL×3). The combined organic phases were washed with brine (100 mL), dried over anhydrous sodium sulfate, filtered and the filtrate was concentrated under reduced pressure, The residue was purified by column chromatography (SiO₂, petroleum ether:ethyl acetate=1:1 to 0:1) to afford the title compound (300.0 mg, 93% yield) as a yellow solid. LCMS: m/z 656.3, [M+H]⁺, ESI pos.

### Step C: 4-[7-Fluoro-2-[[(3R)-1-(2-hydroxyethyl)-3-piperidyl]amino]oxazolo[4,5-b]pyridin-5-yl]-3-hydroxy-5-methyl-benzonitrile

A solution of 4-[2-[[(3R)-1-[2-[tert-butyl(dimethyl)silyl]oxyethyl]-3-piperidyl]amino]-7-fluoro-oxazolo[4,5-b]pyridin-5-yl]-3-methyl-5-(2-trimethylsilylethoxymethoxy)benzonitrile (100.0 mg, 0.15 mmol, 1.0 eq) in trifluoroacetic acid (2.0 mL) was stirred at 45° C. for 3 hours. Upon the reaction was completion, the above reaction solution was concentrated under reduced pressure and then the residue dissolved with DMF (1 mL) and adjusted the pH=8 with NH₃·H₂O (25% in water), then purified by reversed-phase flash (CombiFlash [water (0.1% ammonia hydroxide v/v)-MeCN]) to afford 4-[7-fluoro-2-[[(3R)-1-(2-hydroxyethyl)-3-piperidyl]amino]oxazolo[4,5-b]pyridin-5-yl]-3-hydroxy-5-methyl-benzoni trile (34.39 mg, 0.08 mmol, 54.17% yield) as a white solid. LCMS: m/z 412.1, [M+H]⁺, ESI pos.

### Example 30

### 4-[2-[[(3R,5S)-1-Ethyl-5-hydroxy-3-piperidyl]amino]-7-methoxy-oxazolo[4,5-b]pyridin-5-yl]-3-hydroxy-5-methyl-benzonitrile

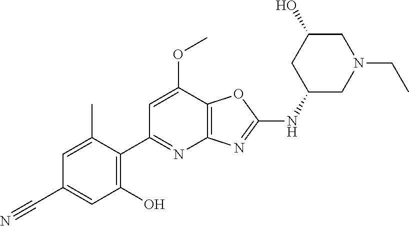 [SMILES: CCN1C[C@@H](O)C[C@@H](Nc2nc3nc(-c4c(C)cc(C#N)cc4O)cc(OC)c3o2)C1]

### Step A: tert-butyl (3R,5S)-3-[(5-Chloro-7-methoxy-oxazolo[4,5-b]pyridin-2-yl)amino]-5-hydroxy-piperidine-1-carboxylate

To a solution of tert-butyl (3R,5S)-3-amino-5-hydroxypiperidine-1-carboxylate (54.3 mg, 0.25 mmol, 1.1 eq CAS:1932513-59-1) and 2,5-dichloro-7-methoxy-oxazolo[4,5-b]pyridine (50.0 mg, 0.23 mmol, 1.0 eq, Example 8, Step E) in MeCN (0.5 mL) was added DIEA (58.9 mg, 0.46 mmol, 2.0 eq). Then the mixture was stirred at 20° C. for 0.5 hour. Upon the reaction completion, the mixture was concentrated in vacuum. The residue was purified by silica gel column chromatography (petroleum ether:ethyl acetate=1:3 to 1:5) to afford the title compound (75.0 mg, 82% yield) as a white solid. LCMS: m/z 399.2 [M+H]⁺, ESI pos.

### Step B: (3S,5R)-5-[(5-Chloro-7-methoxy-oxazolo[4,5-b]pyridin-2-yl)amino]piperidin-3-ol

To a solution of tert-butyl (3R,5S)-3-[(5-chloro-7-methoxy-oxazolo[4,5-b]pyridin-2-yl)amino]-5-hydroxy-piperidine-1-carboxylate (75.0 mg, 0.19 mmol, 1.0 eq) in DCM (0.5 mL) was added TFA (0.5 mL). The mixture was stirred at 20° C. for 2 hours. Upon the reaction completion, the reaction mixture was concentrated in vacuum. The residue was purified by reversed phase flash (CombiFlash, mobile phase: [water (0.1% TFA v/v)-MeCN]; B %: 30%-40%) and following by lyophilization to afford the title compound (TFA salt, 67.0 mg, 86% yield) as colorless oil. LCMS: m/z 299.1 [M+H]⁺, ESI pos.

### Step C: (3S,5R)-5-[(5-Chloro-7-methoxy-oxazolo[4,5-b]pyridin-2-yl)amino]-1-ethyl-piperidin-3-ol

To a solution of (3S,5R)-5-[(5-chloro-7-methoxy-oxazolo[4,5-b]pyridin-2-yl)amino]piperidin-3-ol (TFA salt, 67.0 mg, 0.16 mmol, 1.0 eq) and TEA (16.4 mg, 0.16 mmol, 1.0 eq), AcOH (14.6 mg, 0.24 mmol, 1.5 eq) in DCM (1 mL)/methanol (0.2 mL) was added acetaldehyde (0.16 mL, 0.81 mmol, 5.0 eq). The mixture was stirred at 20° C. for 0.5 hour. Then NaBH(OAc)₃ (103.2 mg, 0.49 mmol, 3.0 eq) was added to the above reaction mixture. Then the mixture was stirred at 20° C. for 1 hour under nitrogen. Upon the reaction completion, the mixture was concentrated in vacuum. The residue was purified reversed phase flash (CombiFlash, mobile phase: [water (0.10% TFA v/v)-MeCN]; B %: 35%-50%) and following by lyophilization to afford the title compound (TFA salt, 35.0 mg, 41% yield) as yellow oil. LCMS: m/z 327.1 [M+H]⁺, ESI, pos.

### Step D: 4-[2-[[(3R,5S)-1-Ethyl-5-hydroxy-3-piperidyl]amino]-7-methoxy-oxazolo[4,5-b]pyridin-5-yl]-3-methyl-5-(2-trimethylsilylethoxymethoxy)benzonitrile

To a solution of (3S,5R)-5-[(5-chloro-7-methoxy-oxazolo[4,5-b]pyridin-2-yl)amino]-1-ethyl-piperidin-3-ol (TFA salt, 35.0 mg, 0.07 mmol, 1.0 eq) and Intermediate 1 3-methyl-4-(4,4,5,5-tetramethyl-1,3,2-dioxaborolan-2-yl)-5-(2-trimethylsilylethoxymethoxy)benzonitrile (39.6 mg, 0.1 mmol, 1.5 eq) in 1,4-dioxane (1 mL)/water (0.1 mL) was added K₂CO₃ (26.3 mg, 0.19 mmol, 2.8 eq) and Xphos-PdG₃ (5.8 mg, 0.01 mmol, 0.1 eq) under N2. The reaction mixture was stirred at 95° C. for 12 hours. Upon the reaction completion, the mixture was cooled to 20° C. and concentrated in vacuum. The residue was purified by silica gel column chromatography (petroleum ether:ethyl acetate=1:2 to 0:1) to afford the title compound (25.0 mg, 63% yield) as yellow oil. LCMS: m/z 554.3 [M+H]⁺, ESI pos.

### Step E: 4-[2-[[(3R,5S)-1-Ethyl-5-hydroxy-3-piperidyl]amino]-7-methoxy-oxazolo[4,5-b]pyridin-5-yl]-3-hydroxy-5-methyl-benzonitrile

A mixture of 4-[2-[[(3R,5S)-1-ethyl-5-hydroxy-3-piperidyl]amino]-7-methoxy-oxazolo[4,5-b]pyridin-5-yl]-3-methyl-5-(2-trimethylsilylethoxymethoxy)benzonitrile (23.0 mg, 0.04 mmol, 1.0 eq), TFA (0.1 mL) in DCM (1.0 mL) was stirred at 40° C. for 2 hours. Upon the reaction completion, the mixture was cooled to 20° C. and concentrated in vacuum. The residue was dissolved with MeOH (2 mL) and adjusted to pH=8 with NH₃·H₂O (25% in water). Then the mixture was purified by prep-HPLC (Method: Column: Waters Xbridge 150*25 mm*5 μm, mobile phase: [water (0.1% ammonia hydroxide v/v)-MeCN]; B %: 14%-44%, 10 minutes). The eluent was lyophilized to afford the title compound (6.1 mg, 34% yield) as a white solid. LCMS: m/z 424.1 [M+H]⁺, ESI pos.

### Example 31

### (3S,5R)-1-Ethyl-5-[[5-(4-hydroxy-6-methyl-2,3-dihydrobenzofuran-5-yl)oxazolo[4,5-b]pyridin-2-yl]amino]piperidin-3-ol

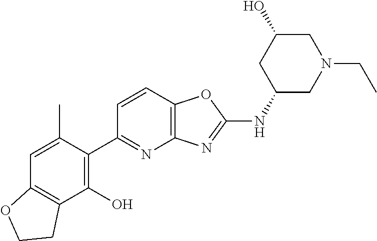 [SMILES: CCN1C[C@@H](O)C[C@@H](Nc2nc3nc(-c4c(C)cc5c(c4O)CCO5)ccc3o2)C1]

A mixture of (3S,5R)-5-[(5-chlorooxazolo[4,5-b]pyridin-2-yl)amino]-1-ethyl-piperidin-3-ol (CAS #2923539-98-2, 65 mg, 0.20 mmol, 1.00 eq, 90% purity), 6-methyl-5-(4,4,5,5-tetramethyl-1,3,2-dioxaborolan-2-yl)-2,3-dihydrobenzofuran-4-ol (CAS #2923540-54-7, 117 mg, 0.32 mmol, 1.61 eq, 75% purity), potassium carbonate (120 mg, 0.87 mmol, 4.40 eq) and 1,1′-bis(diphenylphosphino)ferrocene-palladium(ii) dichloride dichloromethane complex (25 mg, 0.03 mmol, 0.16 eq) in 1,4-dioxane (1.4 mL) and water (0.70 mL) was flushed with argon and stirred at 95° C. for 16 hours. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane). All fractions containing product were combined and concentrated in vacuo. The residue was submitted for further purification by SFC (column achiral DEA; eluent B: 15% methanol) to afford the title compound (24 mg, 28% yield) as a light yellow solid. LCMS: m/z 411.3 [M+H]⁺, ESI pos.

### Example A

A compound of formula I can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula I can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

1. A compound of formula I 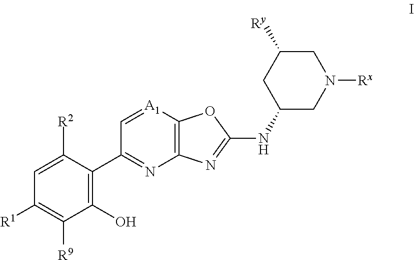
wherein,
R¹ is cyano, halo, or haloalkyl, and R⁹ is H, or
R¹ and R⁹ and the atoms to which they are attached form a 5-membered cycloalkyl or a 5-membered heterocycle comprising one O heteroatom;
R² is alkyl, hydroxyalkyl, alkoxyalkyl or cyano, wherein if R¹ is cyano then R² is not cyano;
A₁ is —CR¹⁰— or —N—;
R¹⁰ is H, fluoro, or alkoxy;
R^{x} is H, alkyl, heteroarylalkyl, hydroxyalkyl, or COOH-alkyl, wherein the heteroaryl of the heteroarylalkyl is oxazolyl, tetrazolyl, or oxadiazolyl;
R^{y} is H or —OH;
wherein, if R¹ is haloalkyl, then R^{x} is hydroxyalkyl or COOH-alkyl and R^{y} is —OH;
and pharmaceutically acceptable salts thereof, provided that the compound is not a compound selected from:
5-Chloro-2-[2-[[(3R)-1-ethyl-3-piperidyl]amino]oxazolo[4,5-b]pyridin-5-yl]-3-methyl-phenol;
5-Chloro-2-[2-[[(3R)-1-ethyl-3-piperidyl]amino]oxazolo[4,5-b]pyridin-5-yl]-3-hydroxy-benzonitrile;
5-Chloro-2-[2-[[(3R)-1-ethyl-3-piperidyl]amino]oxazolo[4,5-b]pyridin-5-yl]-3-(methoxymethyl)phenol;2,2,2-trifluoroacetic acid;
5-Chloro-2-[2-[[(3R)-1-ethyl-3-piperidyl]amino]oxazolo[4,5-b]pyridin-5-yl]-3-(methoxymethyl)phenol;
5-Chloro-3-methyl-2-[2-[[(3R)-1-methyl-3-piperidyl]amino]oxazolo[4,5-b]pyridin-5-yl]phenol;
5-Chloro-3-methyl-2-[2-[[(3R)-1-propyl-3-piperidyl]amino]oxazolo[4,5-b]pyridin-5-yl]phenol;
(3S,5R)-5-[[5-(4-Chloro-2-hydroxy-6-methyl-phenyl)oxazolo[4,5-b]pyridin-2-yl]amino]-1-methyl-piperidin-3-ol;
(3R,5R)-5-[[5-(4-Chloro-2-hydroxy-6-methyl-phenyl)oxazolo[4,5-b]pyridin-2-yl]amino]-1-ethyl-piperidin-3-ol;formic acid;
(3R,5R)-5-[[5-(4-Chloro-2-hydroxy-6-methyl-phenyl)oxazolo[4,5-b]pyridin-2-yl]amino]-1-ethyl-piperidin-3-ol;
5-Chloro-2-[2-[[(3R)-1-(3,3-difluoropropyl)-3-piperidyl]amino]oxazolo[4,5-b]pyridin-5-yl]-3-methyl-phenol;
5-Chloro-2-[2-[[(3R)-1-(2-hydroxyethyl)-3-piperidyl]amino]oxazolo[4,5-b]pyridin-5-yl]-3-methyl-phenol;
5-Chloro-3-(hydroxymethyl)-2-[2-[[(3R)-1-methyl-3-piperidyl]amino]oxazolo[4,5-b]pyridin-5-yl]phenol;
5-Chloro-2-[2-[[(3R)-1-(3-hydroxypropyl)-3-piperidyl]amino]oxazolo[4,5-b]pyridin-5-yl]-3-methyl-phenol;
5-Chloro-3-methyl-2-[2-[[(3R)-1-(2-hydroxypropyl)-3-piperidyl]amino]oxazolo[4,5-b]pyridin-5-yl]phenol;
(3S,5R)-5-[[5-[4-Chloro-2-hydroxy-6-(methoxymethyl)phenyl]oxazolo[4,5-b]pyridin-2-yl]amino]-1-methyl-piperidin-3-ol;
5-Chloro-2-[2-[[(3R)-1-ethyl-3-piperidyl]amino]oxazolo[4,5-b]pyrazin-5-yl]-3-methyl-phenol;2,2,2-trifluoroacetic acid;
5-Chloro-2-[2-[[(3R)-1-ethyl-3-piperidyl]amino]oxazolo[4,5-b]pyrazin-5-yl]-3-methyl-phenol;
(3S,5R)-5-[[5-(4-Chloro-2-hydroxy-6-methyl-phenyl)oxazolo[4,5-b]pyridin-2-yl]amino]-1-ethyl-piperidin-3-ol;
2-[2-[[(3R)-1-Ethyl-3-piperidyl]amino]oxazolo[4,5-b]pyridin-5-yl]-5-fluoro-3-methyl-phenol;
5-Fluoro-3-methyl-2-[2-[[(3R)-1-methyl-3-piperidyl]amino]oxazolo[4,5-b]pyridin-5-yl]phenol;
4-[2-[[(3R)-1-Ethyl-3-piperidyl]amino]oxazolo[4,5-b]pyridin-5-yl]-3-hydroxy-5-methyl-benzonitrile;
3-Hydroxy-5-methyl-4-[2-[[(3R)-1-methyl-3-piperidyl]amino]oxazolo[4,5-b]pyridin-5-yl]benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;formic acid;
4-[2-[[(3R,5S)-1-Ethyl-5-hydroxy-3-piperidyl]amino]oxazolo[4,5-b]pyridin-5-yl]-3-hydroxy-5-methyl-benzonitrile;
3-Hydroxy-4-[2-[[(3R,5S)-5-hydroxy-1-methyl-3-piperidyl]amino]oxazolo[4,5-b]pyridin-5-yl]-5-methyl-benzonitrile;
4-[2-[[(3R,5R)-1-Ethyl-5-hydroxy-3-piperidyl]amino]oxazolo[4,5-b]pyridin-5-yl]-3-hydroxy-5-methyl-benzonitrile;
3-Hydroxy-4-[2-[[(3R)-1-(2-hydroxyethyl)-3-piperidyl]amino]oxazolo[4,5-b]pyridin-5-yl]-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid 4-[2-[[(3R)-1-Ethyl-3-piperidyl]amino]oxazolo[4,5-b]pyridin-5-yl]-3-hydroxy-5-(methoxymethyl)benzonitrile;
4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid;
4-[2-[[(3R)-1-Ethyl-3-piperidyl]amino]-7-methoxy-oxazolo[4,5-b]pyridin-5-yl]-3-hydroxy-5-methyl-benzonitrile;
5-[2-[[(3R)-1-Ethyl-3-piperidyl]amino]oxazolo[4,5-b]pyridin-5-yl]-6-methyl-2,3-dihydrobenzofuran-4-ol;
5-Chloro-3-methyl-2-[2-[[(3R)-3-piperidyl]amino]oxazolo[4,5-b]pyridin-5-yl]phenol;dihydrochloride;
5-Chloro-3-methyl-2-[2-[[(3R)-3-piperidyl]amino]oxazolo[4,5-b]pyridin-5-yl]phenol;
and pharmaceutically acceptable salts thereof.

2-3. (canceled)

4. A compound according to claim 1, wherein R¹ and R⁹ and the atoms to which they are attached form a 5-membered heterocycle comprising one O heteroatom.

5. A compound according to claim 1, wherein R² is alkyl or alkoxyalkyl.

6. A compound according to claim 1, wherein R¹⁰ is H or alkoxy.

7-13. (canceled)

14. A compound according to claim 1, wherein,
R¹ is cyano or haloalkyl, and R⁹ is H, or
R¹ and R⁹ and the atoms to which they are attached form a 5-membered heterocycle comprising one O heteroatom;
R² is alkyl or alkoxyalkyl;
A₁ is —CR¹⁰— or —N—;
R¹⁰ is H or alkoxy;
R^{x} is H, alkyl, heteroarylalkyl, hydroxyalkyl, or COOH-alkyl, wherein the heteroaryl of the heteroarylalkyl is oxazolyl;
R^{y} is H or —OH;
wherein, if R¹ is haloalkyl, then R^{x} is hydroxyalkyl or COOH-alkyl and R^{y} is —OH, and pharmaceutically acceptable salts thereof.

15. A compound according to claim 1, wherein,
R¹ is cyano and R⁹ is H, or
R¹ and R⁹ and the atoms to which they are attached form a 5-membered heterocycle comprising one O heteroatom;
R² is alkyl or alkoxyalkyl;
A₁ is —CR¹⁰— or —N—;
R¹⁰ is H or alkoxy;
R^{x} is alkyl or hydroxyalkyl;
R^{y} is H or —OH;
and pharmaceutically acceptable salts thereof.

16-17. (canceled)

18. A compound according to claim 1, selected from:
3-Hydroxy-5-methyl-4-[2-[[(3R)-1-(2-oxazol-2-ylethyl)-3-piperidyl]amino]oxazolo[4,5-b]pyridin-5-yl]benzonitrile;
3-Hydroxy-4-[2-[[(3R)-1-(2-hydroxyethyl)-3-piperidyl]amino]oxazolo[4,5-b]pyridin-5-yl]-5-(methoxymethyl)benzonitrile;
4-[2-[[(3R,5S)-1-Ethyl-5-hydroxy-3-piperidyl]amino]oxazolo[4,5-b]pyrazin-5-yl]-3-hydroxy-5-methyl-benzonitrile;2,2,2-trifluoroacetic acid;
3-Hydroxy-4-[2-[[(3R)-1-(2-hydroxyethyl)-3-piperidyl]amino]oxazolo[4,5-b]pyrazin-5-yl]-5-methyl-benzonitrile;2,2,2-trifluoroacetic acid;
and pharmaceutically acceptable salts thereof.

19. A compound according to claim 1, selected from:
4-[2-[[(3R,5S)-1-Ethyl-5-hydroxy-3-piperidyl]amino]oxazolo[4,5-b]pyrazin-5-yl]-3-hydroxy-5-methyl-benzonitrile;
3-Hydroxy-4-[2-[[(3R)-1-(2-hydroxyethyl)-3-piperidyl]amino]oxazolo[4,5-b]pyrazin-5-yl]-5-methyl-benzonitrile;
and pharmaceutically acceptable salts thereof.

20. A compound according to claim 1, selected from:
4-[(3S,5R)-3-Hydroxy-5-[[5-[2-hydroxy-6-methyl-4-(trifluoromethyl)phenyl]oxazolo[4,5-b]pyridin-2-yl]amino]-1-piperidyl]butanoic acid;
(3S,5R)-1-(2-Hydroxyethyl)-5-[[5-[2-hydroxy-6-methyl-4-(trifluoromethyl)phenyl]oxazolo[4,5-b]pyridin-2-yl]amino]piperidin-3-ol;
3-Hydroxy-4-[2-[[(3R,5S)-5-hydroxy-1-methyl-3-piperidyl]amino]oxazolo[4,5-b]pyrazin-5-yl]-5-methyl-benzonitrile;2,2,2-trifluoroacetic acid;
3-Hydroxy-4-[2-[[(3R)-1-(2-hydroxyethyl)-3-piperidyl]amino]-7-methoxy-oxazolo[4,5-b]pyridin-5-yl]-5-methyl-benzonitrile;2,2,2-trifluoroacetic acid;
3-Hydroxy-4-[2-[[(3R,5S)-5-hydroxy-3-piperidyl]amino]oxazolo[4,5-b]pyridin-5-yl]-5-methyl-benzonitrile;
3-Hydroxy-4-[2-[[(3R)-1-(2-hydroxyethyl)-3-piperidyl]amino]oxazolo[4,5-b]pyrazin-5-yl]-5-methyl-benzonitrile;
and pharmaceutically acceptable salts thereof.

21. A compound according to claim 1, selected from:
(3S,5R)-1-(2-Hydroxyethyl)-5-[[5-[2-hydroxy-6-methyl-4-(trifluoromethyl)phenyl]oxazolo[4,5-b]pyridin-2-yl]amino]piperidin-3-ol;
3-Hydroxy-4-[2-[[(3R,5S)-5-hydroxy-1-methyl-3-piperidyl]amino]oxazolo[4,5-b]pyrazin-5-yl]-5-methyl-benzonitrile;
3-Hydroxy-4-[2-[[(3R)-1-(2-hydroxyethyl)-3-piperidyl]amino]-7-methoxy-oxazolo[4,5-b]pyridin-5-yl]-5-methyl-benzonitrile;
and pharmaceutically acceptable salts thereof.

22. A compound according to claim 1, selected from:
5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyrazin-5-yl]-6-methyl-2,3-dihydrobenzofuran-4-ol;
5-[2-[[(3R)-1-(2-Hydroxyethyl)-3-piperidyl]amino]oxazolo[4,5-b]pyridin-5-yl]-6-methyl-2,3-dihydrobenzofuran-4-ol;
and pharmaceutically acceptable salts thereof.

23. A compound according to claim 1, selected from:
3-hydroxy-4-[2-[[(3R)-1-(2-hydroxyethyl)-3-piperidyl]amino]oxazolo[4,5-b]pyrazin-5-yl]-5-(methoxymethyl)benzonitrile;2,2,2-trifluoroacetic acid;
3-hydroxy-4-[2-[[(3R)-1-(2-hydroxyethyl)-3-piperidyl]amino]oxazolo[4,5-b]pyrazin-5-yl]-5-(methoxymethyl)benzonitrile;
3-hydroxy-4-[2-[[(3R)-1-(2-hydroxyethyl)-3-piperidyl]amino]-7-methoxy-oxazolo[4,5-b]pyridin-5-yl]-5-methyl-benzonitrile;
4-[2-[[(3R,5S)-1-ethyl-5-hydroxy-3-piperidyl]amino]oxazolo[4,5-b]pyrazin-5-yl]-3-hydroxy-5-methyl-benzonitrile;
5-[2-[[(3R)-1-(2-hydroxyethyl)-3-piperidyl]amino]oxazolo[4,5-b]pyrazin-5-yl]-6-methyl-indan-4-ol;
5-[2-[[(3R)-1-(2-hydroxyethyl)-3-piperidyl]amino]-7-methoxy-oxazolo[4,5-b]pyridin-5-yl]-6-methyl-indan-4-ol;
3-hydroxy-4-[2-[[(3R)-1-(2-hydroxyethyl)-3-piperidyl]amino]-7-methoxy-oxazolo[4,5-b]pyridin-5-yl]-5-(methoxymethyl)benzonitrile;
5-[2-[[(3R)-1-(2-hydroxyethyl)-3-piperidyl]amino]-7-methoxy-oxazolo[4,5-b]pyridin-5-yl]-6-(methoxymethyl)-2,3-dihydrobenzofuran-4-ol;
5-[2-[[(3R)-1-(2-hydroxyethyl)-3-piperidyl]amino]-7-methoxy-oxazolo[4,5-b]pyridin-5-yl]-6-methyl-2,3-dihydrobenzofuran-4-ol;
4-[2-[[(3R,5S)-1-ethyl-5-hydroxy-3-piperidyl]amino]oxazolo[4,5-b]pyrazin-5-yl]-3-hydroxy-5-(methoxymethyl)benzonitrile;2,2,2-trifluoroacetic acid;
4-[2-[[(3R,5S)-1-ethyl-5-hydroxy-3-piperidyl]amino]oxazolo[4,5-b]pyrazin-5-yl]-3-hydroxy-5-(methoxymethyl)benzonitrile;
(3S,5R)-1-(2-hydroxyethyl)-5-[[5-(4-hydroxy-6-methyl-2,3-dihydrobenzofuran-5-yl)oxazolo[4,5-b]pyrazin-2-yl]amino]piperidin-3-ol;
(3S,5R)-1-ethyl-5-[[5-(4-hydroxy-6-methyl-2,3-dihydrobenzofuran-5-yl)oxazolo[4,5-b]pyrazin-2-yl]amino]piperidin-3-ol;
3-hydroxy-4-[2-[[(3R)-1-(2-hydroxyethyl)-3-piperidyl]amino]oxazolo[4,5-b]pyrazin-5-yl]-5-(methoxymethyl)benzonitrile;
4-[2-[[(3R,5S)-1-ethyl-5-hydroxy-3-piperidyl]amino]-7-fluoro-oxazolo[4,5-b]pyridin-5-yl]-3-hydroxy-5-methyl-benzonitrile;
3-hydroxy-5-methyl-4-[2-[[(3R)-1-(3-oxazol-2-ylpropyl)-3-piperidyl]amino]oxazolo[4,5-b]pyridin-5-yl]benzonitrile;
4-[7-fluoro-2-[[(3R)-1-(2-hydroxyethyl)-3-piperidyl]amino]oxazolo[4,5-b]pyridin-5-yl]-3-hydroxy-5-methyl-benzonitrile;
4-[2-[[(3R,5S)-1-ethyl-5-hydroxy-3-piperidyl]amino]-7-methoxy-oxazolo[4,5-b]pyridin-5-yl]-3-hydroxy-5-methyl-benzonitrile;
(3S,5R)-1-ethyl-5-[[5-(4-hydroxy-6-methyl-2,3-dihydrobenzofuran-5-yl)oxazolo[4,5-b]pyridin-2-yl]amino]piperidin-3-ol;
and pharmaceutically acceptable salts thereof.

24. A compound according to claim 1, selected from:
4-[2-[[(3R,5S)-1-Ethyl-5-hydroxy-3-piperidyl]amino]oxazolo[4,5-b]pyrazin-5-yl]-3-hydroxy-5-methyl-benzonitrile;2,2,2-trifluoroacetic acid;
4-[2-[[(3R,5S)-1-Ethyl-5-hydroxy-3-piperidyl]amino]oxazolo[4,5-b]pyrazin-5-yl]-3-hydroxy-5-methyl-benzonitrile;
3-Hydroxy-4-[2-[[(3R)-1-(2-hydroxyethyl)-3-piperidyl]amino]oxazolo[4,5-b]pyrazin-5-yl]-5-methyl-benzonitrile;2,2,2-trifluoroacetic acid;
3-Hydroxy-4-[2-[[(3R)-1-(2-hydroxyethyl)-3-piperidyl]amino]oxazolo[4,5-b]pyrazin-5-yl]-5-methyl-benzonitrile;
5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyrazin-5-yl]-6-methyl-2,3-dihydrobenzofuran-4-ol;
5-[2-[[(3R)-1-(2-Hydroxyethyl)-3-piperidyl]amino]oxazolo[4,5-b]pyridin-5-yl]-6-methyl-2,3-dihydrobenzofuran-4-ol;
and pharmaceutically acceptable salts thereof.

25. A compound according to claim 1, selected from:
3-Hydroxy-4-[2-[[(3R)-1-(2-hydroxyethyl)-3-piperidyl]amino]oxazolo[4,5-b]pyrazin-5-yl]-5-methyl-benzonitrile;2,2,2-trifluoroacetic acid;
3-Hydroxy-4-[2-[[(3R)-1-(2-hydroxyethyl)-3-piperidyl]amino]oxazolo[4,5-b]pyrazin-5-yl]-5-methyl-benzonitrile;
5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyrazin-5-yl]-6-methyl-2,3-dihydrobenzofuran-4-ol;
5-[2-[[(3R)-1-(2-Hydroxyethyl)-3-piperidyl]amino]oxazolo[4,5-b]pyridin-5-yl]-6-methyl-2,3-dihydrobenzofuran-4-ol;
and pharmaceutically acceptable salts thereof.

26. A compound according to claim 1, selected from:
5-[2-[[(3R)-1-(2-Hydroxyethyl)-3-piperidyl]amino]oxazolo[4,5-b]pyrazin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyridin-5-yl]-6-(methoxymethyl)-2,3-dihydrobenzofuran-4-ol;
5-[2-[[(3R)-1-(2-Hydroxyethyl)-3-piperidyl]amino]oxazolo[4,5-b]pyrazin-5-yl]-6-methyl-2,3-dihydrobenzofuran-4-ol;
5-[2-[[(3R)-1-(2-Hydroxyethyl)-3-piperidyl]amino]oxazolo[4,5-b]pyridin-5-yl]-6-methyl-2,3-dihydrobenzofuran-4-ol;
and pharmaceutically acceptable salts thereof.

27. (canceled)

28. A pharmaceutical composition comprising a compound according to claim 1 and a therapeutically inert carrier.

29-38. (canceled)

39. A method of inhibiting NLRP3 in a patient in need thereof which method comprises administering to the patient an effective amount of a compound according to claim 1, thereby inhibiting NLRP3.

40. A method for the treatment or prophylaxis of a disease, disorder or condition in a patient in need thereof which method comprises administering to the patient an effective amount of a compound according to claim 1, wherein the disease, disorder or condition is selected from asthma and chronic obstructive pulmonary disease.

41. A method for the treatment or prophylaxis of a cardiovascular disease, disorder or condition in a patient in need thereof which method comprises administering to the patient an effective amount of a compound according to claim 1.

42. A method for the treatment or prophylaxis of a cardiometabolic disease, disorder or condition in a patient in need thereof which method comprises administering to the patient an effective amount of a compound according to claim 1.
